(19) United States
(12) Patent Application Publication
(10) Pub. No.: US 2026/0132187 A1
(43) Pub. Date: May 14, 2026

(54) SARS-CoV-2 OMICRON BINDING PROTEINS AND METHODS OF USE THEREOF

(71) Applicant: Regents of the University of Minnesota, Minneapolis, MN (US)

(72) Inventors: Frank Jonathan Lee, Minneapolis, MN (US); Gang Ye, Minneapolis, MN (US); Fan Bu, Minneapolis, MN (US)

(21) Appl. No.: 19/383,941

(22) Filed: Nov. 10, 2025

Related U.S. Application Data
(60) Provisional application No. 63/718,914, filed on Nov. 11, 2024.

(51) Int. Cl. C07K 16/104; A61K 39/00; A61P 31/14

(52) U.S. Cl. CPC C07K 16/104; A61P 31/14; A61K 2039/505; C07K 2317/24; C07K 2317/31; C07K 2317/565; C07K 2317/569; C07K 2317/72; C07K 2317/76; C07K 2317/92; C07K 2319/40

(57) ABSTRACT

Certain embodiments of the invention provide isolated anti-Omicron sdAbs, as well polypeptides and protein molecules comprising such sdAbs. Certain embodiments of the invention also provide methods of using these sdAbs, and protein molecules (e.g., monospecific or bispecific binder) for treating or preventing a SARS-CoV-2 Omicron infection.

### CROSS-REFERENCE TO RELATED APPLICATION

This application claims priority to U.S. Provisional Application No. 63/718,914 that was filed on Nov. 11, 2024. The entire content of the application referenced above is hereby incorporated by reference herein.

### GOVERNMENT FUNDING

This invention was made with government support under AI089728, AI157975, and AI171954 awarded by the National Institutes of Health. The government has certain rights in the invention.

### SEQUENCE LISTING

The instant application contains a Sequence Listing that has been submitted in XML format via Patent Center and is hereby incorporated by reference in its entirety. Said XML copy, created on Dec. 18, 2025, is named 09531_600US1_SL.xml and is 46,056 bytes in size.

### BACKGROUND OF THE INVENTION

Omicron subvariants of SARS-CoV-2 continue to circulate among human populations, posing a threat to global health. Since Omicron's emergence in the fall of 2021, new Omicron subvariants have continually appeared. It is likely that Omicron will persist in human populations long-term, necessitating ongoing interventions, including vaccines and antiviral therapeutics. Although SARS-CoV-2 vaccines are available, breakthrough infections are common. While several anti-SARS-CoV-2 therapeutics exist, current therapeutics have limitations and drawbacks. In addition, relying on a single drug increases the risk of viral escape mutations. Thus, novel anti-Omicron therapeutics are needed to effectively control Omicron.

### SUMMARY OF THE INVENTION

Certain embodiments of the invention provide a binder protein as described herein.

Certain embodiments of the invention provide an isolated anti-SARS-CoV-2 binder protein comprising one or more CDRs selected from the group consisting of:
(a) a CDR1 comprising an amino acid sequence having the amino acid sequence of TASGIALHX₁X₂ (SEQ ID NO:25), wherein X₁ is T, S, or H, and X₂ is H, K, or L;
(b) a CDR2 comprising an amino acid sequence having the amino acid sequence of ISSGDGTT (SEQ ID NO:3); and
(c) a CDR3 comprising an amino acid sequence having the amino acid sequence of DPX₃X₄VCHSGSYYYTDDDFYY (SEQ ID NO:26), wherein X₃ is G, S, or absent, and X₄ is A, G, or absent.

Certain embodiments of the invention provide an isolated anti-SARS-CoV-2 binder protein (e.g., monospecific binder or bispecific binder) comprising:
(1) one or more CDRs selected from the group consisting of:
   (a) a CDR1 comprising an amino acid sequence having at least 80% sequence identity to the amino acid sequence of TASGIALHTH (SEQ ID NO:2);
   (b) a CDR2 comprising an amino acid sequence having at least 80% sequence identity to the amino acid sequence of ISSGDGTT (SEQ ID NO:3); and
   (c) a CDR3 comprising an amino acid sequence having at least 80% sequence identity to the amino acid sequence of DPGAVCHSGSYYYTDDDFYY (SEQ ID NO:4); and/or
(2) one or more CDRs selected from the group consisting of:
   (a) a CDR1 comprising an amino acid sequence having at least 80% sequence identity to the amino acid sequence of TASGIALHSK (SEQ ID NO:13) or TASGIALHHL (SEQ ID NO:18);
   (b) a CDR2 comprising an amino acid sequence having at least 80% sequence identity to the amino acid sequence of ISSGDGTT (SEQ ID NO:3); and
   (c) a CDR3 comprising an amino acid sequence having at least 80% sequence identity to the amino acid sequence of DPSGVCHSGSYYYTDDDFYY (SEQ ID NO:14) or DPVCHSGSYYYTDDDFYY (SEQ ID NO:19).

Certain embodiments of the invention provide an isolated anti-SARS-CoV-2 (e.g., Omicron or variant thereof) binder protein that comprises one or more complementarity determining regions (CDRs) selected from the group consisting of:
(a) a CDR1 comprising an amino acid sequence having at least 80% sequence identity to the amino acid sequence of any one of TASGIALHTH (SEQ ID NO:2), TASGIALHSK (SEQ ID NO: 13), or TASGIALHHL (SEQ ID NO:18);
(b) a CDR2 comprising an amino acid sequence having at least 80% sequence identity to the amino acid sequence of ISSGDGTT (SEQ ID NO:3); and
(c) a CDR3 comprising an amino acid sequence having at least 80% sequence identity to the amino acid sequence of any one of DPGAVCHSGSYYYTDDDFYY (SEQ ID NO:4), DPSGVCHSGSYYYTDDDFYY (SEQ ID NO:14), or DPVCHSGSYYYTDDDFYY (SEQ ID NO:19).

Certain embodiments of the invention provide a sdAb-Fc fusion protein comprising an isolated anti-SARS-CoV-2 sdAb as described herein operably linked to an Fc domain amino acid sequence.

Certain embodiments of the invention provide a method for treating or preventing a SARS-CoV-2 (e.g., Omicron or variant thereof) infection in a mammal, comprising administering an effective amount of an isolated anti-SARS-CoV-2 binder protein as described herein, to the mammal.

Certain embodiments of the invention provide a composition comprising isolated anti-SARS-CoV-2 binder protein as described herein, and a carrier.

Certain embodiments of the invention provide an isolated anti-SARS-CoV-2 binder protein as described herein, for the prophylactic or therapeutic treatment of a SARS-CoV-2 infection (e.g., Omicron or variant thereof).

Certain embodiments of the invention provide an isolated anti-SARS-CoV-2 binder protein as described herein, for use in medical therapy.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1D. Binding kinetics between Nanosota-9 and Omicron spike ectodomains as measured by surface plasmon resonance (SPR). Each of the recombinant spike ectodomains from Omicron subvariants ((FIG. 1A) BA.5, (FIG. 1B) XBB.1.5, and (FIG. 1C) JN.1 respectively) was immobilized on a CM5 sensor chip. His-tagged Nanosota-9 (Nanosota-9-His) was injected at different concentrations. The resulting data were analyzed using Biacore Evaluation Software. (FIG. 1D) Competition SPR analysis. The JN.1 spike ectodomain was immobilized on two CM5 sensor chips. Fc-tagged Nanosota-9 (Nanosota-9-Fc) was injected onto the first chip, while the second chip received only the running buffer. Subsequently, a mixture of recombinant human ACE2 and Nanosota-9-Fc was added to the first chip, and only ACE2 was added to the second chip. Sensorgrams from both chips were overlaid for comparison. The lack of change in the resonance signal after ACE2 injection from the Nanosota-9-Fc-bound JN.1 spike ectodomain indicated that ACE2 could not displace Nanosota-9-Fc from binding to the JN.1 spike ectodomain.

FIGS. 2A-2E. Neutralization potency of Nanosota-9 against Omicron subvariants in vitro and in vivo. (FIG. 2A) Efficacy of Nanosota-9-Fc in neutralizing Omicron pseudoviruses. Retroviruses pseudotyped with each of the Omicron spike proteins (BA.5, XBB.1.5, and JN.1) were used to enter human ACE2-expressing cells in the presence of Nanosota-9-Fc at different concentrations. Entry efficiency was measured by the luciferase signal accompanying entry. Nonlinear regression was performed using a log (inhibitor) versus response curve. The efficacy of the nanobody was expressed as the concentration capable of neutralizing pseudovirus entry by 50% (i.e., IC₅₀). Data are the mean±SEM (n=3). (FIG. 2B) Efficacy of Nanosota-9-Fc in neutralizing live infectious Omicron in vitro. Each of the Omicron subvariants infected permissive Vero cells in the presence of Nanosota-9-Fc at different concentrations. Cell viability following 96 hours of incubation was determined using Neutral Red assay (Sigma-Aldrich). Nonlinear regression was performed using a log (inhibitor) versus response curve. The efficacy of Nanosota-9-Fc against each of the Omicron subvariants was calculated and expressed as the concentration capable of reducing the virus induced Cytopathic effect (CPE) by 50% (IC₅₀) compared to control serum-exposed virus. Data are the mean±SEM (n=4). (FIG. 2C)-(FIG. 2E) Efficacy of Nanosota-9-Fc in neutralizing live infectious Omicron in mice. Nanosota-9-Fc was administered at a dosage of 10 mg/kg body weight and 4 hours post-challenge. Mice were challenged via intranasal inoculation with each of the Omicron subvariants ((FIG. 2C) BA.5, (FIG. 2D) XBB.1.5, and (FIG. 2E) JN.1). In the treatment group (n=5), mice received Nanosota-9-Fc via intraperitoneal injection. In the control group (n=5), mice were administered PBS buffer. Virus titers in the mouse lungs on day 2 post-challenge were measured. The detection limit of the lung virus titer measurements is indicated by a line. Comparisons of lung virus titers between the control and treatment groups were performed using an unpaired two-tailed Student's t-test. Error bars represent SEM. **p<0.01; ****p<0.0001.

FIGS. 3A-3D. Overall structure of Omicron spike ectodomains complexed with Nanosota-9. (FIG. 3A) Cryo-EM structures of BA.5 spike ectodomain complexed with Nanosota-9. Three spike-bound Nanosota-9 molecules are colored in yellow. The three RBDs are colored in cyan (core region) and magenta (RBM), with one RBD in the standing up position and the other two in the lying down position. (FIG. 3B) Cryo-EM structures of JN.1 spike ectodomain complexed with Nanosota-9. (FIG. 3C) Footprints of Nanosota-9 and ACE2 on the BA.5 RBD. Nanosota-9-binding RBD residues, ACE2-binding RBD residues, and dual-binding RBD residues are colored as indicated. The Nanosota-9 epitope heavily overlaps with the ACE2-binding site on the RBD, blocking receptor binding to the RBD and thereby neutralizing Omicron entry. (FIG. 3D) Footprints of Nanosota-9 and ACE2 on the JN.1 RBD.

FIGS. 4A-4D. Nanosota-9 binds to Omicron spike protein using a novel crosslinking mechanism. (FIG. 4A) Two Nanosota-9 molecules crosslink two RBDs together. The 2:2 binding mode creates three interfaces: a major interface between Nanosota-9 and the RBM of one RBD, a minor interface between Nanosota-9 and the core of another RBD, and an additional interface between the two Nanosota-9 molecules. (FIG. 4B) Detailed interactions at the major interface between Nanosota-9 and the RBM of one RBD. (FIG. 4C) Detailed interactions at the minor interface between Nanosota-9 and the core of another RBD. (FIG. 4D) Detailed interactions at the additional interface between the two Nanosota-9 molecules. Dotted lines indicate hydrogen bonds or salt bridges. Double arrows indicate hydrophobic stacking interactions. The crosslinking mechanisms and the three interfaces stabilize the lying down RBD, preventing it from binding to the ACE2 receptor and enhancing Nanosota-9's anti-Omicron potency.

FIGS. 5A-5H. Evolution of Omicron RBDs within the Nanosota-9 binding epitopes. (FIG. 5A) Sequence alignment of the Nanosota-9-binding RBD residues among six Omicron subvariants: BA.1, BA.5, XBB.1.5, JN.1, KP.2, and KP.3. RBD residues in direct contact with Nanosota-9 are colored in blue (conserved among Omicron subvariants) or red (mutated among Omicron subvariants). Two RBD residues that are not in direct contact with Nanosota-9 but underwent mutations in KP.2 and KP.3 subvariants are labeled in bold black. Mutated residues among Omicron subvariants are boxed. Asterisks indicate positions with a single, fully conserved residue. Colons indicate positions with strongly conserved residues. Periods indicate positions with weakly conserved residues. FIG. 5A discloses SEQ ID NOs: 33-38, respectively, in order of appearance. (FIG. 5B)-(FIG. 5D) Structural details of Nanosota-9-binding RBD residues that underwent mutations among the three Omicron subvariants, BA.5, XBB.1.5, and JN.1. (FIG. 5E) Structural details of Nanosota-9-binding RBD residue 489 that underwent a mutation in Omicron subvariant BA.1. (FIG. 5F)-(FIG. 5G) Mapping of RBD residues that underwent mutations in Omicron subvariants KP.2 and KP.3. (FIG. 5H) Structural details of Nanosota-9-binding RBD residue 489 that underwent a mutation in Omicron subvariant KP.3.

FIGS. 6A-6B. Biochemical assessment of the anti-Omicron spectrum of Nanosota-9. (FIG. 6A) Flow cytometry analysis of the interaction between Nanosota-9-Fc and cell surface-expressed spike proteins from major Omicron subvariants. The flow cytometry signal, expressed as mean fluorescence intensity (MFI), was measured using PE anti-Fc tag antibodies, which target the Fc-tagged Nanosota-9 protein bound to spike-positive cells. The control group consisted of cells transfected with an empty vector. Data are presented as mean±SEM (n=3). Statistical differences between the control group and each experimental group were analyzed using a two-tailed Student's t-test. ****p<0.0001. (FIG. 6B) Neutralization efficacy of Nanosota-9-Fc against pseudoviruses of major Omicron subvariants. The assay was conducted as described in FIG. 2A.

FIG. 7. Binding interactions between Nanosota-9-Fc and Omicron spike ectodomains as measured by ELISA. ELISA plates were coated with one of the recombinant Omicron spike ectodomains and then incubated with Nanosota-9-Fc. Spike-bound Nanosota-9-Fc was detected using anti-human Fc antibody.

FIG. 8. Flow chart of cryo-EM image processing and 3D reconstruction for the complex of BA.5 spike ectodomain and Nanosota-9. Representative raw cryo-EM image and 2D classes are presented. 3D refinements using all the particles from good 3D classes generated a 3.01 Å map. Further local refinement improved the density for the bound nanobody. The angular distribution plot, final maps, half-map FSC curves and accompanying local resolution illustrations are enclosed in the dashed black boxes.

FIG. 9. Flow chart of cryo-EM image processing and 3D reconstruction for the complex of JN.1 spike ectodomain and Nanosota-9. Representative raw cryo-EM image and 2D classes are presented. 3D refinements using all the particles from good 3D classes generated a 2.99 Å map. Further local refinement improved the density for the bound nanobody. The angular distribution plot, final maps, half-map FSC curves and accompanying local resolution illustrations are enclosed in the dashed black boxes.

FIG. 10. Cryo-EM densities of the three interfaces formed among two Nanosota-9 molecules and two Omicron RBDs. These three interfaces are: the major interface between the standing-up RBD and Nanosota-9, the minor interface between the lying-down RBD and Nanosota-9, and the additional interface between two Nanosota-9 molecules.

FIGS. 11A-11D. Detailed interactions between the BA.5 spike ectodomain and Nanosota-9. This figure was prepared in the same way as FIG. 4, except that the BA.5 spike ectodomain was used instead of the JN. I spike ectodomain. (FIG. 11A) Two Nanosota-9 molecules crosslink two RBDs together. The 2:2 binding mode creates three interfaces: a main interface between Nanosota-9 and the RBM of one RBD, a minor interface between Nanosota-9 and the core of another RBD, and an additional interface between the two Nanosota-9 molecules. (FIG. 11B) Detailed interactions at the main interface between Nanosota-9 and the RBM of one RBD. (FIG. 11C) Detailed interactions at the minor interface between Nanosota-9 and the core of another RBD. (FIG. 11D) Detailed interactions at the additional interface between the two Nanosota-9 molecules. Dotted lines indicate hydrogen bonds or salt bridges. Double arrows indicate hydrophobic stacking interactions. The crosslinking mechanisms and the three interfaces stabilize the lying down RBDs, preventing them from binding to the ACE2 receptor and enhancing Nanosota-9's anti-Omicron potency.

FIGS. 12A-12B. Surface presentation of the JN.1 spike ectodomain complexed with Nanosota-9. (FIG. 12A) Side view of the structure. (FIG. 12B) Top view of the structure.

FIG. 13. Flow cytometry data showing the interactions between Nanosota-9-Fc and cell surface-expressed spike proteins from major Omicron subvariants.

FIGS. 14A-14B. The Nanosota-9 binding site is inaccessible to human antibodies when the RBD is in a lying-down position. (FIG. 14A) Docking of a human antibody to the Nanosota-9 binding site on the Omicron spike. The antigen-binding domains of a human antibody (PDB 7B30) were docked onto the structure of the JN.1 spike ectodomain/Nanosota-9 complex by structurally aligning the heavy-chain (HC) antigen-binding domain of the human antibody and Nanosota-9. Nanobodies and heavy-chain antigen-binding domains of human antibodies are evolutionarily and functionally related. The blue circle indicates a clash between the light-chain (LC) antigen-binding domain of the human antibody and the lying-down RBD, suggesting that human antibodies cannot access the Nanosota-9 binding site on Omicron spikes. (FIG. 14B) Overlay of the structures of human antibodies and Nanosota-9 all bound to the Omicron RBD. The structures of the Omicron spike ectodomains complexed with Fabs from human antibodies (PDB IDs labeled in parentheses) were overlaid with the JN.1 spike ectodomain/-Nanosota-9 complex by structurally aligning their spike RBDs. Only one human antibody (PDB 7TAT) shares an overlapping epitope with Nanosota-9 on the lying-down RBD, but it clashes with a standing-up RBD, as indicated by the blue circle. Structural alignments were performed using PyMol.

FIG. 15. Overlay of the structures of four nanobodies on the SARS-CoV-2 RBD. The structures of the prototypic SARS-CoV-2 spike each complexed with Nanosota-2, -3, or -4 (PDBs: 8G72, 8G74, and 8G75) were overlaid with the structure of the JN.1 spike complexed with Nanosota-9 through structural alignment of their spike RBDs. Nanosota-9 clashes with Nanosota-2 and -3, but not with Nanosota-4. Structural alignments were performed using PyMol.

FIGS. 16A-16C. A single Q493E mutation in the Omicron JN.1 RBD abolishes Nanosota-9 binding. (FIG. 16A) Evolution at RBD residue 493 across SARS-CoV-2 variants and Omicron subvariants. (FIG. 16B) Structure of the JN.1 RBD bound to Nanosota-9A (PDB 9CO8). The JN.1 RBD core is shown in cyan, the receptor-binding motif (RBM) in magenta, and Nanosota-9A in yellow. Omicron RBD numbering is aligned to the prototypic SARS-CoV-2 RBD. Residue 493 is shown as red sticks. (FIG. 16C) Detail of the JN.1 RBD and Nanosota-9A interface. Gln493 in the JN.1 RBD forms two hydrogen bonds: one with the Thr31 side chain and one with the main-chain carbonyl oxygen of His32 in Nanosota-9A. The Q493E mutation introduces a clash with the His32 main-chain carbonyl, abolishing Nanosota-9A binding to the JN.1 RBD. Four nanobody residues near residue 493 (Thr31, His32, Gly 101, and Ala102) were selected for randomization in the structure-guided in vitro evolution of Nanosota-9A.

FIG. 17. Flowchart of structure-guided in vitro evolution of Nanosota-9A to overcome the Q493E escape mutation in the JN.1 RBD. The workflow is shown along with the four nanobody residues selected for randomization, before (Nanosota-9A) and after the procedure (Nanosota-9B).

FIGS. 18A-18D. Functional characterization of Nanosota-9B. (FIG. 18A) Binding interactions between Nanosota-9B-Fc (Fc-tagged Nanosota-9B) and Omicron spike ectodomains were evaluated by ELISA. ELISA plates were coated with His-tagged Omicron spike ectodomain (from the JN.1 or KP.3 subvariant), followed by addition of Nanosota-9B-Fc. Binding was detected using anti-Fc-tag antibodies. (FIG. 18B) Efficacy of Nanosota-9B-Fc in neutralizing Omicron pseudoviruses. Retroviruses pseudotyped with Omicron spike (from JN.1, KP.2, or KP.3) were used to infect human ACE2-expressing cells in the presence of Nanosota-9B-Fc at various concentrations. Entry efficiency was measured by luciferase signal. The efficacy of Nanosota-9B-Fc against each pseudovirus type was expressed as the concentration capable of neutralizing pseudovirus entry by 50% (IC₅₀). Error bars represent SEM (n=3). Each experiment was repeated at least three times with similar results. (FIG. 18C) and (FIG. 18D) Efficacy of Nanosota-9B-Fc in neutralizing authentic JN.1 (FIG. 18C) and KP.3 (FIG. 18D) in mice. C57BL/6 mice were challenged by intranasal inoculation with Omicron subvariants. Nanosota-9B-Fc was administered at 10 mg/kg body weight 4 hours post-infection. C57BL/6 mice were challenged by intranasal inoculation with Omicron. In the treatment group (n=4 for each virus), mice received Nanosota-9B-Fc intraperitoneally. In the control group (n=4 for JN.1 and n=3 for KP.3), mice received PBS. Lung virus titers on day 2 post-infection were measured by focus-forming assay (FFA). Comparisons of lung virus titers between control and treatment groups were performed using an unpaired two-tailed Student's t-test. ***p<0.001; ***p<0.0001.

FIGS. 19A-19B. Structure of the KP.3 spike ectodomain complexed with Nanosota-9B. (FIG. 19A) Overall cryo-EM structure of the KP.3 spike ectodomain complexed with Nanosota-9B. The KP.3 spike ectodomain is shown in gray, with the three RBDs in cyan. Two RBDs are in the standing-up conformation, and one is in the lying-down conformation. Nanosota-9B (yellow) binds all three RBDs. (FIG. 19B) Detailed view of the KP.3 RBD and Nanosota-9B interface. The newly evolved nanobody residues Ser31 and Lys32 form a hydrogen bond and a salt bridge, respectively, with Glu493 of the KP.3 RBD, overcoming the Q493E mutation. Another newly evolved nanobody residue, Ser101, forms a hydrogen bond with Tyr497 of the KP.3 RBD, further stabilizing the RBD-nanobody complex.

FIGS. 20A-20C. Construction and functional characterization of the bispecific nanobody Nanosota-9A/9B-Fc. A bispecific nanobody, Nanosota-9A/9B-Fc, was generated by fusing Nanosota-9A and Nanosota-9B to a human Fc domain. (FIG. 20A) Efficacy of Nanosota-9A/9B-Fc in neutralizing Omicron pseudoviruses. The assay was performed as in FIG. 18B. (FIG. 20B) Efficacy of Nanosota-9A/9B-Fc in neutralizing authentic Omicron in vitro. Each Omicron subvariant infected Vero cells expressing human ACE2 and TMPRSS2 in the presence of Nanosota-9A/9B-Fc at different concentrations. Infection efficiency was determined by the remaining cell viability after 96 hours (lower viability indicates higher infection). Efficacy for each subvariant is expressed as the IC₅₀, the concentration that reduces virus-induced cytopathic effect by 50% relative to serum-exposed virus controls. Data are mean±SEM (n=4). (FIG. 20C) Efficacy of Nanosota-9A/9B-Fc in neutralizing authentic Omicron (JN.1, KP.2, and KP.3) in mice. The experiment followed FIG. 18C and FIG. 18D, except lung virus titers on day 2 post-infection were measured by TCID₅₀ assay. Treatment groups, n=5; control groups, n=4. Comparisons of lung virus titers between the control and treatment groups were performed using an unpaired two-tailed Student's t-test. **p<0.01.

FIG. 21. Phylogenetic tree of representative Omicron subvariants [35].

FIGS. 22A-22C. Nanosota-9A binds the JN.1 spike ectodomain via a crosslinking mechanism [32]. (FIG. 22A) Previously determined cryo-EM structure of the JN.1 spike ectodomain complexed with Nanosota-9A (PDB 9CO8). (FIG. 22B) Two Nanosota-9A molecules crosslink two JN.1 RBDs (one standing up and one lying down). Each RBD contains a core and a receptor-binding motif (RBM). This 2:2 binding mode creates a main interface between Nanosota-9A and the RBM of one RBD and a minor interface between Nanosota-9A and the core of the other RBD. Human antibodies cannot fit into the Nanosota-9A binding epitope on the lying-down RBD. (FIG. 22C) Neutralizing potency of Nanosota-9A-Fc (Fc-tagged Nanosota-9A) against different Omicron subvariants.

FIGS. 23A-23B. Characterization of two Nanosota-9B candidates. (FIG. 23A) Structure-guided evolution of Nanosota-9A yielded two candidates (Nanosota-9B-1 and Nanosota-9B-2), both of which bind the KP.3 spike ectodomain. They differ at four residues targeted for randomization. (FIG. 23B) Binding of each candidate to the KP.3 spike ectodomain was assessed by ELISA. Plates were coated with His-tagged KP.3 ectodomain, incubated with HA-tagged Nanosota-9B, and binding was detected with anti-HA antibodies. Nanosota-9B-1 showed stronger binding and was therefore selected as Nanosota-9B for further characterization.

FIG. 24. Flowchart of cryo-EM image processing and 3D reconstruction for the KP.3 spike ectodomain/Nanosota-9B complex. Representative raw micrographs and 2D class averages are shown. 3D refinements using particles from high-quality 3D classes yielded a 3.06 Å map. Subsequent local refinement improved the density of the bound nanobody. The angular distribution plot, final maps, half-map FSC curves, and local-resolution estimates are enclosed in the dashed black boxes.

FIGS. 25A-25C. Nanosota-9A/9B-Fc shows low neutralizing potency against the Omicron subvariant XFG. (FIG. 25A) Neutralization of Omicron pseudoviruses by Nanosota-9A/9B-Fc, performed as in FIG. 18B. (FIG. 25B) Mapping of RBD residues mutated within or near the Nanosota-9 binding epitopes. (FIG. 25C) Sequence alignment of Nanosota-9-contacting RBD residues across Omicron subvariants. Residues in direct contact with Nanosota-9 are colored blue (conserved) or red (mutated). RBD residues that do not directly contact Nanosota-9 but mutated from JN.1 to KP.2/KP.3/XFG are shown in bold black. Asterisks denote fully conserved positions, colons indicate strong conservation, and periods indicate weak conservation. FIG. 25C discloses SEQ ID NOs: 39, 34, 40-42, 36-38, and 43, respectively, in order of appearance.

### DETAILED DESCRIPTION

Omicron continues to evolve and spread among humans, making the development of effective and broad-spectrum treatments a top priority for global health. Single-domain antibodies show great promise due to their small size and ability to target conserved, hidden regions on the virus. As described herein, novel anti-Omicron binder proteins (e.g., Nanosota-9, also referred to as Nanosota-9A) are identified herein, which effectively inhibit most major Omicron subvariants. Further cryo-EM analysis revealed a unique structural mechanism by which the binder protein neutralizes Omicron. Nanosota-9 binds to the receptor-binding domain (RBD) of Omicron spike proteins, blocking receptor binding. Furthermore, two Nanosota-9 molecules could crosslink two RBDs of the trimeric spike protein, enhancing its anti-Omicron potency. One of the two binding sites on the RBD that Nanosota-9 targets significantly overlap with the receptor-binding site and is relatively conserved, while the other site is fully conserved, expanding its anti-Omicron spectrum. Conventional antibodies cannot access the same binding sites on the spike protein as Nanosota-9, highlighting its unique neutralization mechanism. The anti-Omicron binder proteins described herein have strong potential as superior anti-Omicron therapeutics.

In addition, SARS-CoV-2 continues to accumulate spike mutations that erode the efficacy of antibody therapeutics. The Q493E mutation in the spike RBD, present in more recent Omicron subvariants, enables escape from many antibodies and nanobodies, including Nanosota-9A nanobody, which neutralizes Omicron JN.1 (Q493) but not KP.3 (E493). To address this, we applied a structure-guided in vitro evolution strategy to engineer Nanosota-9A, generating Nanosota-9B, which binds the KP.3 RBD with high affinity but shows reduced binding to JN.1. To regain breadth, we engineered a bispecific nanobody combining Nanosota-9A and -9B. This construct effectively neutralizes both JN.1 and KP.3 in infection assays. These results described herein also establish an “update, don't rediscover” antiviral paradigm: reuse validated nanobodies and apply structure-guided engineering to overcome variant escape. This strategy offers a practical path to maintain therapeutic coverage as the virus evolves, enabling more efficient use of research resources and rapid responses to emerging variants.

The terms “nanobody” and “single-domain antibody (sdAb)” are used interchangeably herein. As used herein, the term “nanobody” or “single-domain antibody” refers to a single monomeric variable antibody domain comprising three complementarity-determining regions (CDRs including CDR1, CDR2, CDR3) and four framework regions (FRs including FR1, FR2, FR3, FR4), such as a VHH, a humanized VHH or a camelized VH (such as a camelized human VH) or generally a sequence optimized VHH (such as e.g., optimized for chemical stability and/or solubility, maximum overlap with known human framework regions and maximum expression), which is capable of binding to a specific antigen. The terms “nanobody” and “single-domain antibody” are used herein in its broadest form to include variants that may have various amino acid substitutions (e.g., conservative substitutions) and also functional “fragment” or “antigen binding fragment” of the sdAb as long as the fragment retains binding to the specific antigen.

As used herein, the term “anti-SARS-CoV-2 binder protein” refers to a protein having binding affinity for a SARS-CoV-2 viral antigen (e.g., SARS-CoV-2 Omicron or its variant spike protein). Such a protein may comprise or consist of a sdAb as described herein, or an antigen binding fragment thereof. In certain embodiments, an anti-SARS-CoV-2 binder protein comprises a sdAb as described herein. In certain embodiments, an anti-SARS-CoV-2 binder protein consists of a sdAb as described herein. In certain embodiments, an anti-SARS-CoV-2 binder protein comprises an antigen binding fragment of a sdAb as described herein.

Accordingly, certain embodiments of the invention provide an anti-SARS-CoV-2 binder protein (e.g., targeting a SARS-CoV-2 Omicron spike protein) as described herein. In certain embodiments, the anti-SARS-CoV-2 binder protein comprises a sdAb, and optionally, one or more polypeptide tags, wherein the sdAb is operably linked to the one or more polypeptide tags (e.g., a monomeric sdAb with an optional His tag and/or HA tag). In certain embodiments, the anti-SARS-CoV-2 binder protein comprises two sdAb-Fc fusion proteins that are dimerized via the Fc domain. In certain embodiments, an anti-SARS-CoV-2 binder protein comprises or consists of a polypeptide sequence as described in Example 1, Example 2, or in Table A.

In some embodiments, the binder protein (e.g., an anti-SARS-CoV-2 sdAb or sdAb-Fc fusion) comprises: (1) one or more complementarity determining region (CDR) sequences as described herein; and/or (2) a heavy chain variable region (VHH) sequence as described herein (e.g., as described in Table A).

In some embodiments, the binder protein comprises one, two, or three CDRs selected from Table A as described herein. In some embodiments, the binder protein comprises two, or three CDRs selected from Table A. In some embodiments, the binder protein comprises three CDRs (CDR1, CDR2, and CDR3) as described herein. In some embodiments, the binder protein (e.g., a bispecific binder) comprises four, five, or six CDRs as described herein.

Accordingly, certain embodiments provide a binder protein comprising one or more CDRs selected from the group consisting of:
(a) a CDR1 comprising an amino acid sequence having the amino acid sequence of TASGIALHX₁X₂ (SEQ ID NO:25), wherein X₁ is T, S, or H, and X₂ is H, K, or L;
(b) a CDR2 comprising an amino acid sequence having the amino acid sequence of ISSGDGTT (SEQ ID NO:3); and
(c) a CDR3 comprising an amino acid sequence having the amino acid sequence of DPX₃X₄VCHSGSYYYTDDDFYY (SEQ ID NO:26), wherein X₃ is G, S, or absent, and X₄ is A, G, or absent.

In certain embodiments, the binder protein comprises:
(a) a CDR1 comprising an amino acid sequence having the amino acid sequence of TASGIALHX₁X₂ (SEQ ID NO:25), wherein X₁ is T, S, or H, and X₂ is H, K, or L;
(b) a CDR2 comprising an amino acid sequence having the amino acid sequence of ISSGDGTT (SEQ ID NO:3); and
(c) a CDR3 comprising an amino acid sequence having the amino acid sequence of DPX₃X₄VCHSGSYYYTDDDFYY (SEQ ID NO:26), wherein X₃ is G, S, or absent, and X₄ is A, G, or absent.

In certain embodiments, X₁ is T, and X₂ is H.

In certain embodiments, X₃ is G, and X₄ is A.

In certain embodiments, X₁ is S, and X₂ is K.

In certain embodiments, X₃ is S, and X₄ is G.

In certain embodiments, X₁ is H, and X₂ is L.

In certain embodiments, X₃ is absent, and X₄ is absent.

In certain embodiment, the binder protein comprises:
(a) a CDR1 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to the amino acid sequence of any one of SEQ ID NO:2, 13 or 18;
(b) a CDR2 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to the amino acid sequence of SEQ ID NO:3; and/or
(c) a CDR3 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to the amino acid sequence of any one of SEQ ID NO:4, 14 or 19.

In some embodiments, the binder protein comprises one or more CDRs selected from the group consisting of:
(a) a CDR1 comprising an amino acid sequence having at least 80% or 90% sequence identity to the amino acid sequence of any one of TASGIALHTH (SEQ ID NO:2), TASGIALHSK (SEQ ID NO: 13), or TASGIALHHL (SEQ ID NO:18);
(b) a CDR2 comprising an amino acid sequence having at least 80% or 90% sequence identity to the amino acid sequence of ISSGDGTT (SEQ ID NO:3); and
(c) a CDR3 comprising an amino acid sequence having at least 80% or 90% sequence identity to the amino acid sequence of any one of DPGAVCHSGSYYYTDDDFYY (SEQ ID NO:4), DPSGVCHSGSYYYTDDDFYY (SEQ ID NO:14), or DPVCHSGSYYYTDDDFYY (SEQ ID NO: 19).

In some embodiments, the binder protein comprises three CDRs selected from the group consisting of:
(a) a CDR1 comprising an amino acid sequence having at least 90% sequence identity to the amino acid sequence of any one of TASGIALHTH (SEQ ID NO:2), TASGIALHSK (SEQ ID NO: 13), or TASGIALHHL (SEQ ID NO:18);
(b) a CDR2 comprising the amino acid sequence of ISSGDGTT (SEQ ID NO:3); and
(c) a CDR3 comprising the amino acid sequence of any one of

(SEQ ID NO: 4)
DPGAVCHSGSYYYTDDDFYY,
(SEQ ID NO: 14)
DPSGVCHSGSYYYTDDDFYY,
or
(SEQ ID NO: 19)
DPVCHSGSYYYTDDDFYY.

In some embodiments, the binder protein comprises three CDRs selected from the group consisting of:
(a) a CDR1 comprising the amino acid sequence of TASGIALHTH (SEQ ID NO:2), TASGIALHSK (SEQ ID NO:13), or TASGIALHHL (SEQ ID NO:18);
(b) a CDR2 comprising an amino acid sequence having at least 90% sequence identity to the amino acid sequence of ISSGDGTT (SEQ ID NO:3); and
(c) a CDR3 comprising the amino acid sequence of DPGAVCHSGSYYYTDDDFYY (SEQ ID NO: 4), DPSGVCHSGSYYYTDDDFYY (SEQ ID NO: 14), or DPVCHSGSYYYTDDDFYY (SEQ ID NO:19).

In some embodiments, the binder protein comprises three CDRs selected from the group consisting of:
(a) a CDR1 comprising the amino acid sequence of TASGIALHTH (SEQ ID NO:2), TASGIALHSK (SEQ ID NO:13), or TASGIALHHL (SEQ ID NO:18);
(b) a CDR2 comprising the amino acid sequence of ISSGDGTT (SEQ ID NO:3); and
(c) a CDR3 comprising an amino acid sequence having at least 90% sequence identity to the amino acid sequence of DPGAVCHSGSYYYTDDDFYY (SEQ ID NO:4), DPSGVCHSGSYYYTDDDFYY (SEQ ID NO:14), or DPVCHSGSYYYTDDDFYY (SEQ ID NO: 19).

In certain embodiments, the anti-SARS-CoV-2 binder protein as described herein comprises one or more CDRs selected from the group consisting of:
(a) a CDR1 comprising the amino acid sequence of SEQ ID NO:2, 13, or 18;
(b) a CDR2 comprising the amino acid sequence of SEQ ID NO:3; and
(c) a CDR3 comprising the amino acid sequence of SEQ ID NO:4, 14, or 19.

In certain embodiments, the anti-SARS-CoV-2 binder protein as described herein comprises:
(a) a CDR1 comprising the amino acid sequence of SEQ ID NO:2, 13, or 18;
(b) a CDR2 comprising the amino acid sequence of SEQ ID NO:3; and
(c) a CDR3 comprising the amino acid sequence of SEQ ID NO:4, 14, or 19.

Certain embodiments of the invention provide an anti-SARS-CoV-2 binder protein described herein comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to:

(SEQ ID NO: 1)
QVQLQESGGGLVQPGGSLRLSCTASGIALHTHATGWFRQAPGKEREGVS
CISSGDGTTYYEDSVEGRFTISRDNAKNTVYLQMNSLKLEDTAVYYCAA
DPGAVCHSGSYYYTDDDFYYRGQGTQVTVSS;
(SEQ ID NO: 12)
QVQLQESGGGLVQPGGSLRLSCTASGIALHSKATGWFRQAPGKEREGVS
CISSGDGTTYYEDSVEGRFTISRDNAKNTVYLQMNSLKLEDTAVYYCAA
DPSGVCHSGSYYYTDDDFYYRGQGTQVTVSS;
or
(SEQ ID NO: 17)
QVQLQESGGGLVQPGGSLRLSCTASGIALHHLATGWFRQAPGKEREGVS
CISSGDGTTYYEDSVEGRFTISRDNAKNTVYLQMNSLKLEDTAVYYCAA
DPVCHSGSYYYTDDDFYYRGQGTQVTVSS.

In certain embodiments, an anti-SARS-CoV-2 binder protein described herein comprises an amino acid sequence that has at least 85%, 90%, 95%, 97%, 99% or 100% sequence identity to SEQ ID NO:1, 12, or 17. In some embodiments, an anti-SARS-CoV-2 binder protein comprises the VHH amino acid sequence of SEQ ID NO:1, 12, or 17. In some embodiments, an anti-SARS-CoV-2 binder protein consists of the VHH amino acid sequence of SEQ ID NO:1, 12, or 17.

In some embodiments, an anti-SARS-CoV-2 binder protein (e.g., Nanosota-9A) comprises a CDR1 comprising the amino acid sequence of SEQ ID NO:2, a CDR2 comprising the amino acid sequence of SEQ ID NO:3, and a CDR3 comprising the amino acid sequence of SEQ ID NO:4. In some embodiments, an anti-SARS-CoV-2 binder protein comprises CDRs 1-3 consisting of the amino acid sequences of SEQ ID NOs: 2, 3, and 4, respectively.

In some embodiments, an anti-SARS-CoV-2 binder protein (e.g., Nanosota-9A) comprises an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:1. In some embodiments, an anti-SARS-CoV-2 binder protein comprises an amino acid sequence that 1) has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:1; and 2) comprises SEQ ID NO:2, SEQ ID NO:3 and SEQ ID NO: 4.

In some embodiments, an anti-SARS-CoV-2 binder protein (e.g., Nanosota-9B) comprises a CDR1 comprising the amino acid sequence of SEQ ID NO:13, a CDR2 comprising the amino acid sequence of SEQ ID NO:3, and a CDR3 comprising the amino acid sequence of SEQ ID NO:14. In some embodiments, an anti-SARS-CoV-2 binder protein comprises CDRs 1-3 consisting of the amino acid sequences of SEQ ID NOs: 13, 3, and 14, respectively.

In some embodiments, an anti-SARS-CoV-2 binder protein (e.g., Nanosota-9B) comprises an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:12. In some embodiments, an anti-SARS-CoV-2 binder protein comprises an amino acid sequence that 1) has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 12; and 2) comprises SEQ ID NO:13, SEQ ID NO:3 and SEQ ID NO:14.

In some embodiments, an anti-SARS-CoV-2 binder protein (e.g., Nanosota-9B-2) comprises a CDR1 comprising the amino acid sequence of SEQ ID NO: 18, a CDR2 comprising the amino acid sequence of SEQ ID NO:3, and a CDR3 comprising the amino acid sequence of SEQ ID NO:19. In some embodiments, an anti-SARS-CoV-2 binder protein comprises CDRs 1-3 consisting of the amino acid sequences of SEQ ID NOs: 18, 3, and 19, respectively.

In some embodiments, an anti-SARS-CoV-2 binder protein (e.g., Nanosota-9B-2) comprises an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:17. In some embodiments, an anti-SARS-CoV-2 binder protein comprises an amino acid sequence that 1) has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:17; and 2) comprises SEQ ID NO:18, SEQ ID NO:3 and SEQ ID NO:19.

In certain embodiments, a binder protein described herein is a broad-spectrum SARS-CoV-2 Omicron binding protein.

In certain embodiments, the binder protein specifically binds to SARS-CoV-2 Omicron and one or more variants (e.g., BA.5, XBB.1.5, or JN.1).

In certain embodiments, the binder protein binds to one or more (e.g., 2, 3, 4, 5, 6 or more) Omicron or its variants selected from the group consisting of SARS-CoV-2 Omicron BA.1, Omicron BA.5, Omicron XBB.1.5, Omicron JN.1, Omicron BA.2.75, Omicron BQ.1, Omicron EG.5, Omicron KP.2 and Omicron KP.3.

In certain embodiments, the binder protein binds to one or more (e.g., 2, 3, 4, 5, 6 or more) Omicron or its variants selected from the group consisting of SARS-CoV-2 Omicron BA.1, Omicron BA.5, Omicron XBB.1.5, Omicron JN.1, Omicron BA.2.75, Omicron BQ.1, Omicron EG.5, and Omicron KP.2.

In certain embodiments, the binder protein binds to one or more (e.g., 2, 3, 4, 5, 6 or more) Omicron or its variants selected from the group consisting of SARS-CoV-2 Omicron BA.5, Omicron XBB.1.5, Omicron JN.1, Omicron BA.2.75, Omicron BQ.1, Omicron EG.5, and Omicron KP.2.

In certain embodiments, the binder protein specifically binds to one or more (e.g., 2, 3, 4, or more) Omicron or its variants selected from the group consisting of Omicron BA.5, Omicron XBB.1.5, Omicron JN.1, Omicron KP.2, and Omicron KP.3.

In certain embodiments, the binder protein specifically binds to one or more (e.g., 2, or 3) Omicron or its variants selected from the group consisting of Omicron JN.1, Omicron KP.2, and Omicron KP.3.

“Broad-spectrum” as used herein refers to the ability of a binder protein to bind to two or more (e.g., 3, 4, 5, 6, or more) SARS-CoV-2 strains or variants. For example, in certain embodiments, a binder protein as described herein (e.g., sdAb, or sdAb-Fc) is capable of binding to 4, 5, 6 or more SARS-CoV-2 Omicron or variants. In certain embodiments, the two or more (e.g., 3, 4, 5, 6, or more) SARS-CoV-2 Omicron or variants are selected from the group consisting of consisting of SARS-CoV-2 Omicron BA.1, SARS-CoV-2 Omicron BA.5, Omicron XBB.1.5, Omicron JN.1, Omicron BA.2.75, Omicron BQ.1, Omicron EG.5, Omicron KP.2 and Omicron KP.3. The spike protein of SARS-CoV-2 Omicron are known in the art and described herein, for example, the GISAID accession numbers for S proteins (or gene) are described herein: EG.5 subvariant (GISAID: EPI_ISL_17524442), BA.1 subvariant (GISAID: EPI_ISL_6590782.2), BA.5 subvariant (GISAID: EPI_ISL_12954165), XBB.1.5 subvariant (GISAID: EPI_ISL_17774216), JN.1 subvariant (GISAID: EPI_ISL_17774216), KP.2 subvariant (GISAID: EPI_ISL_19214303), and KP.3 subvariant (GISAID: EPI_ISL_19214243); the spike genes of BA.2.75 (GISAID: EPI_ISL_13502529), and BQ.1 (GISAID: EPI_ISL_16609492).

In certain embodiments, the binder protein described herein specifically binds to SARS-CoV-2 Omicron or its variants (e.g., BA.5, XBB.1.5, or JN.1). In certain embodiments, a binder protein described herein is capable of specifically binding to the receptor binding domain (RBD) of a SARS-CoV-2 spike protein (e.g., Omicron or variant).

In certain embodiments, a binder protein described herein is capable of neutralizing (e.g., neutralize viral entry) one or more SARS-CoV-2 Omicron or variants. In certain embodiments, a binder protein described herein is capable of neutralizing two or more (e.g., 3, 4, 5, 6, or more) SARS-CoV-2 Omicron or variants.

In certain embodiments, the binder protein described herein has higher affinity for the receptor binding domain (RBD) of a SARS-CoV spike protein (e.g., RBD of SARS-CoV-2 Omicron or variant) than that of human angiotensin converting enzyme 2 (ACE2). In certain embodiments, the binder protein prevents ACE2 from binding to the RBD.

In certain embodiments, the binder protein's binding footprint on the RBD overlaps with ACE2's binding region on the RBD. In certain embodiments the binder protein has a footprint as described herein (e.g., binds one or more residues as described herein, see Example 1).

In certain embodiments, the binder protein is capable of binding to ten or more Omicron BA.5 spike protein RBD residues (e.g., 11, 12, 13, 14 or more residues) selected from the group consisting of Y444, L450, A479, G480, V481, C483, Y484, F485, Q488, S489, R493, T495, Y496, G497, and H500.

In certain embodiments, the binder protein is capable of binding to ten or more Omicron JN.1 spike protein RBD residues (e.g., 11, 12, 13 or 14 residues) selected from the group consisting of Y446, K480, G481, P482, C484, Y485, F486, Q489, S490, R494, T496, Y497, G498, and H501.

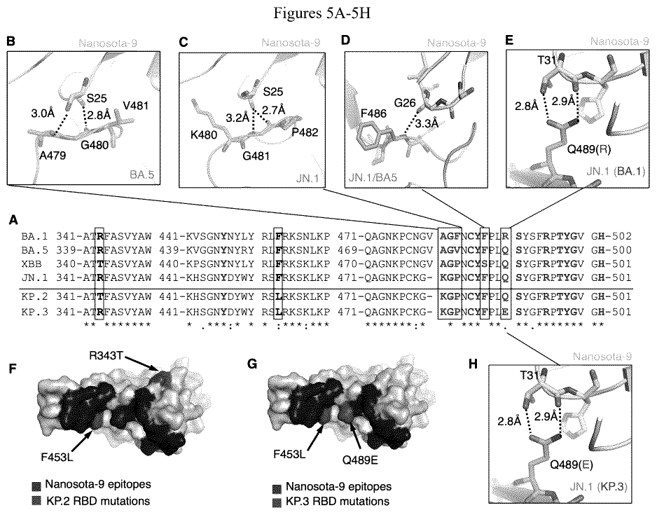

In certain embodiments, the binder protein is capable of binding to a RBD region or residues that is conserved across two or more SARS-CoV-2 Omicron or variants. In certain embodiments, the binder protein is capable of binding to one or more residues (e.g., 2, 3, 4, 5, 6, 7, 8, 9 or more) selected from the group consisting of Y446, G481, C484, Y485, S490, R494, T496, Y497, G498, and H501, wherein the one or more residues are conserved across two or more Omicron or variants (see Example 1, FIG. 5) and the numbering is according to that of Omicron JN.1.

In certain embodiments, the binder protein is capable of binding to JN.1 spike protein RBD at a first interface comprising one or more residues (e.g., 2, 3, 4 or more residues) selected from the group consisting of Y446, P482, Y485, Q489, and R494. In certain embodiments, the one or more residues (e.g., 2, 3 or 4 residues), selected from the group consisting of Y446, P482, Y485, Q489, and R494, are conserved across two or more Omicron or variants and the numbering is according to that of Omicron JN.1. For example, in certain embodiments, Y446, Y485, and R494, are conserved across two or more Omicron or variants

In certain embodiments, the binder protein is capable of binding to JN.1 spike protein RBD at a second interface comprising one or more residues (e.g., 2, 3, 4 or 5 residues) selected from the group consisting of K375, F372, S405, N402, and H501. In certain embodiments, the one or more residues (e.g., 2, 3, 4 or 5 residues), selected from the group consisting of K375, F372, S405, N402, and H501, are conserved across two or more Omicron or variants and the numbering is according to that of Omicron JN.1.

In certain embodiments, the binder protein is capable of binding to KP.3 spike protein RBD having E493 and the numbering is according to that of prototypic SARS-CoV-2 spike protein (equivalent numberings are Q489 in JN.1; E485 in KP.3).

The RBD of SARS-CoV-2 spike protein is capable of adopting both a standing up and lying down configuration. These configurations are known in the art and described herein (e.g., see Example 1). In certain embodiments, the binder protein is capable of binding to the standing up RBD. In certain embodiments, the binder protein is capable of binding to the lying down RBD. In certain embodiments, the binder protein is capable of binding to both the standing up RBD and the lying down RBD.

In certain embodiments, the binder proteins are capable of binding to the trimeric spike protein subunits, for example, three binder protein binds three spike protein subunits. In certain embodiments, two binder proteins crosslink two RBDs in a 2:2 binding mode, wherein one binder protein binds a standing-up RBD, another binder protein binds a lying down RBD, and the two binder proteins also interact with each other at a binder-intermolecular interface.

In certain embodiments, the binder protein is capable of binding to the RBD (e.g., lying down RBD) in a region (e.g., a cryptic region in the trimeric spike) that is inaccessible to or that cannot accommodate a full-size antibody, or an antigen binding fragment thereof having both a heavy chain and light chain (e.g., a scFv, a Fab, or a full-size IgG antibody).

In certain embodiments, a binder protein described herein binds a SARS-CoV-2 spike protein (e.g., Omicron or variant spike protein such as spike ectodomain) with a K_{d} value of about 0.01, 0.02, 0.03, 0.04, 0.05, 0.06, 0.07, 0.08, 0.09, 0.1, 0.5, 1, 5, 10, 15, 20, 25, 30, 40, 50, 60, 70, 80, 90, 100, 200, 300, 400, 500, 600, 700, 800, or 900 nM. In certain embodiments, a binder protein described herein binds a SARS-CoV-2 spike protein (e.g., Omicron or variant spike protein such as ectodomain) with a K_{d} value of about 0.02 to 100 nM, 0.03 to 50 nM, 0.05 to 40 nM, 0.06 to 30 nM, 10 to 100 nM, 20 to 80 nM, or 0.05 to 0.1 nM.

### Tags

In certain embodiments, a binder protein described herein is operably linked to at least one detectable agent (e.g., a polypeptide tag such as His6 tag (SEQ ID NO: 8)). In certain embodiments, an isolated anti-SARS-CoV-2 binder protein as described herein is operably linked to at least one detectable agent. The location of the detectable agent is not critical, provided that it does not interfere with the function of the binder protein. In certain embodiments, the detectable agent is operably linked to the N-terminus of the binder protein. In certain embodiments, the detectable agent is operably linked to the C-terminus of the binder protein.

In certain embodiments, the at least one detectable agent is a tag, such as an affinity tag or an epitope tag. For example, such a tag may be useful for detecting, isolating and/or purifying the binder protein. In certain embodiments, the tag is a polypeptide tag. Polypeptide tags are known in the art and include, but are not limited to, e.g., a His tag, Myc tag, HA tag or an Fc tag. In certain embodiments, the at least one detectable agent is an Fc tag (e.g., an IgG1, IgG2, IgG3, or IgG4 Fc). For example, as described below, a sdAb may be operably linked to an Fc domain amino acid sequence, to produce a sdAb-Fc fusion protein. In certain embodiments, the at least one detectable agent is a His tag (e.g., His6 tag (SEQ ID NO: 8)). In certain embodiments, the at least one detectable agent is a HA tag. In certain embodiments, multiple tags may be operably linked in tandem either directly or via a linker group. In certain embodiments, the detectable agent(s) comprise a HA tag and/or a His tag (e.g., His6 tag (SEQ ID NO: 8)).

In certain embodiments, the binder protein is directly linked to the detectable agent, such as a polypeptide tag (e.g., through a peptide bond).

In certain other embodiments, the binder protein is linked to the detectable agent, such as a polypeptide tag, via one or more optional linker group(s). The nature of the linker group is not critical, provided that the linker group does not interfere with the function of the binder protein or the detectable agent. In certain embodiments, the linker group is an amino acid sequence (e.g., a sequence described herein). In certain embodiments, the linker group is an amino acid sequence that is at least 1, 2, 3, 4, 5, 6, 7, 8, 9, 10 or more amino acids in length. In certain embodiments, the linker group is an amino acid sequence about 1 to about 25 amino acids in length, or about 1 to about 20 amino acids in length, or about 1 to about 15 amino acids in length, or about 1 to about 10 amino acids in length (e.g., 1, 2, 3, 4, 5, 6, 7, 8, 9 or 10 amino acids in length). In certain embodiments, the linker group is glycine rich linker (e.g., having about more than 60% of the amino acid residues in the linker group is glycine). In certain embodiments, the linker group is glycine-serine linker (e.g., GS, GGS, or GGSGGS (SEQ ID NO: 30)).

Accordingly, in certain embodiments, an anti-SARS-CoV-2 binder protein described herein operably linked to a polypeptide tag, comprises an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to:

(SEQ ID NO: 5)
QVQLQESGGGLVQPGGSLRLSCTASGIALHTHATGWFRQAPGKEREGVS
CISSGDGTTYYEDSVEGRFTISRDNAKNTVYLQMNSLKLEDTAVYYCAA
DPGAVCHSGSYYYTDDDFYYRGQGTQVTVSSGGQHHHHHHGAYPYDVPD
YAS.

In some embodiments, an anti-SARS-CoV-2 binder protein comprises an amino acid sequence of SEQ ID NO:5. In some embodiments, an anti-SARS-CoV-2 binder protein consists of the amino acid sequence of SEQ ID NO:5.

In certain embodiments, a detectable agent is a small molecule with molecular weight no greater than 1000 g/mol (e.g., biotin or fluorophore). In certain embodiments, a detectable agent is a nanoparticle (e.g., gold nanoparticle or magnetic particle). In certain embodiments, a detectable agent is suitable for a lateral flow assay. In certain embodiments, a detectable agent (e.g., tag) is an enzyme (e.g., horseradish peroxidase (HRP) or an enzyme that is suitable for a chemiluminescent assay, or a colorimetric assay such as ELISA). In certain embodiments, a detectable agent is suitable for a colorimetric, chemiluminescent, or fluorescent assay. In certain embodiments, a detectable agent is suitable for an ELISA, chemiluminescence assay, or flow cytometry or fluorescent imaging assay.

In some embodiments, the binder protein is encoded by a polynucleotide comprising a nucleic acid sequence that has at least about 80% (e.g., at least about 80%, 85%, 90%, 91%, 92%, 93%, 94%, 95%, 96%, 97%, 98%, 99% or 100%) sequence identity to any one of SEQ ID NOs: 10, 11, 22, 23, or 24.

In certain embodiments, a sdAb as described herein is a recombinant sdAb. In certain embodiments, a sdAb as described herein is a chimeric sdAb. In certain embodiments, a sdAb as described herein is humanized.

In certain embodiments, a sdAb of the invention is a monoclonal sdAb. In some embodiments, the monoclonal sdAb recognizes an epitope within SARS-CoV-2 spike protein.

In certain embodiments, an isolated anti-SARS-CoV-2 binder protein described herein is an inhibitor of SARS-CoV-2.

The term “inhibitor of SARS-CoV-2” as used herein refers to a binder protein that is capable of inhibiting the function of SARS-CoV-2 (e.g., inhibits binding to ACE2). For example, in certain embodiments, a binder protein as described herein detectably inhibits the biological activity of SARS-CoV-2 (e.g., Omicron or variants) as measured, e.g., using an assay described herein. In certain embodiments, the binder protein inhibits the biological activity of SARS-CoV-2 by at least about 5%, at least about 10%, at least about 20%, at least about 30%, at least about 40%, at least about 50%, at least about 60%, at least about 70%, at least about 80%, or at least about 90%.

### Certain Monospecific or Bispecific Binder Proteins of the Invention

As described herein, a binder protein may comprise a sdAb of the invention and may optionally be linked to one or more additional polypeptides. For example, in certain embodiments, an isolated anti-SARS-CoV-2 binder protein described herein is further linked to one or more antibody domain sequences (e.g., heavy or light chain domain sequences, such as variable or constant domain sequences). Accordingly, certain embodiments provide a protein comprising a sdAb of the invention operably linked to one or more antibody domain sequences. Such protein molecules comprising a sdAb of the invention and one or more additional antibody domains may be referenced herein as an antibody or antibody fragment. Additionally, such molecules may be further modified as described herein (e.g., humanized or to alter its affinity, etc.). In certain embodiments, the one or more antibody domain sequences are derived from an antibody class or isotype as defined herein (e.g., IgG (IgG1, IgG2, IgG3, IgG4), IgM, IgA (IgA1 and IgA2), IgD, and IgE).

In certain embodiments, the sdAb is not linked to a light chain domain. In certain embodiments, the sdAb is not linked to a constant domain region. In certain embodiments, the sdAb is not linked to a CH1 region.

In certain embodiments, an isolated anti-SARS-CoV-2 sdAb described herein is linked (e.g., through a linker or a direct bond, such as a peptide bond) to at least one heavy chain constant region (e.g., 1, 2, or 3). In certain embodiments, the sdAb is linked to two heavy chain constant regions (e.g., a CH2 and CH3 region). In certain embodiments, the sdAb is operably linked to an Fc domain amino acid sequence (e.g., an IgG Fc domain such as IgG1, IgG2, IgG3, or IgG4 Fc domain), to produce a sdAb-Fc fusion protein.

Thus, certain embodiments of the invention provide a sdAb-Fc-fusion protein comprising a sdAb of the invention operably linked to a Fc domain amino acid sequence. In certain embodiments, the sdAb and Fc domain amino acid sequence are directly linked, e.g., through a peptide bond. In certain embodiments, the sdAb and Fc domain amino acid sequence are linked through an amino acid linking group. In certain embodiments, the Fc domain amino acid sequence is an IgG1 Fc domain amino acid sequence. In certain embodiments, the Fc domain amino acid sequence has at least about has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:9, 20, or 21. In certain embodiments, the Fc domain amino acid sequence comprises SEQ ID NO:9, 20, or 21. In certain embodiments, the Fc domain amino acid sequence consists of SEQ ID NO:9, 20, or 21.

In certain embodiments, the sdAb-Fc fusion protein comprises an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to a sdAb-Fc sequence described in Table A.

In certain embodiments, the sdAb-Fc fusion protein comprises an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to:

(SEQ ID NO: 6)
QVQLQESGGGLVQPGGSLRLSCTASGIALHTHATGWFRQAPGKEREGVSCISSGDGTTYYEDSV
EGRFTISRDNAKNTVYLQMNSLKLEDTAVYYCAADPGAVCHSGSYYYTDDDFYYRGQGTQVTVS
SEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK;
(SEQ ID NO: 16)
QVQLQESGGGLVQPGGSLRLSCTASGIALHSKATGWFRQAPGKEREGVSCISSGDGTTYYEDSV
EGRFTISRDNAKNTVYLQMNSLKLEDTAVYYCAADPSGVCHSGSYYYTDDDFYYRGQGTQVTVS
SEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK;
(SEQ ID NO: 27)
QVQLQESGGGLVQPGGSLRLSCTASGIALHTHATGWFRQAPGKEREGVSCISSGDGTTYYEDSV
EGRFTISRDNAKNTVYLQMNSLKLEDTAVYYCAADPGAVCHSGSYYYTDDDFYYRGQGTQVTVS
SEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQ
PREPQVYTLPPSREEMTKNQVSLYCLVKGFYPSDIAVEWESNGQPENNYKTTPPVLDSDGSFFL
YSKLTVDKSRWQQGNVFSCSVMHEALHNHYTQKSLSLSPGK;
(SEQ ID NO: 28)
QVQLQESGGGLVQPGGSLRLSCTASGIALHSKATGWFRQAPGKEREGVSCISSGDGTTYYEDSV
EGRFTISRDNAKNTVYLQMNSLKLEDTAVYYCAADPSGVCHSGSYYYTDDDFYYRGQGTQVTVS
SEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFL
TSKLTVDKSRWQQGNVFSCSVMHEALHNHYTQKSLSLSPGK;
or
(SEQ ID NO: 29)
QVQLQESGGGLVQPGGSLRLSCTASGIALHHLATGWFRQAPGKEREGVSCISSGDGTTYYEDSV
EGRFTISRDNAKNTVYLQMNSLKLEDTAVYYCAADPVCHSGSYYYTDDDFYYRGQGTQVTVSSE
PKSCDKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK.

In some embodiments, the sdAb-Fc fusion protein comprises the amino acid sequence of SEQ ID NO:6, 16, 27, 28, or 29. In some embodiments, the sdAb-Fc fusion protein consists of the amino acid sequence of SEQ ID NO:6, 16, 27, 28, or 29.

Certain embodiments of the invention also provide multivalent sdAbs (e.g., bivalent, trivalent, tetravalent, pentavalent or higher valence multivalent sdAbs). Thus, certain embodiments of the invention provide a binder protein comprising two or more independently selected sdAbs as described herein, wherein the sdAbs are operably linked to each other (e.g., to form a dimer, trimer, tetramer, pentamer or higher valence multimer sdAb). In certain embodiments, a multivalent sdAb or binding protein as described herein is a homo-multimer (e.g., dimer, trimer, tetramer or pentamer). In certain embodiments, a multivalent sdAb or binder protein as described herein is a hetero-multimer (e.g., dimer, trimer, tetramer or pentamer).

In certain embodiments, the two or more sdAbs are operably linked via a linker group (e.g., a peptide linker group), disulfide bond(s) and/or by non-covalent interactions. In certain embodiments, the two or more sdAbs are operably linked via oligomerization of tag polypeptides (e.g., multimerization tags, such as a dimerization tags, trimerization tags, tetramerization tags, etc.).

In certain embodiments, the two or more sdAbs are operably linked via a linker group. The nature of the linker group is not critical, provided that the linker group does not interfere with the function of the sdAbs. In certain embodiments, the linker group is a peptide linker group. In certain embodiments, the peptide linker is a glycine-serine rich linker.

In certain embodiments, two independently selected sdAbs are linked via a linker group (e.g., a peptide linker group) to form dimeric sdAb. In certain embodiments, three independently selected sdAbs are linked via two linker groups (e.g., two peptide linker groups) to form a trimeric sdAb. In certain embodiments, four independently selected sdAbs are linked via three linker groups (e.g., three peptide linker groups) to form a tetrameric sdAb. In certain embodiments, five independently selected sdAbs are linked via four linker groups (e.g., four peptide linker groups) to form a pentameric sdAb.

In certain embodiments, the two or more sdAbs are operably linked via oligomerization of tag polypeptides. For example, a sdAb as described herein may be operably linked to a tag polypeptide to form a sdAb-tag fusion protein, wherein the tag polypeptide is capable of oligomerizing. Accordingly, two or more sdAb-tag fusion proteins may be operably linked to form a dimer, trimer, tetramer, pentamer or a higher valence multimer via polypeptide tag-mediated oligomerization.

In certain embodiments, the sdAb and tag polypeptide are linked through a peptide linker to form the sdAb-tag fusion protein. In certain embodiments, a sdAb and tag polypeptide are directly linked without an intervening peptide linker to form the sdAb-tag fusion protein.

In certain embodiments, the tag polypeptide is a human Fc sequence, a human collagen XVIII trimerization domain or a coiled-coil peptide derived from human cartilage oligomeric matrix protein COMP48, which is capable of forming a multimer, such as a pentamer.

In certain embodiments, the tag polypeptide is a human Fc sequence. Accordingly, certain embodiments of the invention provide a binding protein comprising: two independently selected sdAb-Fc fusion proteins as described herein, wherein the two Fc polypeptides are linked to form a dimer (e.g., linked by a covalent bond, such as a disulfide bond, or by non-covalent interactions such as electrostatic interactions, hydrogen bonding, etc.).

In certain embodiments, the two sdAb-Fc fusion proteins are the same. In certain embodiments, the two sdAb-Fc fusion proteins are different. In certain embodiments, sdAb-Fc fusion proteins as described herein can form homo-dimers. In certain embodiments, sdAb-Fc fusion proteins as described herein can form hetero-dimers. In certain embodiments, sdAb-Fc fusion proteins as described herein can form bispecific hetero-dimers having binding affinities for different SARS-Cov-2 protein(s) and/or epitopes.

In certain embodiments, the binding protein is a monospecific binder.

In certain embodiments, the two sdAb-Fc fusion proteins are the same. In certain embodiments, sdAb-Fc fusion proteins as described herein can form homo-dimers. In certain embodiments, the sdAb-Fc fusion protein comprises an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:6, 16, or 29. In certain embodiments, the sdAb-Fc fusion protein comprises an amino acid sequence of SEQ ID NO:6, 16, or 29.

In certain embodiments, the binding protein is a bispecific binder.

In certain embodiments, the two sdAb-Fc fusion proteins are different. In certain embodiments, sdAb-Fc fusion proteins as described herein can form hetero-dimers.

In certain embodiments, a bispecific binder protein comprises a first sdAb domain (e.g., Nanosota-9A VHH sequence) and a second sdAb domain (e.g., Nanosota-9B VHH sequence, or Nanosota-9B-2 VHH sequence).

In certain embodiments,
the first sdAb domain comprises
   (a) a CDR1 comprising an amino acid sequence of SEQ ID NO:2;
   (b) a CDR2 comprising an amino acid sequence of SEQ ID NO:3; and
   (c) a CDR3 comprising an amino acid sequence of SEQ ID NO:4; and
the second sdAb domain comprises
   (a) a CDR1 comprising an amino acid sequence of SEQ ID NO:13;
   (b) a CDR2 comprising an amino acid sequence of SEQ ID NO:3; and
   (c) a CDR3 comprising an amino acid sequence of SEQ ID NO: 14.

In certain embodiments, a bispecific binder protein comprises:
a first sdAb domain (e.g., Nanosota-9A VHH) comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:1; and
a second sdAb domain (e.g., Nanosota-9B VHH) comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 12.

In certain embodiments, a bispecific binder comprises Fc domain amino acid sequences comprising mutation(s) that promote heterodimerization. Technologies, such as “Knobs into Holes”, for making bispecific binder proteins are known in the art and described herein. For example, a bispecific binder protein described herein may comprise a first sdAb-Fc fusion protein comprising a mutation (e.g., T366Y) in the Fc tag, and a second sdAb-Fc fusion protein comprising another mutation (e.g., Y407T) in the Fc tag.

In certain embodiments, a bispecific binder protein comprises:
a first sdAb-Fc fusion protein comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:27; and
a second sdAb-Fc fusion protein comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:28.

In certain embodiments,
the first sdAb domain comprises
   (a) a CDR1 comprising an amino acid sequence of SEQ ID NO:2;
   (b) a CDR2 comprising an amino acid sequence of SEQ ID NO:3; and
   (c) a CDR3 comprising an amino acid sequence of SEQ ID NO:4; and
the second sdAb domain comprises
   (a) a CDR1 comprising an amino acid sequence of SEQ ID NO:18;
   (b) a CDR2 comprising an amino acid sequence of SEQ ID NO:3; and
   (c) a CDR3 comprising an amino acid sequence of SEQ ID NO:19.

In certain embodiments, a bispecific binder protein comprises:
a first sdAb domain (e.g., Nanosota-9A VHH) comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:1; and
a second sdAb domain (e.g., Nanosota-9B-2 VHH) comprising an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:17.

In certain other embodiments, a single sdAb of the invention is operably linked to an Fc dimer.

Certain embodiments of the invention also provide a protein molecule comprising a sdAb as described herein. The terms “protein”, “protein molecule”, and “polypeptide” are used interchangeably herein. In certain embodiments, the term “protein” or “protein molecule” may refer to a single polypeptide or may refer to two or more polypeptides, wherein the two or more polypeptides may be linked by a covalent (e.g., disulfide bridge) or non-covalent interactions.

As used herein, the term “antibody” includes a single-chain variable fragment (scFv), a dimer of a sdAb, a sdAb-Fc fusion protein or dimer thereof, humanized, fully human or chimeric antibodies, single-chain antibodies, diabodies, and antigen-binding fragments of antibodies that do not contain the Fc region (e.g., Fab fragments). In certain embodiments, the antibody is a camelid antibody, human antibody or a humanized antibody. A “humanized” antibody contains only the three CDRs (complementarity determining regions) and sometimes a few carefully selected “framework” residues (the non-CDR portions of the variable regions) from each donor antibody variable region recombinantly linked onto the corresponding frameworks and constant regions of a human antibody sequence.

As used herein, the term “monoclonal sdAb” or “monoclonal antibody” refers to a sdAb/antibody obtained from a group of substantially homogeneous sdAbs/antibodies, that is, a sdAb/antibody group wherein the sdAbs/antibodies constituting the group are homogeneous except for naturally occurring mutants that exist in a small amount. Monoclonal sdAbs/antibodies are highly specific and interact with a single antigenic site. Furthermore, each monoclonal sdAb/antibody targets a single antigenic determinant (epitope) on an antigen, as compared to common polyclonal sdAb/antibody preparations that typically contain various sdAbs/antibodies against diverse antigenic determinants. In addition to their specificity, monoclonal sdAbs/antibodies are advantageous in that they are typically produced from monoclonal cell cultures not contaminated with other immunoglobulins.

The adjective “monoclonal” indicates a characteristic of antibodies and sdAbs obtained from a substantially homogeneous group of antibodies/sdAbs, and does not specify antibodies/sdAbs produced by a particular method. For example, a monoclonal sdAb to be used in the present invention can be produced by, for example, hybridoma methods (Kohler and Milstein, Nature 256:495, 1975) or recombination methods (U.S. Pat. No. 4,816,567). The monoclonal sdAbs used in the present invention can be also isolated from a phage sdAb library (Clackson et al., Nature 352:624-628, 1991; Marks et al., J. Mol. Biol. 222:581-597, 1991). The monoclonal sdAbs of the present invention may be linked to other antibody domain sequences. Therefore, the resulting polypeptides/protein molecules may be “chimeric” immunoglobulins, wherein a part of the polypeptide is derived from a specific species or a specific antibody class or subclass, and the remaining portion is derived from another species, or another antibody class or subclass. Furthermore, mutant sdAbs, as well as mutant polypeptides/protein molecules comprising a sdAb of the invention, are also comprised in the present invention (U.S. Pat. No. 4,816,567; Morrison et al., Proc. Natl. Acad. Sci. USA 81:6851-6855, 1984).

As used herein, the term “mutant sdAb” or “mutant antibody” refers to a sdAb/antibody comprising a variant amino acid sequence in which one or more amino acid residues have been altered. For example, the variable region of a sdAb/antibody can be modified to improve its biological properties, such as antigen binding. Such modifications can be achieved by site-directed mutagenesis (see Kunkel, Proc. Natl. Acad. Sci. USA 82:488 (1985)), PCR-based mutagenesis, cassette mutagenesis, and the like. Such mutants comprise an amino acid sequence which is at least 70% identical to the amino acid sequence of the heavy chain variable region of the sdAb, more specifically at least 75%, even more specifically at least 80%, still more specifically at least 85%, yet more specifically at least 90%, and most specifically at least 95% identical. Such mutants also comprise an amino acid sequence which is at least 70% identical to the amino acid sequence of a heavy or light chain variable region of the antibody, more specifically at least 75%, even more specifically at least 80%, still more specifically at least 85%, yet more specifically at least 90%, and most specifically at least 95% identical. As used herein, the term “sequence identity” is defined as the percentage of residues identical to those in the sdAb's/antibody's original amino acid sequence, determined after the sequences are aligned and gaps are appropriately introduced to maximize the sequence identity as necessary.

Specifically, the identity of one nucleotide sequence or amino acid sequence to another can be determined using the algorithm BLAST, by Karlin and Altschul (Proc. Natl. Acad. Sci. USA, 90:5873-5877, 1993). Programs such as BLASTN and BLASTX were developed based on this algorithm (Altschul et al., J. Mol. Biol. 215:403-410, 1990). To analyze nucleotide sequences according to BLASTN based on BLAST, the parameters are set, for example, as score=100 and wordlength=12. On the other hand, parameters used for the analysis of amino acid sequences by BLASTX based on BLAST include, for example, score=50 and wordlength=3. Default parameters for each program are used when using the BLAST and Gapped BLAST programs. Specific techniques for such analyses are known in the art (see the website of the National Center for Biotechnology Information (NCBI), Basic Local Alignment Search Tool (BLAST); www.ncbi.nlm.nih.gov).

Polyclonal and monoclonal sdAbs/antibodies can be prepared by methods known to those skilled in the art.

In another embodiment, antibodies or antibody fragments (e.g., sdAbs) can be isolated from an antibody/sdAb phage library, produced by using the technique reported by McCafferty et al. (Nature 348:552-554 (1990)). Clackson et al. (Nature 352:624-628 (1991)), Marks et al. (J. Mol. Biol. 222:581-597 (1991)) and Muyldermans et al. (Annual Review of Biochemistry Volume 82, pp 775-797 (2013)) reported on the respective isolation of mouse, camelid and human antibodies from phage libraries. There are also reports that describe the production of high affinity (nM range) human antibodies based on chain shuffling (Marks et al., Bio/Technology 10:779-783 (1992)), and combinatorial infection and in vivo recombination, which are methods for constructing large-scale phage libraries (Waterhouse et al., Nucleic Acids Res. 21:2265-2266 (1993)). These technologies can also be used to isolate monoclonal sdAbs/antibodies, instead of using conventional hybridoma technology for monoclonal sdAb/antibody production.

SdAbs/antibodies to be used in the present invention can be purified by a method appropriately selected from known methods, such as the protein A-Sepharose method, hydroxyapatite chromatography, salting-out method with sulfate, ion exchange chromatography, and affinity chromatography, or by the combined use of the same.

The present invention may use recombinant sdAbs/antibodies, produced by gene engineering. The genes encoding the sdAbs/antibodies obtained by a method described above are isolated from B cells or hybridomas. The genes are inserted into an appropriate vector, and then introduced into a host (see, e.g., Carl, A. K. Borrebaeck, James, W. Larrick, Therapeutic Monoclonal Antibodies, Published in the United Kingdom by Macmillan Publishers Ltd, 1990). The present invention provides the nucleic acids encoding the sdAbs/antibodies of the present invention, and vectors comprising these nucleic acids. Specifically, using a reverse transcriptase, cDNAs encoding the variable region(s) (V region) of the sdAbs/antibodies are synthesized from the mRNAs of B cells or hybridomas. After obtaining the DNAs encoding the variable region(s) of interest, they are optionally ligated with DNAs encoding desired constant regions (C regions), and the resulting DNA constructs are inserted into expression vectors. Alternatively, the DNAs encoding the variable region(s) may be inserted into expression vectors comprising the DNAs of the C regions. These are inserted into expression vectors so that the genes are expressed under the regulation of an expression regulatory region, for example, an enhancer and promoter. Then, host cells are transformed with the expression vectors to express the sdAbs/antibodies. The present invention provides cells expressing sdAbs/antibodies of the present invention. The cells expressing sdAbs/antibodies of the present invention include cells and hybridomas transformed with a gene of such a sdAb/antibody.

The sdAbs/antibodies of the present invention also include sdAbs/antibodies which comprise complementarity-determining regions (CDRs), or regions functionally equivalent to CDRs. The term “functionally equivalent” refers to comprising amino acid sequences similar to the amino acid sequences of CDRs of any of the monoclonal sdAbs isolated in the Examples. The term “CDR” refers to a region in a sdAb/antibody variable region (also called “V region”), and determines the specificity of antigen binding. The H chain and L chain (if present) each have three CDRs, designated from the N terminus as CDR1, CDR2, and CDR3. There are four regions flanking these CDRs: these regions are referred to as “framework,” and their amino acid sequences are highly conserved. The CDRs can be transplanted into other sdAbs/antibodies, and thus a recombinant antibody can be prepared by combining CDRs with the framework of a desired sdAb/antibody. One or more amino acids of a CDR can be modified without losing the ability to bind to its antigen. For example, one or more amino acids in a CDR can be substituted, deleted, and/or added.

In certain embodiments, an amino acid residue is mutated into one that allows the properties of the amino acid side-chain to be conserved. Examples of the properties of amino acid side chains comprise: hydrophobic amino acids (A, I, L, M, F, P, W, Y, V), hydrophilic amino acids (R, D, N, C, E, Q, G, H, K, S, T), and amino acids comprising the following side chains: aliphatic side-chains (G, A, V, L, I, P); hydroxyl group-containing side-chains (S, T, Y); sulfur atom-containing side-chains (C, M); carboxylic acid- and amide-containing side-chains (D, N, E, Q); base-containing side-chains (R, K, H); and aromatic-containing side-chains (H, F, Y, W). The letters within parenthesis indicate the one-letter amino acid codes. Amino acid substitutions within each group are called conservative substitutions. It is well known that a polypeptide comprising a modified amino acid sequence in which one or more amino acid residues is deleted, added, and/or substituted can retain the original biological activity (Mark D. F. et al., Proc. Natl. Acad. Sci. U.S.A. 81:5662-5666 (1984); Zoller M. J. and Smith M., Nucleic Acids Res. 10:6487-6500 (1982); Wang A. et al., Science 224:1431-1433; Dalbadie-McFarland G. et al., Proc. Natl. Acad. Sci. U.S.A. 79:6409-6413 (1982)). The number of mutated amino acids is not limited, but in general, the number falls within 40% of amino acids of each CDR, and specifically within 35%, and still more specifically within 30% (e.g., within 25%). The identity of amino acid sequences can be determined as described herein.

In the present invention, recombinant sdAbs/antibodies artificially modified to reduce heterologous antigenicity against humans can be used. Examples include chimeric sdAbs/antibodies and humanized sdAbs/antibodies. These modified sdAbs/antibodies can be produced using known methods. A chimeric antibody includes an antibody comprising variable and constant regions of species that are different to each other, for example, an antibody comprising the antibody heavy chain and light chain variable regions of a nonhuman mammal such as a mouse, and the antibody heavy chain and light chain constant regions of a human. Additionally, a chimeric antibody or polypeptide may be produced by combining a sdAb of the invention with constant regions that are of different species to each other. Such an antibody (e.g., a camelid-human chimeric antibody) can be obtained by (1) ligating a DNA from the different regions; (2) incorporating this into an expression vector; and (3) introducing the vector into a host for production of the antibody.

A humanized sdAb/antibody, which is also called a reshaped human sdAb/antibody, may be obtained by replacing a CDR of a human antibody with an H or L chain CDR of a sdAb/antibody of a nonhuman mammal such as a mouse or camelid. Conventional genetic recombination techniques for the preparation of such antibodies are known (see, for example, Jones et al., Nature 321:522-525 (1986); Reichmann et al., Nature 332:323-329 (1988); Presta Curr. Op. Struct. Biol. 2:593-596 (1992)). Specifically, a DNA sequence designed to ligate a CDR of a mouse/camelid antibody with the framework regions (FRs) of a human antibody is synthesized by PCR, using several oligonucleotides constructed to comprise overlapping portions at their ends. A humanized antibody can be obtained by (1) ligating the resulting DNA to a DNA that encodes a human antibody constant region; (2) incorporating this into an expression vector; and (3) transfecting the vector into a host to produce the antibody (see, European Patent Application No. EP 239,400, and International Patent Application No. WO 96/02576). Human antibody FRs that are ligated via the CDR are selected where the CDR forms a favorable antigen-binding site. The humanized antibody may comprise additional amino acid residue(s) that are not included in the CDRs introduced into the recipient antibody, nor in the framework sequences. Such amino acid residues are usually introduced to more accurately optimize the antibody's ability to recognize and bind to an antigen. For example, as necessary, amino acids in the framework region of a variable region may be substituted such that the CDR of a reshaped human antibody forms an appropriate antigen-binding site (Sato, K. et al., Cancer Res. (1993) 53, 851-856).

Isotypes of sdAb-fusion proteins or antibodies comprising a sdAb of the present invention, or antibody fragments thereof, are not limited. The isotypes include, for example, IgG (IgG1, IgG2, IgG3, and IgG4), IgM, IgA (IgAQ1 and IgA2), IgD, and IgE.

As described herein, a sdAb of the present invention may be operably linked to one or more antibody domain sequences (e.g., a sdAb-Fc fusion protein). Therefore, such polypeptides/protein molecules comprising a sdAb of the invention and one or more additional antibody domains may be referenced herein as an antibody or antibody fragment. The term “antibody fragment” refers to a portion of a full-length antibody, and generally to a fragment comprising an antigen-binding domain or a variable region. Such antibody fragments include, for example, single domain antibody (sdAb), Fab, F(ab′)₂, Fv, single-chain Fv (scFv) which comprises a heavy chain Fv and a light chain Fv coupled together with an appropriate linker, diabody (diabodies), linear antibodies, and multispecific antibodies prepared from antibody fragments. Previously, antibody fragments were produced by digesting natural antibodies with a protease; currently, methods for expressing them as recombinant antibodies using genetic engineering techniques are also known (see Morimoto et al., Journal of Biochemical and Biophysical Methods 24:107-117 (1992); Brennan et al., Science 229:81 (1985); Co, M. S. et al., J. Immunol., 1994, 152, 2968-2976; Better, M. & Horwitz, A. H., Methods in Enzymology, 1989, 178, 476-496, Academic Press, Inc.; Plueckthun, A. & Skerra, A., Methods in Enzymology, 1989, 178, 476-496, Academic Press, Inc.; Lamoyi, E., Methods in Enzymology, 1989, 121, 663-669; Bird, R. E. et al., TIBTECH, 1991, 9, 132-137).

The sdAbs/antibodies can be purified to homogeneity. The sdAbs/antibodies can be isolated and purified by a method routinely used to isolate and purify proteins. The sdAbs/antibodies can be isolated and purified by the combined use of one or more methods appropriately selected from column chromatography, filtration, ultrafiltration, salting out, dialysis, preparative polyacrylamide gel electrophoresis, and isoelectro-focusing, for example (Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory, 1988). Such methods are not limited to those listed above. Chromatographic methods include affinity chromatography, ion exchange chromatography, hydrophobic chromatography, gel filtration, reverse-phase chromatography, and adsorption chromatography. These chromatographic methods can be practiced using liquid phase chromatography, such as HPLC and FPLC. Columns to be used in affinity chromatography include protein A columns and protein G columns. For example, protein A columns include Hyper D, POROS, and Sepharose F. F. (Pharmacia). SdAbs/antibodies can also be purified by utilizing antigen binding, using carriers on which antigens have been immobilized.

The sdAbs/antibodies of the present invention can be formulated according to standard methods (see, for example, Remington's Pharmaceutical Science, latest edition, Mark Publishing Company, Easton, U.S.A), and may comprise pharmaceutically acceptable carriers and/or additives. The present invention relates to compositions (including reagents and pharmaceuticals) comprising the sdAbs/antibodies of the invention, and pharmaceutically acceptable carriers and/or additives. Exemplary carriers include surfactants (for example, PEG and Tween), excipients, antioxidants (for example, ascorbic acid), coloring agents, flavoring agents, preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, and corrigents. However, the carriers that may be employed in the present invention are not limited to this list. In fact, other commonly used carriers can be appropriately employed: light anhydrous silicic acid, lactose, crystalline cellulose, mannitol, starch, carmelose calcium, carmelose sodium, hydroxypropylcellulose, hydroxypropylmethyl cellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, inorganic salt, and so on. The composition may also comprise other low-molecular-weight polypeptides, proteins such as serum albumin, gelatin, and immunoglobulin, and amino acids such as glycine, glutamine, asparagine, arginine, and lysine. When the composition is prepared as an aqueous solution for injection, it can comprise an isotonic solution comprising, for example, physiological saline, dextrose, and other adjuvants, including, for example, D-sorbitol, D-mannose, D-mannitol, and sodium chloride, which can also contain an appropriate solubilizing agent, for example, alcohol (for example, ethanol), polyalcohol (for example, propylene glycol and PEG), and non-ionic detergent (polysorbate 80 and HCO-50).

### Nucleic Acids, Expression Cassettes, Vectors and Cells

Certain embodiments of the invention provide an isolated nucleic acid encoding a binder protein as described herein (e.g., a sdAb or sdAb-Fc as described herein). Certain embodiments of the invention provide an isolated nucleic acid comprising a sequence that has at least about 80% (e.g., at least about 80%, 85%, 90%, 91%, 92%, 93%, 94%, 95%, 96%, 97%, 98%, 99% or 100%) sequence identity to SEQ ID NO: 10 or 11. In certain embodiments, the isolated nucleic acid comprises SEQ ID NO: 10 or 11.

In certain embodiments, the nucleic acid further comprises a promoter.

Certain embodiments of the invention provide an expression cassette comprising a nucleic acid as described herein and a promoter.

Certain embodiments of the invention provide a vector (e.g., a phagemid, Adeno-associated viruses (AAV)) comprising a nucleic acid or an expression cassette as described herein.

Certain embodiments of the invention provide a cell comprising a nucleic acid, expression cassette or vector as described herein. In certain embodiments, the cell is a bacterial cell.

In certain embodiments, the cell is a mammalian cell.

In certain embodiments, the cell is a human mammalian cell. In certain embodiments, the cell is a human embryonic kidney (HEK) 293 cell. In certain embodiments, the cell is a 293F cell. In certain embodiments, the cell is a 293T cell. In certain embodiments, the cell is a human embryonic retinal (PER.C6) cell. In certain embodiments, the cell is a HT-1080 cell. In certain embodiments, the cell is a Huh-7 cell.

In certain embodiments, the cell is a non-human mammalian cell. In certain embodiments, the cell is a Monkey kidney epithelial (Vero) cell. In certain embodiments, the cell is a Chinese Hamster Ovary (CHO) cell. In certain embodiments, the cell is a baby hamster kidney (BHK) cell.

In certain embodiments, the cell is a non-mammalian cell. In certain embodiments, the cell is an insect cell. In certain embodiments, the cell is a yeast cell.

### Compositions and Kits

Certain embodiments provide a composition comprising an anti-SARS-CoV-2 binder protein as described herein and a carrier. In certain embodiments, the composition is a pharmaceutical composition comprising a pharmaceutically acceptable carrier.

In certain embodiments, the composition is a liquid composition. In certain embodiments, the composition is a solid composition (e.g., powder or lyophilized formulation). In certain embodiments, the composition is a lyophilized composition that further comprises one or more excipients selected from the group consisting of a cryo-lyoprotectant (e.g., trehalose, sucrose) and a bulking agent (e.g., mannitol, glycine). In certain embodiments, the solid composition may be reconstituted (e.g., with water, saline or Dextrose solution) prior to use.

In certain embodiments, a composition is administered subcutaneously, intradermally, intranasally, intramuscularly, intravenously or intraperitoneally. In certain embodiments, a composition is administered via injection, infusion, or nasal spray.

Certain embodiments also provide a kit comprising an isolated anti-SARS-CoV-2 binder protein as described herein, packaging material, and instructions for administering the binder protein/vector, to a mammal to treat a SARS-CoV-2 infection. In certain embodiments, the kit further comprises at least one additional therapeutic agent. In certain embodiments, the at least one additional therapeutic agent is useful for preventing or treating a viral infection or inflammation. In certain embodiments, the at least one additional therapeutic agent is an antibody or a sdAb.

In certain embodiments, the kit further comprises a syringe (e.g., a pre-filled syringe) or a vial comprising the composition as described herein.

In certain embodiments, the kit further comprises an atomizer nozzle for nasal or pulmonary delivery, wherein the atomizer nozzle is or could be fitted with the syringe or vial to produce a spray or mist.

In certain embodiments, the kit further comprises an inhaler device.

In certain embodiments, the kit further comprises a needle that is or could be fitted with the syringe (e.g., to deliver subcutaneous, intradermal, or intramuscular injection).

### Methods of Use

Certain embodiments provide a method of inhibiting the activity of SARS-CoV-2 (e.g., Omicron or variant thereof), comprising contacting SARS-CoV-2 with an isolated anti-SARS-CoV-2 binder protein as described herein, (e.g., under conditions suitable for binding between the binder protein and SARS-CoV-2, such as between the binder protein and the SARS-CoV-2 RBD). In certain embodiments, binding between SARS-CoV-2 (e.g., Omicron or variant thereof) and ACE2 is inhibited.

In certain embodiments, the SARS-CoV-2 (e.g., Omicron or variant thereof) is contacted in vitro. In certain embodiments, the SARS-CoV-2 (e.g., Omicron or variant thereof) is contacted in vivo.

Methods for measuring the activity of SARS-CoV-2 are known in the art. For example, in certain embodiments, an assay described herein may be used. In certain embodiments, a binder protein of the invention inhibits the activity of SARS-CoV-2 (e.g., its ability to infect cell) by at least about 5%, at least about 10%, at least about 15%, at least about 20%, at least about 25%, at least about 30%, at least about 40%, at least about 50%, at least about 60%, at least about 70%, at least about 80%, at least about 90%, at least about 95%, at least about 99% or at least about 100% as compared to a control.

In certain embodiments, a binder protein of the invention inhibits the activity of a SARS-CoV-2 (e.g., live or pseudovirus of Omicron or variant) with a IC₅₀ potency of about 0.1, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, or 900 ng/mL. In certain embodiments, a binder protein of the invention inhibits the activity of a SARS-CoV-2 (e.g., live or pseudovirus of Omicron or variant) with a IC₅₀ potency of about 0.1 to 300, 1 to 200, 2 to 160, 3 to 150, 4 to 120, 5 to 16, 5 to 20, 10 to 100, or 5 to 100 ng/mL.

Certain embodiments also provide a method for treating or preventing a SARS-CoV-2 (e.g., Omicron or variant thereof) infection in a mammal, comprising administering an effective amount of an isolated anti-SARS-CoV-2 binder protein (e.g., Nanosota-9A or Nanosota-9A-Fc; Nanosota-9B or Nanosota-9B-Fc; or a bispecific binder), or a vector as described herein to the mammal. In certain embodiments, the SARS-CoV-2 is SARS-CoV-2 Omicron or variant thereof as described herein (e.g., BA.5, XBB.1.5, or JN.1, KP.2, or KP.3).

In certain embodiments, a binder protein of the invention reduces the titer of a SARS-CoV-2 (e.g., live virus of Omicron or variant) in the lung by about 5, 10, 50, 100, 500, 1000, 5000, 10000-fold, or more, as compared to a control.

In certain embodiments, the method further comprises administering at least one additional therapeutic agent to the mammal. In certain embodiments, the at least one additional therapeutic agent is useful for treating a viral infection or inflammation. In certain embodiments, the at least one additional therapeutic agent is an antibody or a sdAb.

Certain embodiments provide an isolated anti-SARS-CoV-2 binder protein, or vector as described herein for the prophylactic or therapeutic treatment of a SARS-CoV-2 (e.g., Omicron or variant thereof) infection.

Certain embodiments provide the use of an isolated anti-SARS-CoV-2 binder protein or vector as described herein to prepare a medicament for the treatment of a SARS-CoV-2 (e.g., Omicron or variant thereof) infection in a mammal.

Certain embodiments provide an isolated anti-SARS-CoV-2 binder protein or vector as described herein for use in medical therapy.

### Administration

For in vivo use, a protein molecule as described herein (e.g., a sdAb of the invention, or a polypeptide or protein molecule comprising such a sdAb), or a vector, as described herein is generally incorporated into a pharmaceutical composition prior to administration. Within such compositions, one or more protein molecules or vectors of the invention may be present as active ingredient(s) (i.e., are present at levels sufficient to provide a statistically significant effect on the symptoms of a relevant disease (e.g., a SARS-CoV-2 infection), as measured using a representative assay). A pharmaceutical composition comprises one or more such protein molecules or vectors in combination with any pharmaceutically acceptable carrier(s) known to those skilled in the art to be suitable for the particular mode of administration. In addition, other pharmaceutically active ingredients (including other therapeutic agents) may, but need not, be present within the composition.

In certain embodiments, a binder protein or composition described herein could be administered to an animal (e.g., mammal such as human) in need of before or after virus infection, for example, for prevention or treatment of SARS-CoV-2 infection. In certain embodiments, a binder protein or composition described herein could be administered to an animal about 3, 2, 1 week(s) or 6, 5, 4, 3, 2, 1 day(s) or 20, 15, 10, 5, 1 hour(s) before infection or before potential exposure to SARS-CoV-2. In certain embodiments, a binder protein or composition described herein could be administered to an animal about 1, 2, 3, 4, 5, 6, 7, 8, 9, 10, 11, 12, 13, 14, 15, 16, 17, 18, 19, 20, 21, 22, 23 hour(s), 1, 2, 3, 4, 5, 6 day(s), or 1, 2, 3 week(s) after suspected or confirmed infection.

In certain embodiments, a binder protein (e.g., a sdAb, a sdAb-Fc, or a protein comprising the sdAb) is administered to an animal (e.g., mammal such as human) in need of at a dosage of about 1, 2, 3, 4, 5, 6, 7, 8, 9, 10, 11, 12, 13, 14, 15, 16, 17, 18, 19, 20, 21, 22, 23, 25, 26, 27, 28, 29, or 30 mg/kg. In certain embodiments, a binder protein (e.g., a sdAb, a sdAb-Fc, or a protein comprising the sdAb) is administered to an animal (e.g., mammal such as human) in need of at a dosage range of about 0.1 to 50, 0.5 to 40, 1 to 30, 2 to 25, 3 to 20, 4 to 18, or 5 to 16 mg/kg.

In certain embodiments, a binder protein is administered subcutaneously, intradermally, intranasally, intramuscularly, intravenously or intraperitoneally.

The term “therapeutically effective amount,” in reference to treating a disease state/condition, refers to an amount of a protein molecule or vector either alone or as contained in a pharmaceutical composition that is capable of having any detectable, positive effect on any symptom, aspect, or characteristics of a disease state/condition when administered as a single dose or in multiple doses. Such effect need not be absolute to be beneficial.

The terms “treat” and “treatment” refer to both therapeutic treatment and prophylactic or preventative measures, wherein the object is to prevent or decrease an undesired physiological change or disorder, such as a SARS-CoV-2 infection. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

In certain embodiments, the present protein molecules/vectors may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the protein molecule/vector may be combined with one or more excipients and used in the form of ingestible tablets, buccal tablets, troches, capsules, elixirs, suspensions, syrups, wafers, and the like. Such compositions and preparations should contain at least 0.1% of a protein molecule/vector of the present invention. The percentage of the compositions and preparations may, of course, be varied and may conveniently be between about 2 to about 60% of the weight of a given unit dosage form. The amount of protein molecule/vector in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the protein molecule/vector, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the protein molecule/vector may be incorporated into sustained-release preparations and devices.

The protein molecule or a vector as described herein may also be administered subcutaneously, intradermally, intranasally, intramuscularly, intravenously or intraperitoneally by infusion or injection. The protein molecule or a vector as described herein may also be administered via intranasal and/or pulmonary delivery (e.g., delivered as a spray or mist). Additionally, the protein molecule or vector may be administered by local injection, such as by intrathecal injection, epidural injection or peri-neural injection using a scope. Solutions of the protein molecule or vector may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the protein molecule or vector that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be useful to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the protein molecule or vector in the required amount in the appropriate solvent with various of the other ingredients enumerated above, as required, followed by filter sterilization. In the case of sterile powders for the preparation of sterile injectable solutions, the methods of preparation are vacuum drying and the freeze drying techniques, which yield a powder of the protein molecule or vector plus any additional desired ingredient present in the previously sterile-filtered solutions.

For topical administration, the present protein molecules/vectors may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present protein molecules/vectors can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions that can be used to deliver the protein molecules/vectors of the present invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the protein molecules or vectors of the present invention can be determined by comparing their in vitro activity, and in vivo activity in animal models. Methods for the extrapolation of effective dosages in mice, and other animals, to humans are known to the art; for example, see U.S. Pat. No. 4,938,949.

The amount of a protein molecule or vector of the present invention required for use in treatment will vary with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Protein molecules or vectors of the invention can also be administered in combination with other therapeutic agents and/or treatments, such as other agents or treatments that are useful for the treatment of a SARS-CoV-2 infection. In certain embodiments such an agent is an antibody or a sdAb. Additionally, one or more protein molecules or vectors of the invention, may be administered (e.g., a combination of sdAbs, polypeptides, protein molecules and/or vectors may be administered). Accordingly, one embodiment the invention also provides a composition comprising a protein molecule or vector of the invention, at least one other therapeutic agent, and a pharmaceutically acceptable diluent or carrier. The invention also provides a kit comprising a protein molecule or vector of the invention, at least one other therapeutic agent, packaging material, and instructions for administering a protein molecule or vector of the invention, and the other therapeutic agent or agents to an animal to treat a SARS-CoV-2 infection.

As used herein, the term “therapeutic agent” refers to any agent or material that has a beneficial effect on the mammalian recipient.

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucl. Acids Res., 19:508 (1991); Ohtsuka et al., JBC, 260:2605 (1985); Rossolini et al., Mol. Cell. Probes, 8:91 (1994). A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene.

By “portion” or “fragment,” as it relates to a nucleic acid molecule, sequence or segment of the invention, when it is linked to other sequences for expression, is meant a sequence having at least 80 nucleotides, more specifically at least 150 nucleotides, and still more specifically at least 400 nucleotides. If not employed for expressing, a “portion” or “fragment” means at least 9, specifically 12, more specifically 15, even more specifically at least 20, consecutive nucleotides, e.g., probes and primers (oligonucleotides), corresponding to the nucleotide sequence of the nucleic acid molecules of the invention.

The invention encompasses isolated or substantially purified nucleic acid or protein compositions. In the context of the present invention, an “isolated” or “purified” DNA molecule or an “isolated” or “purified” polypeptide is a DNA molecule or polypeptide that exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, culture medium may represent less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have at least 40, 50, 60, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

A “vector” is defined to include, inter alia, any plasmid, cosmid, viral vector, phage or binary vector in double or single stranded linear or circular form which may or may not be self transmissible or mobilizable, and which can transform prokaryotic or eukaryotic host either by integration into the cellular genome or exist extrachromosomally (e.g., autonomous replicating plasmid with an origin of replication).

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

As used herein, the term “operably linked” refers to a linkage of two elements in a functional relationship. For example, “operably linked” may refer to a linkage of polynucleotide elements or polypeptide elements in a functional relationship. A nucleic acid is “operably linked” when it is placed into a functional relationship with another nucleic acid sequence. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. “Operably-linked” also refers to the association two chemical moieties so that the function of one is affected by the other, e.g., an arrangement of elements wherein the components so described are configured so as to perform their usual function.

“Expression” refers to the transcription and/or translation in a cell of an endogenous gene, transgene, as well as the transcription and stable accumulation of sense (mRNA) or functional RNA. In the case of antisense constructs, expression may refer to the transcription of the antisense DNA only. Expression may also refer to the production of protein.

The following terms are used to describe the sequence relationships between two or more sequences (e.g., nucleic acids, polynucleotides or polypeptides): (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA, gene sequence or peptide sequence, or the complete cDNA, gene sequence or peptide sequence.

(b) As used herein, “comparison window” makes reference to a contiguous and specified segment of a sequence, wherein the sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the sequence a gap penalty is typically introduced and is subtracted from the number of matches.

Computer implementations of these mathematical algorithms can be utilized for comparison of sequences to determine sequence identity. Such implementations include, but are not limited to: CLUSTAL in the PC/Gene program (available from Intelligenetics, Mountain View, California); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wisconsin, USA). Alignments using these programs can be performed using the default parameters. The CLUSTAL program is well described by Higgins et al., Gene, 73:237 (1988); Higgins et al., CABIOS, 5:151 (1989); Corpet et al., Nucl. Acids Res., 16:10881 (1988); Huang et al., CABIOS, 8:155 (1992); and Pearson et al., Meth. Mol. Biol., 24:307 (1994). The ALIGN program is based on the algorithm of Myers and Miller, supra. The BLAST programs of Altschul et al., JMB, 215:403 (1990); Nucl. Acids Res., 25:3389 (1990), are based on the algorithm of Karlin and Altschul supra.

Software for performing BLAST analyses is publicly available through the National Center for Biotechnology Information (available on the world wide web at ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more specifically less than about 0.01, and most specifically less than about 0.001.

To obtain gapped alignments for comparison purposes, Gapped BLAST (in BLAST 2.0) can be utilized as described in Altschul et al., Nucleic Acids Res. 25:3389 (1997). Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the world wide web at ncbi.nlm.nih.gov. Alignment may also be performed manually by visual inspection.

For purposes of the present invention, comparison of sequences for determination of percent sequence identity to another sequence may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

(d) As used herein, “percentage of sequence identity” means the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of sequences means that a polynucleotide comprises a sequence that has at least 70%, 71%, 72%, 73%, 74%, 75%, 76%, 77%, 78%, or 79%, at least 80%, 81%, 82%, 83%, 84%, 85%, 86%, 87%, 88%, or 89%, at least 90%, 91%, 92%, 93%, or 94%, and at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, at least 95%.

(e)(ii) The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to the reference sequence over a specified comparison window. Optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, Proc. Natl. Acad. Sci. USA, 82:488 (1985); Kunkel et al., Meth. Enzymol., 154:367 (1987); U.S. Pat. No. 4,873,192; Walker and Gaastra, Techniques in Mol. Biol. (MacMillan Publishing Co. (1983), and the references cited therein. Guidance as to appropriate amino acid substitutions that do not affect biological activity of the protein of interest may be found in the model of Dayhoff et al., Atlas of Protein Sequence and Structure (Natl. Biomed. Res. Found. 1978). Conservative substitutions, such as exchanging one amino acid with another having similar properties, are preferred.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). In addition, individual substitutions, deletions or additions which alter, add or delete a single amino acid or a small percentage of amino acids in an encoded sequence are also “conservatively modified variations.”

TABLE A
Exemplary binder sequences

| SEQ ID | | |
|---|---|---|
| NO: | Sequences | Comment |
| 1 | QVQLQESGGGLVQPGGSLRLSCTASGIALHTHATGWFRQA | Nanosota-9 VHH |
| | PGKEREGVSCISSGDGTTYYEDSVEGRFTISRDNAKNTVY | sequence (Also |
| | LQMNSLKLEDTAVYYCAADPGAVCHSGSYYYTDDDFYYRG | referred to as |
| | QGTQVTVSS | Nanosota-9A VHH) |
| 2 | TASGIALHTH | Nanosota-9: CDR-H1 |
| 3 | ISSGDGTT | Nanosota-9: CDR-H2 |
| 4 | DPGAVCHSGSYYYTDDDFYY | Nanosota-9: CDR-H3 |
| 5 | QVQLQESGGGLVQPGGSLRLSCTASGIALHTHATGWFRQA | Nanosota-9 VHH |
| | PGKEREGVSCISSGDGTTYYEDSVEGRFTISRDNAKNTVY | sequence with linker |
| | LQMNSLKLEDTAVYYCAADPGAVCHSGSYYYTDDDFYYRG | (bold) and His6 tag |
| | QGTQVTVSSGGQHHHHHHGAYPYDVPDYAS | (SEQ ID NO: 8)/HA |
| | | tag (italics) |
| 6 | QVQLQESGGGLVQPGGSLRLSCTASGIALHTHATGWFRQA | Nanosota-9 VHH |
| | PGKEREGVSCISSGDGTTYYEDSVEGRFTISRDNAKNTVY | sequence with IgG1 |
| | LQMNSLKLEDTAVYYCAADPGAVCHSGSYYYTDDDFYYRG | Fc domain |
| | QGTQVTVSSEPKSCDKTHTCPPCPAPELLGGPSVFLFPPK | sequence (bold) |
| | PKDTLMISRTPEVTCVVVDVSHEDPEVKFNWYVDGVEVHN | |
| | AKTKPREEQYNSTYRVVSVLTVLHQDWLNGKEYKCKVSNK | |
| | ALPAPIEKTISKAKGQPREPQVYTLPPSREEMTKNQVSLT | |
| | CLVKGFYPSDIAVEWESNGQPENNYKTTPPVLDSDGSFFL | |
| | YSKLTVDKSRWQQGNVFSCSVMHEALHNHYTQKSLSLSPG | |
| | K | |
| 10 | CAGGTGCAGCTGCAGGAGTCTGGGGGAGGCTTGGTGCAGC | DNA sequence for |
| | CTGGGGGCTCTCTGAGACTCTCCTGTACAGCCTCTGGAAT | Nanosota-9 VHH |
| | CGCTTTGCATACTCATGCCACAGGCTGGTTCCGCCAGGCC | |
| | CCAGGGAAGGAGCGCGAGGGGGTCTCATGTATTAGTAGTG | |
| | GTGATGGTACCACATACTATGAAGACTCCGTGGAGGGGCG | |
| | ATTCACCATCTCCAGAGACAATGCCAAGAACACGGTGTAT | |
| | CTGCAAATGAACAGCCTGAAACTTGAGGATACGGCCGTTT | |
| | ATTACTGTGCGGCGGATCCGGGGGCGGTATGCCATAGTGG | |
| | CAGTTACTACTATACGGATGACGACTTCTACTACCGGGGC | |
| | CAGGGGACCCAGGTCACCGTCTCCTCA | |
| 11 | CAGGTGCAGCTGCAGGAGTCTGGGGGAGGCTTGGTGCAGC | DNA sequence for |
| | CTGGGGGCTCTCTGAGACTCTCCTGTACAGCCTCTGGAAT | Nanosota-9-Fc |
| | CGCTTTGCATACTCATGCCACAGGCTGGTTCCGCCAGGCC | |
| | CCAGGGAAGGAGCGCGAGGGGGTCTCATGTATTAGTAGTG | |
| | GTGATGGTACCACATACTATGAAGACTCCGTGGAGGGGCG | |
| | ATTCACCATCTCCAGAGACAATGCCAAGAACACGGTGTAT | |
| | CTGCAAATGAACAGCCTGAAACTTGAGGATACGGCCGTTT | |
| | ATTACTGTGCGGCGGATCCGGGGGCGGTATGCCATAGTGG | |
| | CAGTTACTACTATACGGATGACGACTTCTACTACCGGGGC | |
| | CAGGGGACCCAGGTCACCGTCTCCTCAGAGCCCAAATCTT | |
| | GTGACAAAACTCACACATGCCCACCGTGCCCAGCACCTGA | |
| | ACTCCTGGGGGGACCGTCAGTCTTCCTCTTCCCCCCAAAA | |
| | CCCAAGGACACCCTCATGATCTCCCGGACCCCTGAGGTCA | |
| | CATGCGTGGTGGTGGACGTGAGCCACGAAGACCCTGAGGT | |
| | CAAGTTCAACTGGTACGTGGACGGCGTGGAGGTGCATAAT | |
| | GCCAAGACAAAGCCGCGGGAGGAGCAGTACAACAGCACGT | |
| | ACCGTGTGGTCAGCGTCCTCACCGTCCTGCACCAGGACTG | |
| | GCTGAATGGCAAGGAGTACAAGTGCAAGGTCTCCAACAAA | |
| | GCCCTCCCAGCCCCCATCGAGAAAACCATCTCCAAAGCCA | |
| | AAGGGCAGCCCCGAGAACCACAGGTGTACACCCTGCCCCC | |
| | ATCCCGGGAGGAGATGACCAAGAACCAGGTCAGCCTGACC | |
| | TGCCTGGTCAAAGGCTTCTATCCCAGCGACATCGCCGTGG | |
| | AGTGGGAGAGCAATGGGCAGCCGGAGAACAACTACAAGAC | |
| | CACGCCTCCCGTGCTGGACTCCGACGGCTCCTTCTTCCTC | |
| | TACAGCAAGCTCACCGTGGACAAGAGCAGGTGGCAGCAGG | |
| | GGAACGTCTTCTCATGCTCCGTGATGCATGAGGCTCTGCA | |
| | CAACCACTACACGCAGAAGAGCCTCTCCCTGTCTCCGGGT | |
| | AAA | |
| | GGQ | Linker sequence |
| 8 | HHHHHH | His6 tag sequence |
| 9 | EPKSCDKTHTCPPCPAPELLGGPSVFLFPPKPKDTLMISR | IgG1 Fc sequence |
| | TPEVTCVVVDVSHEDPEVKFNWYVDGVEVHNAKTKPREEQ | |
| | YNSTYRVVSVLTVLHQDWLNGKEYKCKVSNKALPAPIEKT | |
| | ISKAKGQPREPQVYTLPPSREEMTKNQVSLTCLVKGFYPS | |
| | DIAVEWESNGQPENNYKTTPPVLDSDGSFFLYSKLTVDKS | |
| | RWQQGNVFSCSVMHEALHNHYTQKSLSLSPGK | |
| 12 | QVQLQESGGGLVQPGGSLRLSCTASGIALHSKATGWFRQA | Nanosota-9B VHH |
| | PGKEREGVSCISSGDGTTYYEDSVEGRFTISRDNAKNTVY | sequence (also |
| | LQMNSLKLEDTAVYYCAADPSGVCHSGSYYYTDDDFYYRG | referred to as |
| | QGTQVTVSS | Nanosota-9B-1 VHH) |
| 13 | TASGIALHSK | Nanosota-9B: CDR-H1 |
| 3 | ISSGDGTT | Nanosota-9B: CDR-H2 |
| 14 | DPSGVCHSGSYYYTDDDFYY | Nanosota-9B: CDR-H3 |
| 15 | QVQLQESGGGLVQPGGSLRLSCTASGIALHSKATGWFRQA | Nanosota-9B VHH |
| | PGKEREGVSCISSGDGTTYYEDSVEGRFTISRDNAKNTVY | sequence with linker |
| | LQMNSLKLEDTAVYYCAADPSGVCHSGSYYYTDDDFYYRG | (bold) and His, tag |
| | QGTQVTVSSGGQHHHHHHGAYPYDVPDYAS | (SEQ ID NO: 8)/HA |
| | | tag (italics) |
| 16 | QVQLQESGGGLVQPGGSLRLSCTASGIALHSKATGWFRQA | Nanosota-9B VHH |
| | PGKEREGVSCISSGDGTTYYEDSVEGRFTISRDNAKNTVY | sequence with IgG1 |
| | LQMNSLKLEDTAVYYCAADPSGVCHSGSYYYTDDDFYYRG | Fc domain |
| | QGTQVTVSSEPKSCDKTHTCPPCPAPELLGGPSVFLFPPK | sequence (bold) |
| | PKDTLMISRTPEVTCVVVDVSHEDPEVKFNWYVDGVEVHN | |
| | AKTKPREEQYNSTYRVVSVLTVLHQDWLNGKEYKCKVSNK | |
| | ALPAPIEKTISKAKGQPREPQVYTLPPSREEMTKNQVSLT | |
| | CLVKGFYPSDIAVEWESNGQPENNYKTTPPVLDSDGSFFL | |
| | YSKLTVDKSRWQQGNVFSCSVMHEALHNHYTQKSLSLSPG | |
| | K | |
| 17 | QVQLQESGGGLVQPGGSLRLSCTASGIALHHLATGWERQA | Nanosota-9B-2 VHH |
| | PGKEREGVSCISSGDGTTYYEDSVEGRFTISRDNAKNTVY | sequence |
| | LQMNSLKLEDTAVYYCAADPVCHSGSYYYTDDDFYYRGQG | |
| | TQVTVSS | |
| 18 | TASGIALHHL | Nanosota-9B-2: CDR-H1 |
| 3 | ISSGDGTT | Nanosota-9B-2: CDR-H2 |
| 19 | DPVCHSGSYYYTDDDFYY | Nanosota-9B-2: CDR-H3 |
| 20 | EPKSCDKTHTCPPCPAPELLGGPSVFLFPPKPKDTLMISR | IgG1 Fc domain |
| | TPEVTCVVVDVSHEDPEVKFNWYVDGVEVHNAKTKPREEQ | sequence having |
| | YNSTYRVVSVLTVLHQDWLNGKEYKCKVSNKALPAPIEKT | T366Y mutation |
| | ISKAKGQPREPQVYTLPPSREEMTKNQVSLYCLVKGFYPS | (underlined) |
| | DIAVEWESNGQPENNYKTTPPVLDSDGSFFLYSKLTVDKS | |
| | RWQQGNVFSCSVMHEALHNHYTQKSLSLSPGK | |
| 21 | EPKSCDKTHTCPPCPAPELLGGPSVFLFPPKPKDTLMISR | IgG1 Fc domain |
| | TPEVTCVVVDVSHEDPEVKFNWYVDGVEVHNAKTKPREEQ | sequence having |
| | YNSTYRVVSVLTVLHQDWLNGKEYKCKVSNKALPAPIEKT | Y407T mutation |
| | ISKAKGQPREPQVYTLPPSREEMTKNQVSLTCLVKGFYPS | (underlined) |
| | DIAVEWESNGQPENNYKTTPPVLDSDGSFFLTSKLTVDKS | |
| | RWQQGNVFSCSVMHEALHNHYTQKSLSLSPGK | |
| 22 | CAGGTGCAGCTGCAGGAGTCTGGGGGAGGCTTGGTGCAGC | DNA sequence for |
| | CTGGGGGCTCTCTGAGACTCTCCTGTACAGCCTCTGGAAT | Nanosota-9B VHH |
| | CGCTTTGCATAGTAAGGCCACAGGCTGGTTCCGCCAGGCC | |
| | CCAGGGAAGGAGCGCGAGGGGGTCTCATGTATTAGTAGTG | |
| | GTGATGGTACCACATACTATGAAGACTCCGTGGAGGGGCG | |
| | ATTCACCATCTCCAGAGACAATGCCAAGAACACGGTGTAT | |
| | CTGCAAATGAACAGCCTGAAACTTGAGGATACGGCCGTTT | |
| | ATTACTGTGCGGCGGATCCAAGTGGGGTATGCCATAGTGG | |
| | CAGTTACTACTATACGGATGACGACTTCTACTACCGGGGG | |
| | CAGGGGACCCAGGTCACCGTCTCCTCA | |
| 23 | CAGGTGCAGCTGCAGGAGTCTGGGGGAGGCTTGGTGCAGC | DNA sequence for |
| | CTGGGGGCTCTCTGAGACTCTCCTGTACAGCCTCTGGAAT | Nanosota-9B-Fc |
| | CGCTTTGCATAGTAAGGCCACAGGCTGGTTCCGCCAGGCC | |
| | CCAGGGAAGGAGCGCGAGGGGGTCTCATGTATTAGTAGTG | |
| | GTGATGGTACCACATACTATGAAGACTCCGTGGAGGGGCG | |
| | ATTCACCATCTCCAGAGACAATGCCAAGAACACGGTGTAT | |
| | CTGCAAATGAACAGCCTGAAACTTGAGGATACGGCCGTTT | |
| | ATTACTGTGCGGCGGATCCAAGTGGGGTATGCCATAGTGG | |
| | CAGTTACTACTATACGGATGACGACTTCTACTACCGGGGG | |
| | CAGGGGACCCAGGTCACCGTCTCCTCAGAGCCCAAATCTT | |
| | GTGACAAAACTCACACATGCCCACCGTGCCCAGCACCTGA | |
| | ACTCCTGGGGGGACCGTCAGTCTTCCTCTTCCCCCCAAAA | |
| | CCCAAGGACACCCTCATGATCTCCCGGACCCCTGAGGTCA | |
| | CATGCGTGGTGGTGGACGTGAGCCACGAAGACCCTGAGGT | |
| | CAAGTTCAACTGGTACGTGGACGGCGTGGAGGTGCATAAT | |
| | GCCAAGACAAAGCCGCGGGAGGAGCAGTACAACAGCACGT | |
| | ACCGTGTGGTCAGCGTCCTCACCGTCCTGCACCAGGACTG | |
| | GCTGAATGGCAAGGAGTACAAGTGCAAGGTCTCCAACAAA | |
| | GCCCTCCCAGCCCCCATCGAGAAAACCATCTCCAAAGCCA | |
| | AAGGGCAGCCCCGAGAACCACAGGTGTACACCCTGCCCCC | |
| | ATCCCGGGAGGAGATGACCAAGAACCAGGTCAGCCTGACC | |
| | TGCCTGGTCAAAGGCTTCTATCCCAGCGACATCGCCGTGG | |
| | AGTGGGAGAGCAATGGGCAGCCGGAGAACAACTACAAGAC | |
| | CACGCCTCCCGTGCTGGACTCCGACGGCTCCTTCTTCCTC | |
| | TACAGCAAGCTCACCGTGGACAAGAGCAGGTGGCAGCAGG | |
| | GGAACGTCTTCTCATGCTCCGTGATGCATGAGGCTCTGCA | |
| | CAACCACTACACGCAGAAGAGCCTCTCCCTGTCTCCGGGT | |
| | AAA | |
| 24 | CAGGTGCAGCTGCAGGAGTCTGGGGGAGGCTTGGTGCAGC | DNA sequence for |
| | CTGGGGGCTCTCTGAGACTCTCCTGTACAGCCTCTGGAAT | Nanosota-9B-2 VHH |
| | CGCTTTGCATCATTTGGCCACAGGCTGGTTCCGCCAGGCC | |
| | CCAGGGAAGGAGCGCGAGGGGGTCTCATGTATTAGTAGTG | |
| | GTGATGGTACCACATACTATGAAGACTCCGTGGAGGGGCG | |
| | ATTCACCATCTCCAGAGACAATGCCAAGAACACGGTGTAT | |
| | CTGCAAATGAACAGCCTGAAACTTGAGGATACGGCCGTTT | |
| | ATTACTGTGCGGCGGATCCGGTATGCCATAGTGGCAGTTA | |
| | CTACTATACGGATGACGACTTCTACTACCGGGGCCAGGGG | |
| | ACCCAGGTCACCGTCTCCTCA | |
| 25 | TASGIALHX₁X₂ | X₁ is T, S, or H, |
| | | X₂ is H, K, or L |
| 26 | DPX₃X₄VCHSGSYYYTDDDFYY | X₃ is G, S, or absent, |
| | | X₄ is A, G, or absent |
| 27 | QVQLQESGGGLVQPGGSLRLSCTASGIALHTHATGWFRQA | Nanosota-9A VHH |
| | PGKEREGVSCISSGDGTTYYEDSVEGRFTISRDNAKNTVY | sequence with IgG1 |
| | LQMNSLKLEDTAVYYCAADPGAVCHSGSYYYTDDDFYYRG | Fc domain |
| | QGTQVTVSSEPKSCDKTHTCPPCPAPELLGGPSVFLFPPK | sequence (bold ) having |
| | PKDTLMISRTPEVTCVVVDVSHEDPEVKFNWYVDGVEVHN | T366Y mutation |
| | AKTKPREEQYNSTYRVVSVLTVLHQDWLNGKEYKCKVSNK | (bold/underlined) |
| | ALPAPIEKTISKAKGQPREPQVYTLPPSREEMTKNQVSLY | |
| | CLVKGFYPSDIAVEWESNGQPENNYKTTPPVLDSDGSFFL | |
| | YSKLTVDKSRWQQGNVFSCSVMHEALHNHYTQKSLSLSPG | |
| | K | |
| 28 | QVQLQESGGGLVQPGGSLRLSCTASGIALHSKATGWFRQA | Nanosota-9B VHH |
| | PGKEREGVSCISSGDGTTYYEDSVEGRFTISRDNAKNTVY | sequence with IgG1 |
| | LQMNSLKLEDTAVYYCAADPSGVCHSGSYYYTDDDFYYRG | Fc domain |
| | QGTQVTVSSEPKSCDKTHTCPPCPAPELLGGPSVFLFPPK | sequence (bold ) having |
| | PKDTLMISRTPEVTCVVVDVSHEDPEVKFNWYVDGVEVHN | Y407T mutation |
| | AKTKPREEQYNSTYRVVSVLTVLHQDWLNGKEYKCKVSNK | (bold/underlined) |
| | ALPAPIEKTISKAKGQPREPQVYTLPPSREEMTKNQVSLT | |
| | CLVKGFYPSDIAVEWESNGQPENNYKTTPPVLDSDGSFFL | |
| | TSKLTVDKSRWQQGNVFSCSVMHEALHNHYTQKSLSLSPG | |
| | K | |
| 29 | QVQLQESGGGLVQPGGSLRLSCTASGIALHHLATGWFRQA | Nanosota-9B-2 VHH |
| | PGKEREGVSCISSGDGTTYYEDSVEGRFTISRDNAKNTVY | sequence with IgG1 |
| | LQMNSLKLEDTAVYYCAADPVCHSGSYYYTDDDFYYRGQG | Fc domain |
| | TQVTVSSEPKSCDKTHTCPPCPAPELLGGPSVFLFPPKPK | sequence (bold) |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSREEMTKNQVSLTCL | |
| | VKGFYPSDIAVEWESNGQPENNYKTTPPVLDSDGSFFLYS | |
| | KLTVDKSRWQQGNVFSCSVMHEALHNHYTQKSLSLSPGK | |

The invention will now be illustrated by the following non-limiting Embodiments and Examples.

Embodiment 1. An isolated anti-SARS-CoV-2 binder protein comprising: one or more CDRs selected from the group consisting of:
(a) a CDR1 comprising an amino acid sequence having at least 80% sequence identity to the amino acid sequence of TASGIALHTH (SEQ ID NO:2);
(b) a CDR2 comprising an amino acid sequence having at least 80% sequence identity to the amino acid sequence of ISSGDGTT (SEQ ID NO:3); and
(c) a CDR3 comprising an amino acid sequence having at least 80% sequence identity to the amino acid sequence of DPGAVCHSGSYYYTDDDFYY (SEQ ID NO:4).

Embodiment 2. The isolated anti-SARS-CoV-2 binder protein of Embodiment 1, comprising one or more CDRs selected from the group consisting of:
(a) a CDR1 comprising an amino acid sequence having at least 90% sequence identity to the amino acid sequence of TASGIALHTH (SEQ ID NO:2);
(b) a CDR2 comprising an amino acid sequence having at least 90% sequence identity to the amino acid sequence of ISSGDGTT (SEQ ID NO:3); and
(c) a CDR3 comprising an amino acid sequence having at least 90% sequence identity to the amino acid sequence of DPGAVCHSGSYYYTDDDFYY (SEQ ID NO:4).

Embodiment 3. The isolated anti-SARS-CoV-2 binder protein of any one of Embodiments 1-2, comprising:
(a) a CDR1 comprising the amino acid sequence of TASGIALHTH (SEQ ID NO:2);
(b) a CDR2 comprising the amino acid sequence of ISSGDGTT (SEQ ID NO:3); and
(c) a CDR3 comprising the amino acid sequence of DPGAVCHSGSYYYTDDDFYY (SEQ ID NO: 4).

Embodiment 4. The isolated anti-SARS-CoV-2 binder protein of any one of Embodiments 1-3, comprising an amino acid sequence that has at least 80% sequence identity to

(SEQ ID NO: 1)
QVQLQESGGGLVQPGGSLRLSCTASGIALHTHATGWFRQAPGKEREGVS
CISSGDGTTYYEDSVEGRFTISRDNAKNTVYLQMNSLKLEDTAVYYCAA
DPGAVCHSGSYYYTDDDFYYRGQGTQVTVSS.

Embodiment 5. The isolated anti-SARS-CoV-2 binder protein of any one of Embodiments 1-4, comprising an amino acid sequence that has at least 90% sequence identity to SEQ ID NO:1.

Embodiment 6. The isolated anti-SARS-CoV-2 binder protein of any one of Embodiments 1-5, comprising an amino acid sequence that has at least 95% sequence identity to SEQ ID NO:1.

Embodiment 7. The isolated anti-SARS-CoV-2 binder protein of any one of Embodiments 1-6, comprising an amino acid sequence that has at least 99% sequence identity to SEQ ID NO:1.

Embodiment 8. The isolated anti-SARS-CoV-2 binder protein of any one of Embodiments 1-7, comprising the amino acid sequence of SEQ ID NO:1.

Embodiment 9. The isolated anti-SARS-CoV-2 binder protein of any one of Embodiments 1-8, wherein the binder protein comprises an anti-SARS-CoV-2 single-domain antibody (sdAb).

Embodiment 10. The isolated anti-SARS-CoV-2 binder protein of any one of Embodiments 1-9, wherein the binder protein comprises an anti-SARS-CoV-2 single-domain antibody (sdAb) that is linked to at least one polypeptide tag through a peptide bond or a polypeptide linker.

Embodiment 11. The isolated anti-SARS-CoV-2 binder protein of Embodiment 10, wherein the at least one polypeptide tag is operably linked to the C-terminus of the sdAb.

Embodiment 12. The isolated anti-SARS-CoV-2 binder protein of any one of Embodiments 10-11, wherein the at least one polypeptide tag comprises a His tag (e.g., a His6 tag (SEQ ID NO: 8)), HA tag, Myc tag, or Fc tag.

Embodiment 13. The isolated anti-SARS-CoV-2 binder protein of any one of Embodiments 10-12, wherein the at least one polypeptide tag comprises a Fc tag.

Embodiment 14. The isolated anti-SARS-CoV-2 binder protein of Embodiment 13, wherein the Fc tag comprises a human IgG1, IgG2, IgG3, or IgG4 Fc domain amino acid sequence.

Embodiment 15. The isolated anti-SARS-CoV-2 binder protein of Embodiment 14, wherein the Fc tag comprises an amino acid sequence having at least 80% sequence identity to SEQ ID NO:9.

Embodiment 16. The isolated anti-SARS-CoV-2 binder protein of Embodiment 15, comprising an amino acid sequence that has at least 90% sequence identity to:

(SEQ ID NO: 6)
QVQLQESGGGLVQPGGSLRLSCTASGIALHTHATGWFRQAPGKEREGVS
CISSGDGTTYYEDSVEGRFTISRDNAKNTVYLQMNSLKLEDTAVYYCAA
DPGAVCHSGSYYYTDDDFYYRGQGTQVTVSSEPKSCDKTHTCPPCPAPE
LLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMH
EALHNHYTQKSLSLSPGK.

Embodiment 17. The isolated anti-SARS-CoV-2 binder protein of Embodiment 16, comprising the amino acid sequence of SEQ ID NO:6.

Embodiment 18. A pharmaceutical composition comprising the isolated anti-SARS-CoV-2 binder protein according to any one of Embodiments 1-17, and a pharmaceutically acceptable carrier.

Embodiment 19. An isolated polynucleotide comprising a nucleotide sequence encoding an isolated anti-SARS-CoV-2 binder protein of any one of Embodiments 1-17.

Embodiment 20. A vector comprising the polynucleotide of Embodiment 19.

Embodiment 21. A cell comprising the polynucleotide of Embodiment 19 or the vector of Embodiment 20.

Embodiment 22. A method for treating or preventing a SARS-CoV-2 infection in a mammal, comprising administering an effective amount of an isolated anti-SARS-CoV-2 binder protein of any one of Embodiments 1-17 to the mammal.

### EXAMPLE 1. DISCOVERY OF NANOSOTA-9 AS ANTI-OMICRON NANOBODY THERAPEUTIC CANDIDATE

Omicron subvariants of SARS-CoV-2 continue to pose a significant global health threat. Nanobodies, single-domain antibodies derived from camelids, are promising therapeutic tools against pandemic viruses due to their favorable properties. In this study, we identified a novel nanobody, Nanosota-9, which demonstrates high potency against a wide range of Omicron subvariants both in vitro and in a mouse model. Cryo-EM data revealed that Nanosota-9 neutralizes Omicron through a unique mechanism: two Nanosota-9 molecules crosslink two receptor-binding domains (RBDs) of the trimeric Omicron spike protein, preventing the RBDs from binding to the ACE2 receptor. This mechanism explains its strong anti-Omicron potency. Additionally, the Nanosota-9 binding epitopes on the spike protein are relatively conserved among Omicron subvariants, contributing to its broad anti-Omicron spectrum. Combined with our recently developed structure-guided in vitro evolution approach for nanobodies, Nanosota-9 has the potential to serve as the foundation for a superior anti-Omicron therapeutic.

### INTRODUCTION

Omicron subvariants of SARS-CoV-2 continue to circulate among human populations, posing a threat to global health. Since Omicron's emergence in the fall of 2021, new Omicron subvariants have continually appeared, from the earliest BA.1 to later BA.5, and more recent ones like XBB.1.5 and JN.1, up to the current KP.2 and KP.3 [1, 2]. It is likely that Omicron will persist in human populations long-term, necessitating ongoing interventions, including vaccines and antiviral therapeutics. Although SARS-CoV-2 vaccines are available, breakthrough infections are common [3, 4]. While several anti-SARS-CoV-2 therapeutics exist, Paxlovid is the most effective and widely used. Paxlovid is a combination of a protease inhibitor and a helper drug that inhibits liver function, raising concerns about potential drug-drug interactions and liver and kidney toxicity [5, 6]. Moreover, relying on a single drug increases the risk of viral escape mutations [7]. Therefore, novel anti-Omicron therapeutics are urgently needed to effectively control Omicron.

Antibodies are among the primary antiviral therapeutics due to their high target specificity and hence high safety [8]. The SARS-CoV-2 spike protein is the top target for antibody therapeutics [9, 10]. It is a homotrimer on the virus surface, having three copies of the receptor-binding S1 subunit and a trimeric membrane-fusion S2 stalk. During viral entry, the receptor-binding domain (RBD) in S binds to its host receptor ACE2 on the cell surface, the spike protein is cleaved by host proteases at the S1/S2 boundary, and S2 undergoes a dramatic structural change to fuse the viral and host membranes. Early in the pandemic, we discovered three key structural mechanisms of the SARS-CoV-2 spike protein: its RBD has a high affinity for human ACE2, its RBD can either stand up for receptor binding or lie down for immune evasion, and it can be cleaved by the human protease furin [11, 12]. Because the RBD induces most of the neutralizing immune responses in humans, it is heavily targeted by antibody therapeutics. The RBD contains two subdomains: a core structure and a receptor-binding motif (RBM), with the RBM mediating receptor binding [10, 13]. RBD-targeting antibody therapeutics need to bind to the RBM with very high affinity, blocking ACE2 binding. Ideally, they should also target both the standing up and lying down RBDs to counter viral evasion. Indeed, the RBD is the primary target of most neutralizing antibody responses [14]. However, human antibody therapeutics have faced setbacks against SARS-CoV-2 due to the extensive evolution of the RBD [15, 16]. Currently, there are no published antibody treatments for the latest KP.2 and KP.3 Omicron subvariants. Importantly, because of their large size, human antibodies cannot easily access conserved cryptic epitopes [17, 18], limiting their potency and antiviral spectrum. Small-sized antibodies are potential solutions as anti-SARS-CoV-2 entry inhibitors.

Nanobodies are single-domain antibodies derived from camelid animals [19-21]. Due to their small size, they possess excellent therapeutic properties as antiviral drugs. They have superior epitope accessibility and tissue permeability, which contribute to their high potency. They can access conserved hidden epitopes, providing them with broad antiviral spectrums. Nanobodies are easy to produce, transport, and store, making them cost-effective. They can potentially be administered intranasally, offering an attractive needle-free therapy option [22, 23]. Additionally, they demonstrate minimal toxicity and immunogenicity in humans [24, 25]. A nanobody drug is clinically available to treat a blood clotting disorder [26], validating the safety of nanobodies as human therapeutics. Numerous nanobody inhibitors have been developed against pre-Omicron variants and early Omicron subvariants [21]. However, due to the extensive evolution of Omicron, there is an urgent need for novel nanobody inhibitors against recent and current Omicron subvariants to counter the spread of Omicron. So far, only one study has identified a nanobody inhibitor against the JN.1 subvariant, but it was only tested in vitro, without any in vivo or structural data [27]. No nanobody has been developed against the KP.2 or KP.3 subvariant. Therefore, broad-spectrum nanobody inhibitors against recent and current Omicron subvariants with demonstrated in vivo efficacy are yet to be discovered.

Previously, we developed several nanobody inhibitors, collectively named the Nanosota series, against the prototypic SARS-CoV-2 spike [28-30]. More recently, we introduced a novel structure-guided in vitro evolution approach to rapidly adapt nanobodies to emerging SARS-CoV-2 variants [31]. In this study, we discovered a novel nanobody, named Nanosota-9, from an alpaca immunized with the Omicron BA.5 spike protein. Nanosota-9 exhibits high anti-Omicron potency and a broad anti-Omicron spectrum. It not only possesses many of the ideal qualities mentioned above, but also utilizes a unique structural mechanism to inhibit Omicron. Combined with our structure-guided in vitro evolution approach, Nanosota-9 holds great potential as the foundation for a superior anti-Omicron therapeutic.

### Results

Discovery of Nanosota-9 from Immunized Alpaca

To discover anti-Omicron nanobodies, we immunized an alpaca with the recombinant spike ectodomain of the Omicron subvariant BA.5. We collected peripheral blood mononuclear cells (PBMCs) from the immunized alpaca and established an induced nanobody phage display library. We then screened this library using the recombinant BA.5 spike ectodomain as bait. Through this process, we discovered a nanobody named Nanosota-9. We constructed a human Fc-tagged version of Nanosota-9, named Nanosota-9-Fc. As previously shown, compared to monomeric nanobodies, Fc-tagged nanobodies have significantly enhanced in vivo half-lives partly due to surpassing the size threshold for kidney clearance [29]. Nevertheless, the sizes of Fc-tagged nanobodies are still half that of human antibodies. Importantly, Fc-tagged nanobodies maintain the single-domain structure for target binding, which is crucial for binding cryptic epitopes. Therefore, Nanosota-9-Fc was used in this study to evaluate its anti-Omicron activities, while Nanosota-9-His (His-tagged Nanosota-9) was used only for structural studies.

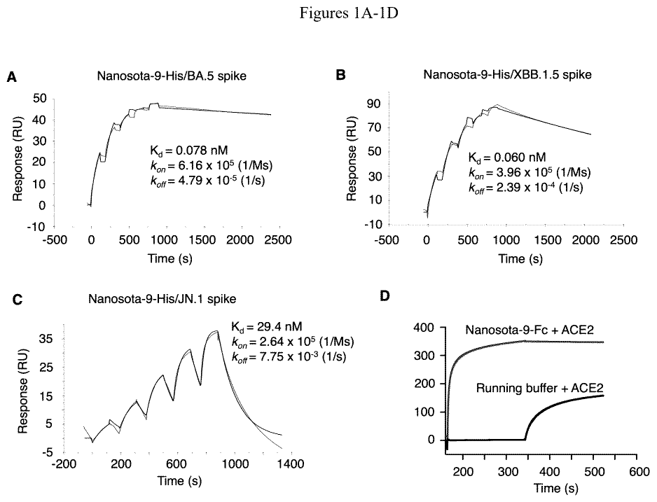

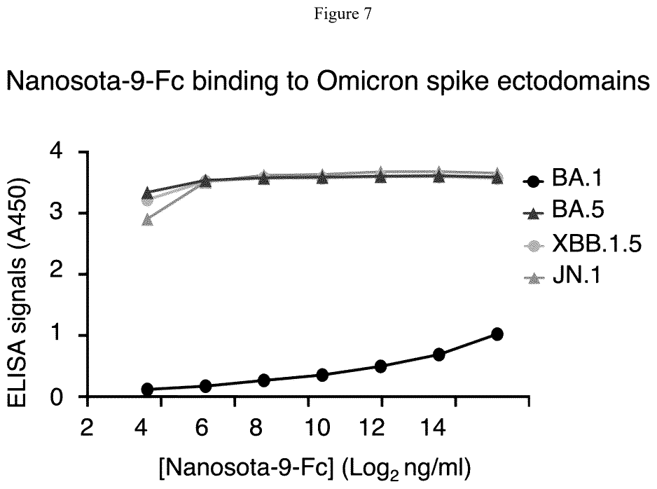

We characterized the binding of Nanosota-9-Fc to its targets through several assays. First, we conducted an ELISA to evaluate the binding of Nanosota-9-Fc to recombinant spike ectodomains from different Omicron subvariants. The results showed that Nanosota-9-Fc binds with high affinity to the spike ectodomains from the BA.5, XBB.1.5, and JN.1 subvariants, but does not bind to the spike ectodomain from the BA.1 subvariant as tightly as to the spike of BA.5, XBB.1.5, and JN.1 (FIG. 7). Second, we used surface plasmon resonance (SPR) to measure the binding affinity between Nanosota-9-His and the spike ectodomains from the BA.5, XBB.1.5, and JN.1 subvariants (FIGS. 1A, 1B, 1C). The results indicated that Nanosota-9-His exhibits tight binding to all three spike ectodomains, with dissociation constants (Kds) of 0.078 nM, 0.060 nM, and 29.4 nM for the BA.5, XBB. 1.5, and JN.1 spikes, respectively. Third, we carried out competitive SPR to investigate potential competition between Nanosota-9 and ACE2 for binding to the XBB.1.5 spike ectodomain (FIG. 1D). The results showed that Nanosota-9 and ACE2 do not bind the XBB.1.5 spike protein simultaneously. Together, these data indicate that Nanosota-9 potently targets the spikes of BA.5, XBB.1.5, and JN.1, but not BA.1, and that Nanosota-9 binds to overlapping epitopes on the spikes with ACE2.

### Neutralizing Potency of Nanosota-9 Against Omicron

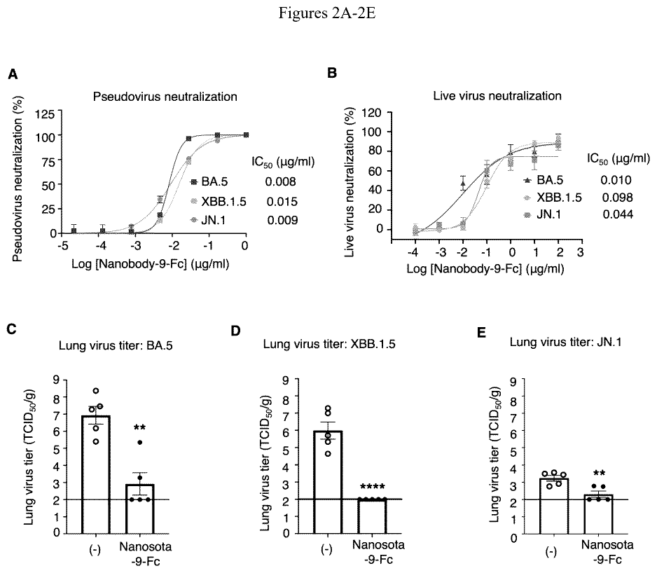

We evaluated the neutralizing potency of Nanosota-9 against Omicron using two different in vitro assays. First, we conducted an Omicron pseudovirus entry assay. Lentiviruses pseudotyped with each of the Omicron spikes (i.e., Omicron pseudoviruses) were used to enter human ACE2-expressing cells in the presence of Nanosota-9-Fc. The results showed that Nanosota-9-Fc potently neutralized the entry of all three Omicron pseudoviruses, BA.5, XBB.1.5, and JN.1, with IC₅₀ values of 8 ng/ml, 15 ng/ml, and 9 ng/ml, respectively (FIG. 2A). Second, we performed a live Omicron infection assay, where each of the live Omicron viruses was used to infect ACE2-expressing cells in the presence of Nanosota-9-Fc. The results showed that Nanosota-9-Fc potently neutralized the infection of all three Omicron subvariants, BA.5, XBB.1.5, and JN.1, with IC₅₀ values of 10 ng/ml, 98 ng/ml, and 44 ng/ml, respectively (FIG. 2B). Thus, Nanosota-9-Fc is a potent neutralizer of all three Omicron subvariants in vitro.

We also evaluated the neutralizing potency of Nanosota-9 against Omicron in vivo. Mice were challenged with each of the three Omicron subvariants, BA.5, XBB.1.5, and JN.1, through intranasal inoculation. For each subvariant, the mice were divided into two groups: four hours post-challenge, one group received Nanosota-9-Fc at a dosage of 10 mg/kg via the intraperitoneal route, while the other group received PBS buffer. All six groups of mice were monitored for lung virus titers two days post-challenge. The results showed that both the BA.5 and XBB.1.5 subvariants replicated efficiently in the mice, reaching titers of 10⁷ TCID₅₀ and 106 TCID₅₀ in the lungs on day 2, respectively (FIGS. 2C-2D). Nanosota-9-Fc reduced the titers of both BA.5 and XBB.1.5 in the lungs by 10,000-fold, with the XBB. 1.5 titer reduced to near the detection limit (FIGS. 2C-2D). However, the JN.1 subvariant replicated inefficiently in the mice, reaching a titer of only 10³ TCID₅₀ in the lungs on day 2 (FIG. 2E), suggesting that JN.1 differs from BA.5 and XBB.1.5 in their replication efficiency in the current mouse model. Nevertheless, Nanosota-9-Fc significantly reduced the JN.1 titer in the lungs to near the detection limit (FIG. 2E). Therefore, Nanosota-9-Fc is a potent neutralizer of all three Omicron subvariants in vivo.

### Structural Basis for Anti-Omicron Efficacy of Nanosota-9

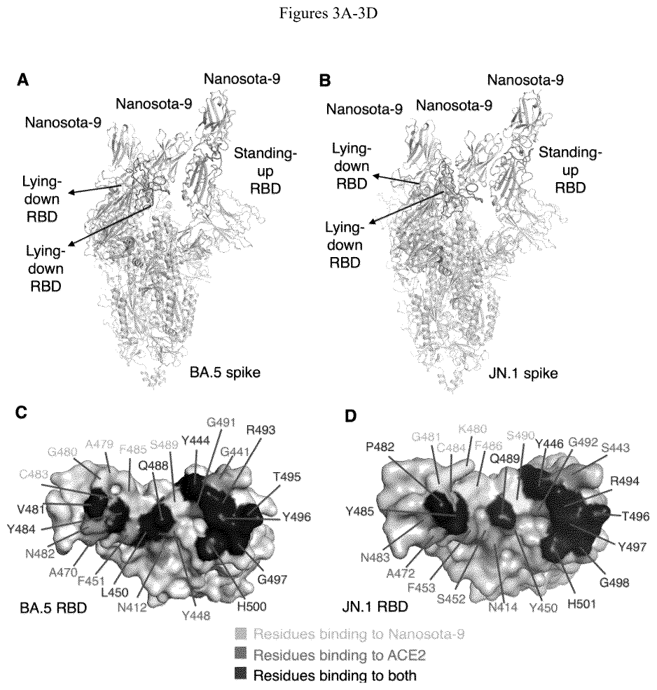

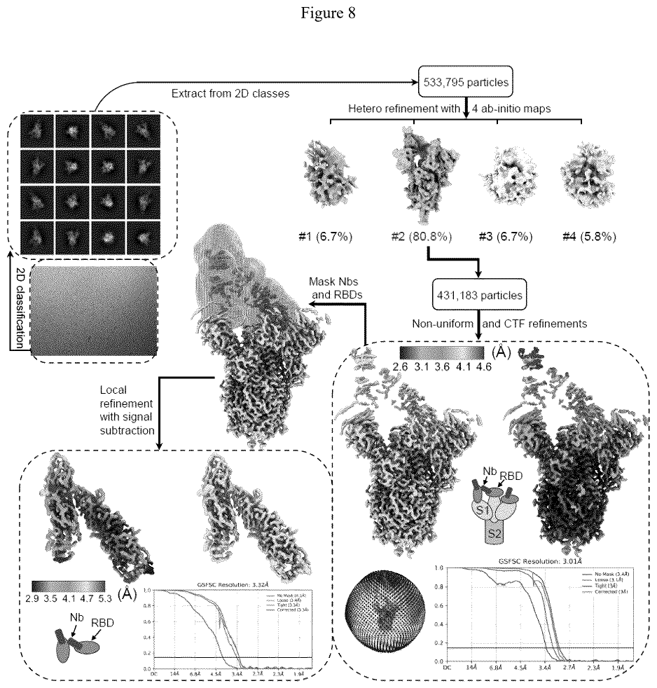

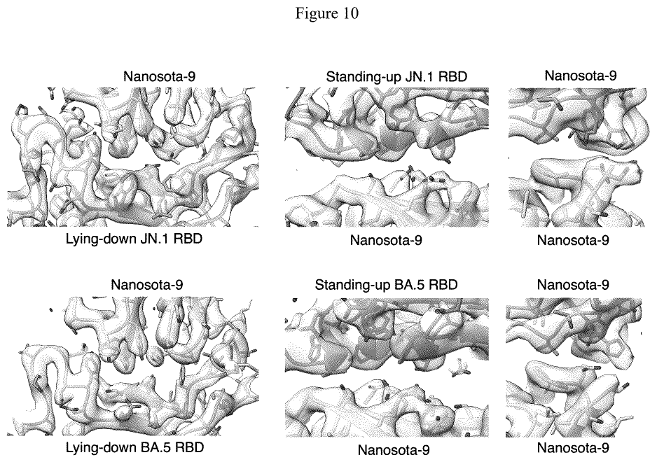

To understand the structural basis of Nanosota-9's potency against Omicron, we determined the cryo-EM structures of the BA.5 and JN.1 spike ectodomains complexed with Nanosota-9 (FIGS. 3A-3B; FIGS. 8, 9, 10; Table S1). The structures revealed that Nanosota-9 binds to both spikes in a similar manner: it engages all three copies of the RBD, with one in a “standing-up” conformation and the other two in a “lying-down” conformation. The structural interfaces between Nanosota-9 and the BA.5 and JN.1 RBDs are also highly similar (FIGS. 3C-3D, FIG. 4, FIG. 11). Nanosota-9 binds to the same epitope on the RBDs, which significantly overlaps with the ACE2 binding site (FIGS. 3C-3D). Specifically, Nanosota-9 directly contacts 15 BA.5 RBD residues, while ACE2 directly contacts 17 BA.5 RBD residues, with 10 residues shared between both. This result aligns with the competitive SPR findings, which showed that Nanosota-9 and ACE2 cannot bind to the spike protein simultaneously (FIG. 1D). Therefore, similar to Nanosota-1, -2, -3, and -4, Nanosota-9 neutralizes viral entry by blocking the spike protein's receptor binding.

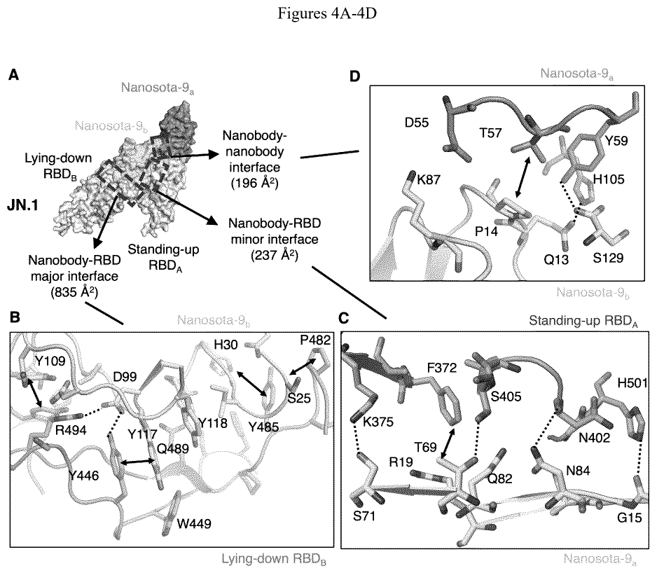

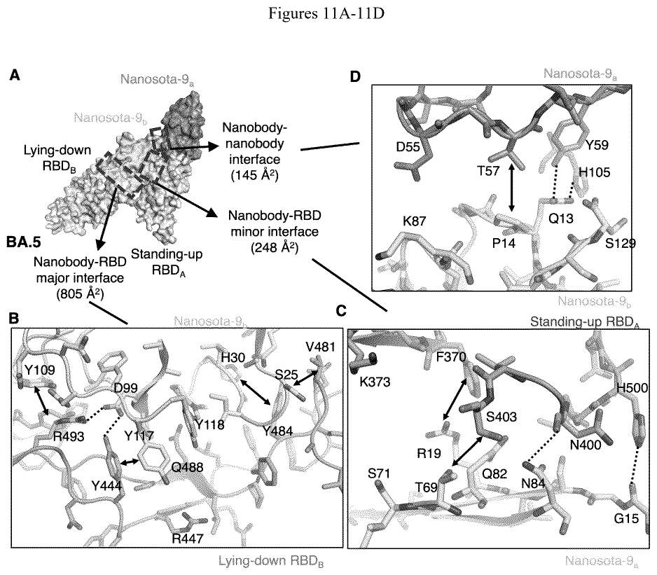

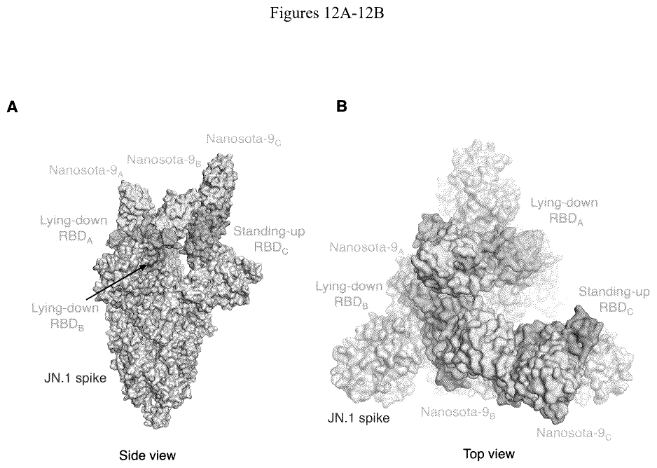

Nanosota-9 exhibits a unique binding mode with Omicron spikes. While three Nanosota-9 molecules bind to each trimeric spike (which has three RBDs), their distribution is asymmetrical: two of the three Nanosota-9 molecules crosslink two RBDs, connecting one standing-up RBD with one lying-down RBD. This contrasts with Nanosota-1, -2, -3, and -4, each of which binds to an RBD in a one-to-one manner. The 2:2 binding mode of Nanosota-9 with the RBD creates three distinct interfaces (FIG. 4A, FIG. 11A, FIG. 12). The major interface between Nanosota-9 and the RBM buries 835 Å² of surface area and is dominated by strong hydrophobic stacking interactions (FIG. 4B, FIG. 11B). The minor interface involves the RBD core, burying 237 Å² primarily through hydrogen bonds (FIG. 4C, FIG. 11C). Additionally, the interface between two Nanosota-9 molecules buries 196 Å², featuring fewer interactions than the other two interfaces (FIG. 4D, FIG. 11D). Due to the relatively small sizes of the latter two interfaces, the 2:2 binding mode of Nanosota-9 with the RBD may only occur in the context of the trimeric spike. Collectively, these three interfaces stabilize the Nanosota-9/spike complex by locking one RBD in the “lying-down” position and the other in the “standing-up” position, rendering both RBDs' ACE2-binding sites inaccessible. This unique binding mode enhances Nanosota-9's anti-Omicron potency.

### Anti-Omicron Spectrum of Nanosota-9

To understand the anti-Omicron spectrum of Nanosota-9, we analyzed the evolution of Omicron subvariants at the Nanosota-9-binding interfaces. Sequence alignment of the RBDs from BA.5, XBB.1.5, and JN.1 revealed three mutations in residues that interact directly with Nanosota-9:478, 480, and 486 (FIG. 5A). However, these residues interact through their main-chain functional groups, so side-chain changes likely have little effect on the binding of Nanosota-9 (FIG. 5B, 5C, 5D). This explains why Nanosota-9 effectively neutralizes BA.5, XBB.1.5, and JN.1. Furthermore, only one residue in contact with Nanosota-9 mutated between BA.1 and the other subvariants: residue 489 (FIG. 5E). In the JN.1 RBD, Gln489 forms two hydrogen bonds with Thr31 of Nanosota-9, involving both the side-chain hydroxyl group and the main-chain carbonyl oxygen of Thr31 (FIG. 5E). The Q489R mutation likely disrupts these favorable interactions and introduces clashes between Arg489's long side chain and Nanosota-9, explaining Nanosota-9's inability to neutralize BA.1. These structural insights align with biochemical and virological findings on Nanosota-9's antiviral effectiveness against earlier and recent Omicron subvariants.

We extended this analysis to two currently circulating subvariants, KP.2 and KP.3. Sequence alignment of the RBDs from JN.1, KP.2, and KP.3 revealed two mutations in KP.2 (residues 343 and 453) and two in KP.3 (residues 453 and 489) (FIG. 5A, 5F, 5G). Residues 343 and 453 do not interact directly with Nanosota-9, so these changes likely do not affect its binding to KP.2 RBD. However, in the KP.3 RBD, the Q489E mutation likely maintains a hydrogen bond with Thr31's side chain but introduces an unfavorable interaction between the negatively charged side chain of Glu489 and the partially negatively charged main-chain carbonyl oxygen of Thr31 (FIG. 5H). As a result, similar to BA.1, Nanosota-9 likely fails to neutralize KP.3 due to the altered residue at position 489. With detailed structural data now available for the RBD/Nanosota-9 interfaces, our newly developed structure-guided in vitro evolution approach can help evolve Nanosota-9 to overcome the Q489E mutation [31], potentially enabling neutralization of KP.3.

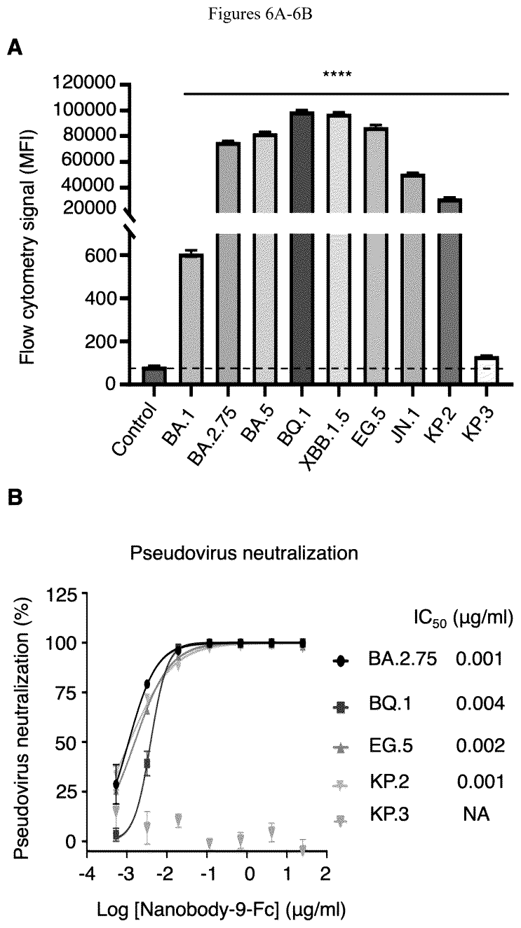

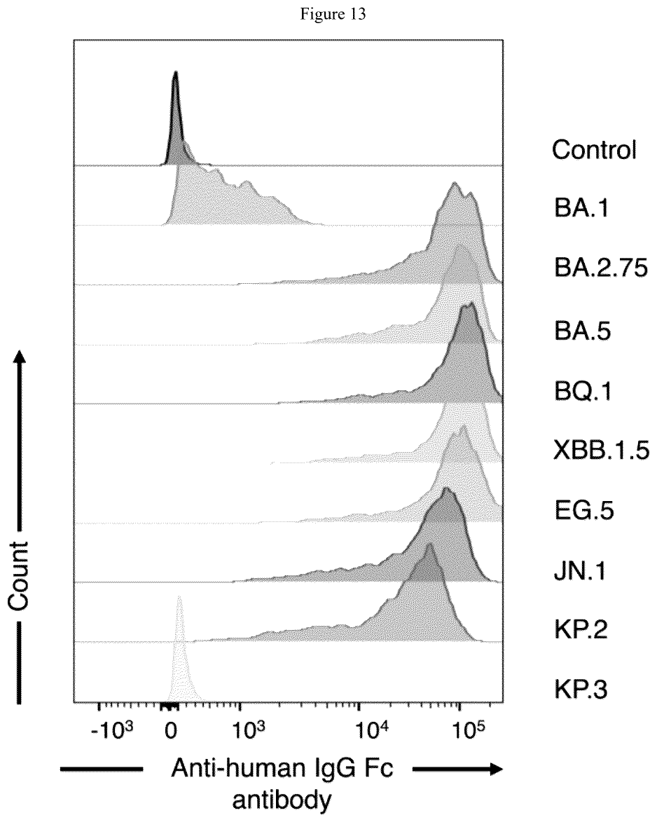

To validate the above structural analysis, we performed two biochemical assays to extensively assess Nanosota-9's anti-Omicron spectrum. First, using flow cytometry, we tested Nanosota-9's binding to cell-surface-expressed spike proteins from nine Omicron subvariants: BA.1, BA.2.75, BA.5, BQ.1, XBB. 1.5, EG.5, JN.1, KP.2, and KP.3. The results showed high-affinity binding to all subvariants except BA.1 and KP.3 (FIG. 6A; FIG. 13). Next, we conducted a pseudovirus neutralization assay to examine Nanosota-9's potency against the same subvariants (except BA.5, XBB.1.5, and JN.1, which were measured earlier; FIG. 2A). Again, Nanosota-9 neutralized all except BA.1 and KP.3 with high potency (FIG. 6B). Thus, the biochemical data corroborate the structural analysis, indicating that Nanosota-9 has broad anti-Omicron coverage, except for BA.1 and KP.3, likely due to the mutation at position 489 in their RBDs. Structure-guided in vitro evolution of Nanosota-9 can help overcome this mutation and further expand its anti-Omicron spectrum.

### DISCUSSION

Current intervention strategies against Omicron have significant limitations, underscoring the urgent need for novel inhibitors. Due to the constant evolution of Omicron, all existing entry inhibitors have struggled to keep pace: currently, there are no published antibodies or nanobody inhibitors effective against the current KP.2 and KP.3 subvariants. Although the RBD has the highest mutation rate compared to other regions of the spike protein, RBD-targeting antibodies remain the most dominant and potent compared to those targeting other spike regions [14]. We recently developed a structure-guided in vitro evolution approach to rapidly adapt nanobodies to emerging viral variants [31], offering hope that entry inhibitors can keep up with Omicron's evolution. In this study, we identified a novel nanobody, Nanosota-9, which targets a relatively conserved epitope on the Omicron RBD and effectively neutralizes the majority of main Omicron subvariants. With its unique mechanism, potent neutralization, and broad spectrum, Nanosota-9 stands out as a promising therapeutic candidate against Omicron, especially when combined with the new nanobody evolution strategy.

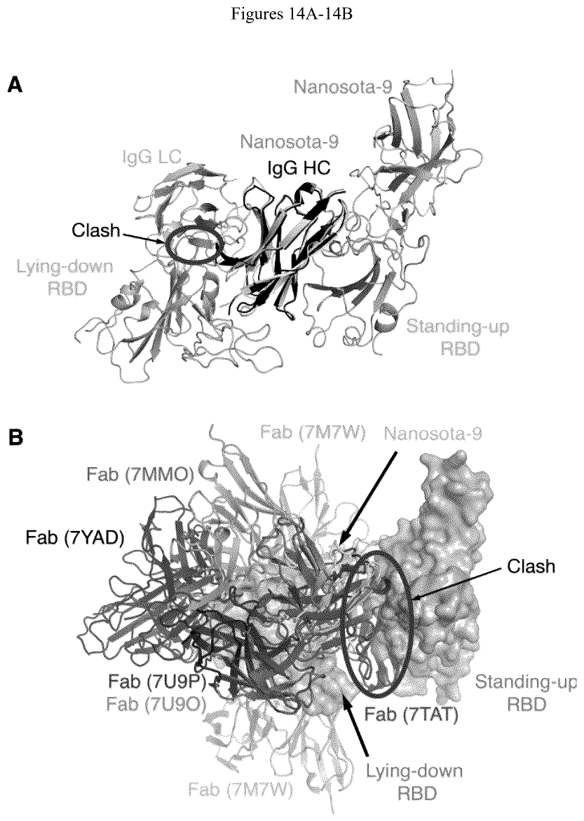

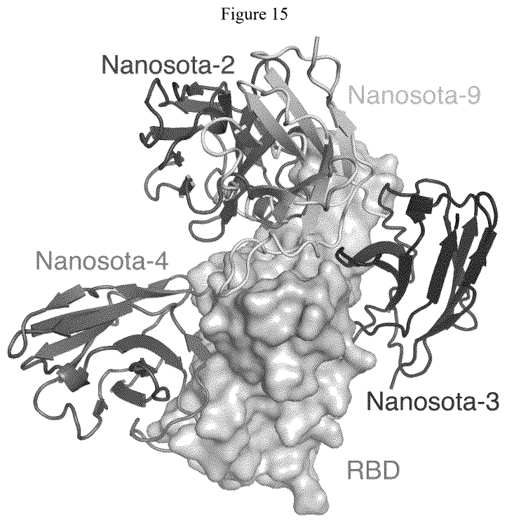

The neutralizing potency of Nanosota-9 against Omicron stems from its unique structural mechanisms in binding to the Omicron spike proteins. First, Nanosota-9's primary binding site on the RBD heavily overlaps with the ACE2-binding site, suggesting that Nanosota-9 neutralizes Omicron by blocking receptor binding [12]. Second, Nanosota-9 binds to both the “standing up” and “lying down” RBDs, effectively countering viral evasion [11]. Third, two Nanosota-9 molecules crosslink two RBDs together, locking one of the two RBDs in the “lying down” position, preventing it from binding to the ACE2 receptor and enhancing the anti-Omicron potency of Nanosota-9. Docking a human antibody into the same binding pocket as Nanosota-9 revealed clashes between the human antibody and the spike protein (FIG. 14A), indicating that this binding site is not accessible to human antibodies. Furthermore, comparing the Nanosota-9 epitope with those of human antibodies targeting the Omicron RBD revealed that only one human antibody shares an overlapping epitope (FIG. 14B). However, if this human antibody were to bind to a lying-down RBD, it would clash with a standing-up RBD (FIG. 14B), confirming that the Nanosota-9 binding site is inaccessible to human antibodies. Hence, this crosslinking strategy represents a unique antiviral mechanism for nanobodies. Comparison of the epitopes of Nanosota-9 to those of Nanosota-2, -3, and -4 reveals that the Nanosota-9 epitopes overlap with but also differ from those of Nanosota-2 and -3 (FIG. 15), which explains why only Nanosota-9, but not the other nanobodies, can crosslink the RBDs. These structural mechanisms collectively explain the neutralizing potency of Nanosota-9 against Omicron.

The broad spectrum of Nanosota-9 against Omicron is due to the structural features of the interfaces that it forms with Omicron RBDs. Nanosota-9 forms two binding interfaces with the RBD. The minor interface with the core of the RBD is completely conserved among recent and current Omicron subvariants likely due to its inaccessibility to human antibodies. The major interface with the RBM of the RBD heavily overlaps with the ACE2-binding site, restricting the virus's ability to mutate many of the dual binding RBD residues. Consequently, the major epitope is relatively conserved among recent and current Omicron subvariants. Although a few mutations have occurred at the major epitope among different Omicron subvariants, detailed structural analysis suggests that they are unlikely to significantly impact the RBDs' binding to Nanosota-9. The relative conservation of the Nanosota-9 major epitope is also likely due to its inaccessibility to human antibodies when the RBD is in the lying-down position. However, since the major epitope remains accessible to human antibodies when the RBD is in the standing-up position, it is not fully conserved. As demonstrated in this study, while Nanosota-9 effectively neutralizes most major Omicron subvariants, a single mutation at residue 489 likely reduced its potency against BA.1 and KP.3. Despite this, the restricted accessibility to human antibodies helps maintain the relative conservation of the major epitope. Moreover, if a limited number of mutations occur within this epitope, our newly developed structure-guided in vitro evolution approach can help adapt Nanosota-9 to keep pace with the evolving Omicron subvariants at this site.

In summary, Nanosota-9 possesses several properties that make it an ideal anti-Omicron therapeutic candidate. It directly blocks ACE2 binding to the RBD and neutralizes viral entry. By binding to both “standing-up” and “lying-down” RBDs, it counteracts viral evasion. Nanosota-9 employs a unique crosslinking mechanism to further inhibit ACE2 binding and enhance antiviral potency. The interfaces it forms with the RBD are relatively conserved among Omicron subvariants. Beyond these structural features, our previous research has identified other advantageous properties of nanobodies that are likely applicable to Nanosota-9. For example, nanobodies' high expression yield and in vitro thermostability make them cost-effective [29], and their potential for intranasal administration offers an appealing needle-free therapy option [30], in addition to traditional injections. Our earlier studies also demonstrated that nanobodies are significantly more effective when used prophylactically than therapeutically [29, 30]. Therefore, while Nanosota-9 was only tested therapeutically in mice in this study, its high therapeutic potency suggests it could also be effective as a prophylactic treatment. Overall, combined with our recently developed structure-guided in vitro evolution method, Nanosota-9 has the potential to become a superior anti-Omicron therapeutic.

### Data Availability

The atomic models and corresponding cryo-EM density maps are deposited into the PDB and the Electron Microscopy Data Bank, respectively, with accession numbers PDB 9CO6 and EMDB-45771 (Omicron BA.5 spike complexed with Nanosota-9), PDB 9CO7 and EMDB-45772 (Omicron BA.5 spike complexed with Nanosota-9 after local refinement), PDB 9CO8 and EMDB-45773 (Omicron JN.1 spike complexed with Nanosota-9), and PDB 9CO9 and EMDB-45774 (Omicron JN.1 spike complexed with Nanosota-9 local refinement).

### Methods

### Cell Lines, Plasmids and Viruses

HEK293T cells (American Type Culture Collection (ATCC)) were grown in Dulbecco's modified Eagle medium (DMEM) (containing 10% fetal bovine serum, 2 mM L-glutamine, 100 units/mL penicillin, and 100 μg/mL streptomycin). 293-F cells (ThermoFisher) were grown in FreeStyle 293 Expression Medium (ThermoFisher). Vero E6 cells (ATCC) were cultured in Eagle's minimal essential medium (EMEM) (containing 100 units/ml penicillin, 100 μg/ml streptomycin, and 10% fetal bovine serum). ss320 E. coli (Lucigen) and TG1 E. coli (Lucigen), were grown in 2YT medium. All mammalian cells were authenticated by ATCC using STR profiling and were also tested for mycoplasma contamination. No commonly misidentified cell lines were used.

Mutations were introduced to the original SARS-CoV-2 spike gene to generate the spike genes from the following Omicron subvariants: BA.1 subvariant (GISAID: EPI_ISL_6590782.2), BA.5 subvariant (GISAID: EPI_ISL_12954165), XBB.1.5 subvariant (GISAID: EPI_ISL_17774216), EG.5 subvariant (GISAID: EPI_ISL_17524442), JN.1 subvariant (GISAID: EPI_ISL_17774216), KP.2 subvariant (GISAID: EPI_ISL_19214303), and KP.3 subvariant (GISAID: EPI_ISL_19214243). The spike genes of BA.2.75 (GISAID: EPI_ISL_13502529) and BQ.1 subvariant (GISAID: EPI_ISL_16609492) were synthesized (GenScript). Each of the spike genes was cloned into the pcDNA3.1 (+) vector with a C-terminal C9-tag sequence.

Genes encoding Omicron spike ectodomains (residues 1 to 1208 for BA.1, 1 to 1206 for BA.5, 1 to 1207 for XBB.1.5, and 1 to 1207 for JN.1) were each subcloned into Lenti-CMV vector (Vigene Biosciences) with a C-terminal foldon trimerization tag sequence following a His tag sequence. For the spike ectodomain construct, a D614G mutation, two mutations in the furin cleavage site (from RRAR (SEQ ID NO: 31) to AGAR (SEQ ID NO: 32)), and six proline mutations were introduced to the S2 subunit region to stabilize the spike ectodomains in their prefusion state [32, 33].

A plasmid encoding Fc-tagged Nanosota-9 (Nanosota-9-Fc) was constructed into Lenti-CMV vector with an N-terminal tissue plasminogen activator (tPA) signal peptide sequence and a C-terminal human IgG1 Fc tag sequence.

A gene encoding monomeric Nanosota-9 (Nanosota-9-His) was cloned into PADL22c vector (Antibody Design Labs) with an N-terminal PelB leader sequence and C-terminal His tag and HA tag sequences.

The Omicron viruses (hCoV-19/USA/COR-22-063113/2022 for BA.5, hCoV-19/USA/MD-HP40900/2022 for XBB.1.5, and hCoV-19/USA/New York/PV96109/2023 for JN.1) were obtained through BEI Resources, NIH. Experiments involving live infectious Omicron viruses were conducted at the University of Louisville in approved biosafety level 3 laboratories.

### Construction of Induced Nanobody Phage Display Library

An induced nanobody phage display library was constructed as described previously [30, 34]. Briefly, an alpaca was immunized six times with 125 μg of purified BA.5 spike ectodomain in Gerbu adjuvant. After immunization, blood was collected, and peripheral blood mononuclear cells (PBMCs) were isolated using Sepmate centrifugal devices according to the manufacturer's protocol (Stemcell Technologies). A cDNA library was then constructed through reverse transcription using oligo dT primers and Superscript IV reverse transcriptase (ThermoFisher). A nested PCR strategy was employed to amplify the coding regions of the nanobody fragments. The resulting PCR products were cloned into a modified pADL22 vector (Antibody Design Labs), and the phage library was generated following the manufacturer's protocols (Antibody Design Labs). The final library contained 8×10⁸ colonies. Ten randomly selected clones from the library were sequenced, each showing a unique sequence.

### Screening of Induced Nanobody Phage Display Library

To identify anti-Omicron nanobodies, the above nanobody phage display library was used for bio-panning as previously described [29, 30]. 5 μg of purified BA.5 spike ectodomain in 500 μl of PBS buffer was coated onto an immuno tube (ThermoFisher) and incubated overnight at 4° C. The tube was then blocked with 2% milk in PBS buffer for 2 hours. After blocking, the nanobody phage library was added and incubated for 1 hour with gentle shaking. The tube was washed five times each with PBST (PBS with tween-20) and PBS buffers. After washing, the retained phages were eluted using 500 μl of 100 mM triethylamine, neutralized with 250 μl of 1 M Tris-HCl (pH 7.5), and then used to infect log-phase ss320 E. coli. Single colonies were picked into 96-well plates containing 2YT A+medium and grown overnight at 37° C. The ss320 cultures were then transferred into fresh 2YT medium and grown for 3 hours at 37° C. Nanobody expression was induced with 1 mM IPTG. The supernatants were screened by ELISA against the BA.5 spike ectodomain to identify strong binders. One of the binders, which neutralized BA.5 pseudovirus entry, was named Nanosota-9 and further evaluated.

### Protein Expression and Purification

Nanosota-9-His was expressed and purified from bacteria as previously described [29, 30]. Briefly, the nanobody was extracted from the periplasm of ss320 E. coli following induction with 1 mM IPTG. The E. coli cells were collected, resuspended in 15 ml of TES buffer (0.2 M Tris pH 8, 0.5 mM EDTA, 0.5 M sucrose), and shaken on ice for 1 hour. The suspension was then diluted with 40 ml of ¼ TES buffer and shaken on ice for another hour. The protein in the supernatant was sequentially purified using a Ni-NTA column and a Superdex200 gel filtration column (Cytiva).

Omicron spike ectodomains (with a C-terminal His tag) and Nanosota-9-Fc were produced in 293F mammalian cells as previously described [30, 35]. Briefly, lentiviral particles were generated using a plasmid encoding one of these proteins, which were then used to infect 293F cells for the selection of stable cell lines in the presence of Puromycin (Gibco). Proteins were harvested from the supernatants of the cell culture medium and purified using a Ni-NTA column for His-tagged spike ectodomains or a Protein A column for Nanosota-9-Fc. Further purification was performed using a Superose 6 Increase 10/300 gel filtration column (Cytiva) for spike ectodomains and a Superdex 200 gel filtration column (Cytiva) for Nanosota-9-Fc.

### ELISA

To detect the binding between His-tagged Omicron spike ectodomains and HA-tagged nanobodies, an ELISA was performed as previously described [29, 30]. Briefly, ELISA plates were coated with one of the recombinant Omicron spike ectodomains (100 ng at 2 μg/ml) and incubated overnight at 4° C., followed by blocking with 2% BSA in PBS buffer. After three washes with PBST, the plates were incubated with the supernatant from ss320 E. coli containing one of the nanobodies for 1 hour. After another three washes with PBST buffer, the plates were incubated with an HRP-conjugated anti-HA antibody (1:1,000) (Sigma-Aldrich) for 1 hour. The plates were washed again three times with PBST buffer, and 50 μl of ELISA substrate (Invitrogen) was added. The reactions were stopped by adding 10 μl of 1N H₂SO₄. The absorbance at 450 nm (A450) was measured using a Synergy LX Multi-Mode Reader (BioTek).

To detect the binding between His-tagged Omicron spike ectodomains and Nanosota-9-Fc, an HRP-conjugated anti-human Fc antibody (1:3000) (Jackson ImmunoResearch) was used instead of the anti-HA antibody. All other procedures remained the same as described above.

### Surface Plasmon Resonance (SPR)

To measure the binding affinity between the Omicron spike ectodomains and Nanosota-9-His, a surface plasmon resonance (SPR) assay was conducted using a Biacore S200 system (Cytiva) as previously described [29, 30]. Briefly, each Omicron spike ectodomain was immobilized on a CM5 sensor chip (Cytiva) through chemical crosslinking. Serial dilutions of Nanosota-9-His were injected at different concentrations (8 nM, 16 nM, 32 nM, 64 nM, and 128 nM) using a running buffer composed of 10 mM HEPES, 150 mM NaCl, and 0.05% Tween 20. The resulting data were analyzed using Biacore Evaluation Software (Cytiva).

To assess the potential competition between human ACE2 and Nanosota-9-Fc for binding to the Omicron spike protein, a competition SPR experiment was performed as previously described [30]. The JN.1 spike ectodomain (with a His tag) was immobilized onto two CM5 sensor chips (Cytiva) with 800 resonance units (RU) for each chip. Nanosota-9-Fc (5 μM) was then injected onto the first sensor chip, while running buffer was injected onto the second chip as a control. After saturating the first chip with Nanosota-9-Fc, a mixture of recombinant human ACE2 (His-tagged, at 5 μM) and Nanosota-9-Fc (5 μM) was injected onto the first chip. In the control chip, only ACE2 was injected. The resulting sensorgrams from both chips were overlaid, using the first injection as the baseline. The competitive binding of human ACE2 and Nanosota-9-Fc to the JN.1 spike ectodomain was evaluated by comparing the SPR binding signals from the mixed nanobody/ACE2 injections with those from the ACE2-only injection.

### Flow Cytometry Assay

A flow cytometry assay was performed to evaluate the binding affinity between Nanosota-9-Fc and the spike protein from each Omicron subvariant, as previously described [17, 36]. Briefly, 2 μg of each full-length spike-expressing plasmid with a C-terminal C9 tag was transfected into HEK293T cells in 6-well plates. 24 hours after transfection, cells were detached using Accutase solution (Sigma-Aldrich), washed twice with cold PBS buffer, and resuspended in 500 μl of cold PBS buffer. The cells were fixed with 1 ml of 4% paraformaldehyde (ThermoFisher) and incubated at room temperature for 20 minutes. They were then washed three times with 1% BSA and resuspended in 500 μl of 1% BSA. Nanosota-9-Fc was added to a final concentration of 10 μg/ml and incubated at room temperature for 30 minutes. The cells were washed three times with 1% BSA and resuspended in 200 μl of 1% BSA. Subsequently, 2 μl of anti-C9 antibody and 5 μl of PE anti-human IgG Fc antibody (BioLegend) were added to the cells and incubated on ice in the dark for 30 minutes. The cells were then washed three times with cold PBS buffer and resuspended in 1 ml of cold PBS buffer. Data were acquired using a BD LSRII cytometer and analyzed with FlowJo software (version 10).

### Pseudovirus Entry Assay

The neutralizing potency of Nanosota-9-Fc against Omicron pseudoviruses was assessed using a pseudovirus entry assay as previously described [30, 35]. Briefly, HEK293T cells were co-transfected with a pcDNA3.1(+) plasmid encoding an Omicron spike protein, a helper plasmid psPAX2, and a reporter plasmid plenti-CMV-luc to produce pseudoviruses. The pseudoviruses were harvested 72 hours post-transfection, incubated with nanobodies at varying concentrations at 37° C. for 30 minutes, and then used to transduce HEK293T cells stably expressing human ACE2 in 96-well plates. After 60 hours, the medium was removed, and 80 μl of lysis buffer (Promega) was added to the plates. The cells were lysed with gentle shaking for 25 minutes, and 30 μl of the cell lysate was transferred to 96-well OptiPlates (Revvity Health Sciences). Luciferase substrate (30 μl; Promega) was added to each well, and Relative Light Units (RLUs) were measured using an EnSpire plate reader (PerkinElmer). The efficacy of the nanobody against each Omicron pseudovirus was expressed as the nanobody concentration required to inhibit pseudovirus entry by 50% (IC₅₀).

### SARS-CoV-2 Microneutralization Assay

The neutralizing potency of Nanosota-9-Fc against live Omicron infections was assessed using a virus microneutralization assay as previously described [37]. Briefly, Nanosota-9-Fc was serially diluted tenfold in DMEM, starting at 100 μg/ml. Each dilution was prepared in quadruplicate and mixed with one of the Omicron subvariants-BA.5, XBB.1.5, or JN.1 (0.001 MOI)—at 37° C. for 45 minutes. The mixtures were then added to Vero E6 cells overexpressing ACE2 and TMPRSS2 (A2T2) in a 96-well plate that had been cultured overnight. After 1 hour, the virus/Nanosota-9-Fc mixtures were replaced with 1×DMEM supplemented with 5% FBS. Cell viability was determined after 96 hours of incubation using a Neutral Red assay (Sigma-Aldrich). The efficacy of Nanosota-9-Fc against each Omicron subvariant was expressed as the concentration needed to reduce the virus-induced cytopathic effect (CPE) by 50% (IC₅₀) compared to the control serum-exposed virus.

### Omicron Challenge Experiment in a Mouse Model

The neutralizing potency of Nanosota-9-Fc against infectious Omicron in vivo was assessed using Omicron challenge experiments in a mouse model as previously described [29, 30]. Briefly, female C57BL/6J mice (n=5 per group) were challenged via intranasal inoculation with one of the Omicron subvariants (10+PFU per mouse) in a 50 μl volume of DMEM. Infected mice received either Nanosota-9-Fc (10 mg/kg body weight) or PBS via intraperitoneal injection 4 hours post-challenge. Mice were euthanized at day 2 post-infection, and the collected lung tissues were homogenized and stored at −80° C. until further analysis. Viral titers in the lung tissues were determined by TCID₅₀ assay as previously described [37]. Briefly, Vero E6 cells were seeded into 96-well tissue culture plates and incubated overnight at 37° C. The next day, lung homogenate supernatants were serially diluted tenfold in viral growth medium (DMEM containing 5% FBS) and added in quadruplicates to the cell-seeded plates. The plates were incubated at 37° C. in a humidified incubator with 5% CO2. After 4 days post-infection, cells were fixed in 10% neutral-buffered formalin and stained with 0.1% crystal violet to observe for cytopathic effect (CPE). The TCID₅₀ dose was calculated using the Reed and Muench method.

### Cryo-EM Grid Preparation and Data Acquisition

The complexes of BA.5 spike ectodomains and Nanosota-9-His (4 μl at 0.8 μM) and of JN.1 spike ectodomain and Nanosota-9-His (4 μl at 0.8 μM) were applied to freshly glow-discharged Quantifoil R1.2/1.3 300-mesh copper grids (EM Sciences), blotted for 4 seconds at 22° C. under 100% chamber humidity, and plunge-frozen in liquid ethane using a Vitrobot Mark IV (FEI). Cryo-EM data were collected using EPU (ThermoFisher) equipped with a K3 direct electron detector and a BioQuantum energy filter (Gatan) in CDS mode. Movies were collected at a nominal magnification of 130,000× (corresponding to 0.664 Å per pixel, a slit width of 20 eV, and a nominal defocus value between −1.0 to −2.0 μm. Statistics of cryo-EM data collection is summarized in Table S1.

### Cryo-EM Data Processing, Model Building and Refinement

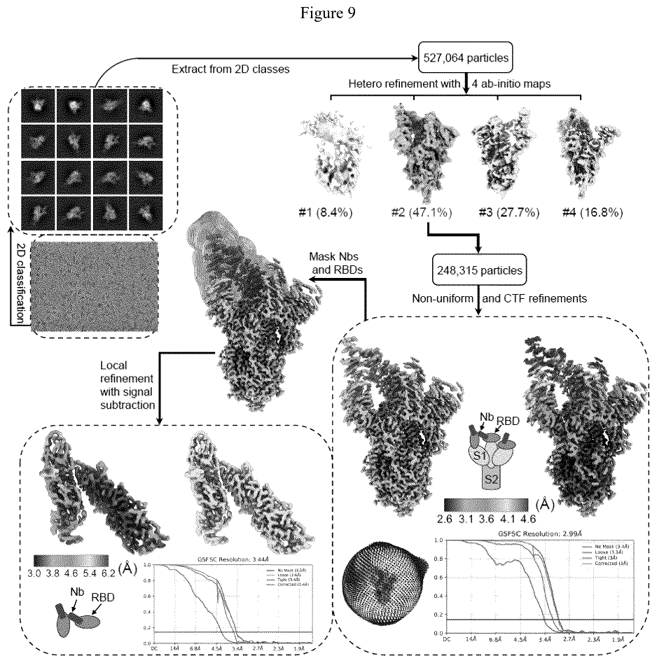

Cryo-EM data were processed using cryoSPARC v4.5.1 [38], with the procedure detailed in FIG. 8 and FIG. 9. Briefly, dose-fractionated movies underwent Patch motion correction with MotionCor2 [39] and Patch CTF estimation with CTFFIND-4.1.13 [40]. Particles were picked using both Blob picker and Template picker in cryoSPARC v4.5.1, and duplicates were removed with the Remove Duplicate Particles Tool. Junk particles were eliminated through multiple rounds of 2D classifications. Particles from the high-quality 2D classes were used for Ab-initio Reconstruction of four maps. These initial models served as starting references for heterogeneous refinement (3D classification). The high-quality 3D classes were further refined using non-uniform, and CTF refinements to generate the final maps. Particles in the good 3D class were imported into RELION-4.0 [41] using the csparc2star.py module (UCSF pyem v0.5. Zenodo) and subjected to signal subtraction to keep only two receptor-binding subunits of the spike and two Nanosota-9 molecules. Particles with the subtracted signal (RBD/Nanosota-9) were then subjected to local refinements to improve densities in cryoSPARC v4.5.1. Map resolutions were determined by gold-standard Fourier shell correlation (FSC) at 0.143 between the two half-maps. Local resolution variation was estimated from the two half-maps in cryoSPARC v4.5.1.

Initial model building of the spike/nanobody complexes was performed in Coot-0.8.9 [42] using PDB 8IOS as the starting models for the BA.5 and JN.1 spike ectodomains, respectively. The initial model of each nanobody was predicted using SWISS-MODEL (swissmodel.expasy.org/) and then fitted into the density map. Several rounds of refinement in Phenix-1.16 [43] and manual building in Coot-0.8.9 were performed until the final reliable models were obtained. Model and map statistics are summarized in Table S1. Figures were generated using UCSF Chimera X v0.93 [44] and PyMol v2.5.2 [45].

TABLE S1
Cryo-EM data collection, refinement and validation
statistics of the spike Nanosota-9 complexes.

| | | | |
|---|---|---|---|
| | Local | | Local |
| | refinement of | | refinement of |
| | BA.5 spike/ | | JN.1 spike/ |
| | Nanosota-9 | | Nanosota-9 |
| | with one RBD | | with one RBD |
| BA.5 spike/ | up and one | JN.1 spike/ | up and one |
| Nanosota-9 | RBD down | Nanosota-9 | RBD down |
| (EMDB-45771) | (EMDB-45772) | (EMDB-45773) | (EMDB-45774) |
| (PDB 9CO6) | (PDB 9CO7) | (PDB 9CO8) | (PDB 9CO9) |

Data collection and processing

| | | | | |
|---|---|---|---|---|
| Magnification | 130,000 | | 130,000 | |
| Voltage (kV) | 300 | | 300 | |
| Electron exposure (e−/Å²) | 54.8 | | 54.8 | |
| Defocus range (μm) | −1.1~−2.0 | | −1.1~−2.0 | |
| Pixel size (Å) | 0.664 | | 0.664 | |
| Symmetry imposed | C1 | | C1 | |
| Initial particle images (no.) | 533,795 | 533,795 | 527,064 | 527,064 |
| Final particle images (no.) | 431,183 | 431,183 | 248,315 | 248,315 |
| Map resolution (Å) | 3.01 | 3.32 | 2.99 | 3.44 |
| FSC threshold | 0.143 | 0.143 | 0.143 | 0.143 |
| Map resolution range (Å) | 2.6-4.6 | 2.9-5.3 | 2.6-4.6 | 3.0-6.2 |

Refinement

| | | | | |
|---|---|---|---|---|
| Initial model used (PDB code) | 8IOS | 8IOS | 8IOS | 8IOS |
| Model resolution (Å) | 3.3 | 3.6 | 3.4 | 3.8 |
| FSC threshold | 0.5 | 0.5 | 0.5 | 0.5 |
| Model resolution range (Å) | 52.3-2.98 | 31.1-1.9 | 45.8-2.6 | 25.2-2.8 |
| Map sharpening B factor (Å²) | −102.1 | −117.5 | −94.2 | −120.2 |

Model composition

| | | | | |
|---|---|---|---|---|
| Non-hydrogen atoms | 28152 | 5222 | 29029 | 5200 |
| Protein residues | 3546 | 666 | 3657 | 661 |
| Ligands | 36 | | 32 | |

B factors (Å²)

| | | | | |
|---|---|---|---|---|
| Protein | 149.64 | 152.92 | 54.19 | 70.26 |
| Nucleotide | | | | |
| Ligand | 134.13 | | 54.33 | |

R.m.s. deviations

| | | | | |
|---|---|---|---|---|
| Bond lengths (Å) | 0.004 | 0.005 | 0.005 | 0.006 |
| Bond angles (°) | 0.832 | 1.076 | 0.985 | 1.021 |

Validation

| | | | | |
|---|---|---|---|---|
| MolProbity score | 1.60 | 1.81 | 1.62 | 1.78 |
| Clashscore | 4.49 | 5.41 | 4.13 | 6.14 |
| Poor rotamers (%) | 0.03 | 0.18 | 0.03 | 0.72 |

Ramachandran plot

| | | | | |
|---|---|---|---|---|
| Favored (%) | 94.43 | 90.88 | 93.61 | 93.11 |
| Allowed (%) | 5.39 | 8.36 | 6.25 | 6.28 |
| Disallowed (%) | 0.17 | 0.76 | 0.14 | 0.61 |

### REFERENCES IN EXAMPLE 1

1. Kaku Y, Yo M S, Tolentino J E, Uriu K, Okumura K, Ito J, et al. Virological characteristics of the SARS-CoV-2 KP.3, LB.1, and KP.2.3 variants. Lancet Infect Dis. 2024. Epub 20240627. doi: 10.1016/s1473-3099 (24) 00415-8. PubMed PMID: 38945150.
2. Karim S S A, Karim Q A. Omicron SARS-CoV-2 variant: a new chapter in the COVID-19 pandemic. Lancet. 2021; 398 (10317): 2126-8. Epub 20211203. doi: 10.1016/s0140-6736 (21) 02758-6. PubMed PMID: 34871545; PubMed Central PMCID: PMCPMC8640673.
3 Klompas M. Understanding Breakthrough Infections Following mRNA SARS-CoV-2 Vaccination. Jama. 2021; 326 (20): 2018-20. doi: 10.1001/jama.2021.19063. PubMed PMID: 34734985.
4. Lipsitch M, Krammer F, Regev-Yochay G, Lustig Y, Balicer R D. SARS-CoV-2 breakthrough infections in vaccinated individuals: measurement, causes and impact. Nature reviews Immunology. 2022; 22 (1): 57-65. Epub 2021 Dec. 9. doi: 10.1038/s41577-021-00662-4. PubMed PMID: 34876702; PubMed Central PMCID: PMCPMC8649989 Bristol Myers Squibb, Janssen, Merck, Sanofi Pasteur, Peter Diamandis/Abundance Platinum, and unpaid advice to One Day Sooner, Pfizer, Janssen, AstraZeneca, Covaxx (United Biomedical), and the Coalition for Epidemic Preparedness Innovations (CEPI). The Icahn School of Medicine at Mount Sinai has filed patent applications relating to SARS-CoV-2 serological assays and NDV-based SARS-CoV-2 vaccines, which list F.K. as co-inventor. Mount Sinai has spun out a company, Kantaro, to market serological tests for SARS-CoV-2. F.K. has consulted for Merck and Pfizer (before 2020) and is currently consulting for Pfizer, Seqirus and Avimex. The Krammer laboratory is also collaborating with Pfizer on animal models of SARS-CoV-2. R.D.B. reports past institutional grants to Clalit Research Institute from Pfizer, outside the submitted work and unrelated to COVID-19, with no direct or indirect personal benefits. G.R.-Y. has received a research grant from Pfizer on an unrelated topic (pneumococcal infections) and has received honoraria from Teva and MSD for presentations given (on fluid contamination and vaccine rollout). Y.L. has received a research grant from Pfizer on an unrelated topic (tick-borne encephalitis).
5. Akinosoglou K, Schinas G, Gogos C. Oral Antiviral Treatment for COVID-19: A Comprehensive Review on Nirmatrelvir/Ritonavir. Viruses. 2022; 14 (11). Epub 20221117. doi: 10.3390/v14112540. PubMed PMID: 36423149; PubMed Central PMCID: PMCPMC9696049.
6. Marzolini C, Kuritzkes D R, Marra F, Boyle A, Gibbons S, Flexner C, et al. Recommendations for the Management of Drug-Drug Interactions Between the COVID-19 Antiviral Nirmatrelvir/Ritonavir (Paxlovid) and Comedications. Clin Pharmacol Ther. 2022; 112 (6): 1191-200. Epub 20220607. doi: 10.1002/cpt.2646. PubMed PMID: 35567754; PubMed Central PMCID: PMCPMC9348462.
7. Duan Y, Zhou H, Liu X, Iketani S, Lin M, Zhang X, et al. Molecular mechanisms of SARS-CoV-2 resistance to nirmatrelvir. Nature. 2023; 622 (7982): 376-82. Epub 20230911. doi: 10.1038/s41586-023-06609-0. PubMed PMID: 37696289.
8. Pantaleo G, Correia B, Fenwick C, Joo V S, Perez L. Antibodies to combat viral infections: development strategies and progress. Nat Rev Drug Discov. 2022; 21 (9): 676-96. Epub 20220620. doi: 10.1038/s41573-022-00495-3. PubMed PMID: 35725925; PubMed Central PMCID: PMCPMC9207876.
9. Du L Y, He Y X, Zhou Y S, Liu S W, Zheng B J, Jiang S B. The spike protein of SARS-CoV—a target for vaccine and therapeutic development. Nature Reviews Microbiology. 2009; 7 (3): 226-36. doi: 10.1038/nrmicro2090. PubMed PMID: ISI: 000263361000014.
10. Li F. Structure, function, and evolution of coronavirus spike proteins. Annual review of virology. 2016; 3:237-61.
11. Shang J, Wan Y, Luo C, Ye G, Geng Q, Auerbach A, et al. Cell entry mechanisms of SARS-CoV-2. Proceedings of the National Academy of Sciences. 2020; 117 (21): 11727-34.
12. Shang J, Ye G, Shi K, Wan Y, Luo C, Aihara H, et al. Structural basis of receptor recognition by SARS-CoV-2. Nature. 2020; 581 (7807): 221-4.
13. Li F. Receptor recognition mechanisms of coronaviruses: a decade of structural studies. Journal of virology. 2015; 89 (4): 1954-64.
14. Piccoli L, Park Y J, Tortorici M A, Czudnochowski N, Walls A C, Beltramello M, et al. Mapping Neutralizing and Immunodominant Sites on the SARS-CoV-2 Spike Receptor-Binding Domain by Structure-Guided High-Resolution Serology. Cell. 2020; 183 (4): 1024-42.e21. Epub 20200916. doi: 10.1016/j.cell.2020.09.037. PubMed PMID: 32991844; PubMed Central PMCID: PMCPMC7494283.
15. Harvey W T, Carabelli A M, Jackson B, Gupta R K, Thomson E C, Harrison E M, et al. SARS-CoV-2 variants, spike mutations and immune escape. Nat Rev Microbiol. 2021; 19 (7): 409-24. Epub 2021 Jun. 3. doi: 10.1038/s41579-021-00573-0. PubMed PMID: 34075212; PubMed Central PMCID: PMCPMC8167834.
16. Gobeil S M, Janowska K, McDowell S, Mansouri K, Parks R, Stalls V, et al. Effect of natural mutations of SARS-CoV-2 on spike structure, conformation, and antigenicity. Science. 2021; 373 (6555). Epub 2021 Jun. 26. doi: 10.1126/science.abi6226. PubMed PMID: 34168071; PubMed Central PMCID: PMCPMC8611377.
17. Geng Q, Wan Y, Hsueh F-C, Shang J, Ye G, Bu F, et al. Lys417 acts as a molecular switch that regulates the conformation of SARS-CoV-2 spike protein. eLife. 2023; 12: e74060.
18. Miller N L, Raman R, Clark T, Sasisekharan R. Complexity of Viral Epitope Surfaces as Evasive Targets for Vaccines and Therapeutic Antibodies. Frontiers in immunology. 2022; 13:904609. Epub 20220617. doi: 10.3389/fimmu.2022.904609. PubMed PMID: 35784339; PubMed Central PMCID: PMCPMC9247215.
19. Könning D, Zielonka S, Grzeschik J, Empting M, Valldorf B, Krah S, et al. Camelid and shark single domain antibodies: structural features and therapeutic potential. Curr Opin Struct Biol. 2017; 45:10-6. Epub 2016 Nov. 20. doi: 10.1016/j.sbi.2016.10.019. PubMed PMID: 27865111.
20. De Meyer T, Muyldermans S, Depicker A. Nanobody-based products as research and diagnostic tools. Trends Biotechnol. 2014; 32 (5): 263-70. Epub 2014 Apr. 5. doi: 10.1016/j.tibtech.2014.03.001. PubMed PMID: 24698358.
21. Yang Y, Li F, Du L. Therapeutic nanobodies against SARS-CoV-2 and other pathogenic human coronaviruses. J Nanobiotechnology. 2024; 22 (1): 304. Epub 20240531. doi: 10.1186/s12951-024-02573-7. PubMed PMID: 38822339; PubMed Central PMCID: PMCPMC11140877.
22. Nambulli S, Xiang Y, Tilston-Lunel N L, Rennick L J, Sang Z, Klimstra W B, et al. Inhalable Nanobody (PiN-21) prevents and treats SARS-CoV-2 infections in Syrian hamsters at ultra-low doses. Science advances. 2021; 7 (22). Epub 2021 May 28. doi: 10.1126/sciadv.abh0319. PubMed PMID: 34039613; PubMed Central PMCID: PMCPMC8153718.
23. Wu X, Cheng L, Fu M, Huang B, Zhu L, Xu S, et al. A potent bispecific nanobody protects hACE2 mice against SARS-CoV-2 infection via intranasal administration. Cell reports. 2021; 37 (3): 109869. Epub 2021 Oct. 14. doi: 10.1016/j.celrep.2021.109869. PubMed PMID: 34644535; PubMed Central PMCID: PMCPMC8492916.
24 Muyldermans S. Nanobodies: natural single-domain antibodies. Annu Rev Biochem. 2013; 82:775-97. Epub 2013 Mar. 19. doi: 10.1146/annurev-biochem-063011-092449. PubMed PMID: 23495938.
25. Steeland S, Vandenbroucke R E, Libert C. Nanobodies as therapeutics: big opportunities for small antibodies. Drug discovery today. 2016; 21 (7): 1076-113. Epub 2016 Apr. 16. doi: 10.1016/j.drudis.2016.04.003. PubMed PMID: 27080147.
26. Scully M, Cataland S R, Peyvandi F, Coppo P, Knöbl P, Kremer Hovinga J A, et al. Caplacizumab Treatment for Acquired Thrombotic Thrombocytopeniarpura. N Engl J Med. 2019; 380 (4): 335-46. Epub 2019 Jan. 10. doi: 10.1056/NEJMoa1806311. PubMed PMID: 30625070.
27. He L, Wu Q, Zhang Z, Chen L, Yu K, Li L, et al. Development of Broad-Spectrum Nanobodies for the Therapy and Diagnosis of SARS-CoV-2 and Its Multiple Variants. Mol Pharm. 2024. Epub 20240626. doi: 10.1021/acs.molpharmaceut.4c00165. PubMed PMID: 38920116.
28. Ye G, Bu F, Pan R, Mendoza A, Saxena D, Zheng J, et al. Dual-role epitope on SARS-CoV-2 spike enhances and neutralizes viral entry across different variants. PLOS Pathog. 2024; 20 (9): e1012493. Epub 20240905. doi: 10.1371/journal.ppat.1012493. PubMed PMID: 39236072; PubMed Central PMCID: PMCPMC11407660.
29. Ye G, Gallant J, Zheng J, Massey C, Shi K, Tai W, et al. The development of Nanosota-1 as anti-SARS-CoV-2 nanobody drug candidates. eLife. 2021; 10. Epub 2021 Aug. 3. doi: 10.7554/eLife.64815. PubMed PMID: 34338634; PubMed Central PMCID: PMCPMC8354634.
30. Ye G, Pan R, Bu F, Zheng J, Mendoza A, Wen W, et al. Discovery of Nanosota-2, -3, and -4 as super potent and broad-spectrum therapeutic nanobody candidates against COVID-19. Journal of Virology. 2023: e01448-23.
31. Ye G, Bu F, Pan R, Mendoza A, Yang G, Spiller B, et al. Structure-guided in vitro evolution of nanobodies targeting new viral variants. PLOS Pathog. 2024; 20 (9): e1012600. Epub 20240926. doi: 10.1371/journal.ppat. 1012600. PubMed PMID: 39325826; PubMed Central PMCID: PMCPMC11460708.
32. Hsieh C L, Goldsmith J A, Schaub J M, DiVenere A M, Kuo H C, Javanmardi K, et al. Structure-based design of prefusion-stabilized SARS-CoV-2 spikes. Science. 2020; 369 (6510): 1501-5. Epub 2020 Jul. 25. doi: 10.1126/science.abd0826. PubMed PMID: 32703906; PubMed Central PMCID: PMCPMC7402631.
33. Ye G, Liu B, Li F. Cryo-EM structure of a SARS-CoV-2 omicron spike protein ectodomain. Nature communications. 2022; 13 (1): 1214. Epub 2022 Mar. 5. doi: 10.1038/s41467-022-28882-9. PubMed PMID: 35241675; PubMed Central PMCID: PMCPMC8894419.
34. Pardon E, Laeremans T, Triest S, Rasmussen S G, Wohlkönig A, Ruf A, et al. A general protocol for the generation of Nanobodies for structural biology. Nature protocols. 2014; 9 (3): 674-93. Epub 2014 Mar. 1. doi: 10.1038/nprot.2014.039. PubMed PMID: 24577359; PubMed Central PMCID: PMCPMC4297639.
35. Geng Q, Tai W, Baxter V K, Shi J, Wan Y, Zhang X, et al. Novel virus-like nanoparticle vaccine effectively protects animal model from SARS-CoV-2 infection. PLOS Pathog. 2021; 17 (9): e1009897. Epub 2021 Sep. 8. doi: 10.1371/journal.ppat. 1009897. PubMed PMID: 34492082; PubMed Central PMCID: PMCPMC8448314 following competing interests: The University of Minnesota has filed a patent on the lumazine synthase nanoparticle-based SARS-CoV-2 RBD vaccine with F.L, Q.G., Y.W., J.S., M.J., S.H., L.D. and T.W. as inventors. Other authors have declared that no competing interests exist.
36. Hsueh F C, Shi K, Mendoza A, Bu F, Zhang W, Aihara H, et al. Structural basis for raccoon dog receptor recognition by SARS-CoV-2. PLOS Pathog. 2024; 20 (5):e1012204. Epub 20240506. doi: 10.1371/journal.ppat. 1012204. PubMed PMID: 38709834; PubMed Central PMCID: PMCPMC11098500.
37. Amanat F, White K M, Miorin L, Strohmeier S, McMahon M, Meade P, et al. An In Vitro Microneutralization Assay for SARS-CoV-2 Serology and Drug Screening. Curr Protoc Microbiol. 2020; 58 (1):e108. doi: 10.1002/cpmc.108. PubMed PMID: 32585083; PubMed Central PMCID: PMCPMC7361222.
38. Punjani A, Rubinstein J L, Fleet D J, Brubaker M A. cryoSPARC: algorithms for rapid unsupervised cryo-EM structure determination. Nature methods. 2017; 14 (3): 290-6. Epub 2017 Feb. 7. doi: 10.1038/nmeth.4169. PubMed PMID: 28165473.
39. Rubinstein J L, Brubaker M A. Alignment of cryo-EM movies of individual particles by optimization of image translations. Journal of structural biology. 2015; 192 (2): 188-95. Epub 2015 Aug. 25. doi: 10.1016/j.jsb.2015.08.007. PubMed PMID: 26296328.
40. Rohou A, Grigorieff N. CTFFIND4: Fast and accurate defocus estimation from electron micrographs. Journal of structural biology. 2015; 192 (2): 216-21. Epub 2015 Aug. 19. doi: 10.1016/j.jsb.2015.08.008. PubMed PMID: 26278980; PubMed Central PMCID: PMCPMC6760662.
41. Zivanov J, Nakane T, Scheres S H W. Estimation of high-order aberrations and anisotropic magnification from cryo-EM data sets in RELION-3.1. IUCrJ. 2020; 7 (Pt 2): 253-67. Epub 2020 Mar. 10. doi: 10.1107/s2052252520000081. PubMed PMID: 32148853; PubMed Central PMCID: PMCPMC7055373.
42. Emsley P, Cowtan K. Coot: model-building tools for molecular graphics. Acta Crystallographica Section D-Biological Crystallography. 2004; 60:2126-32. doi: 10.1107/s0907444904019158. PubMed PMID: WOS: 000225360500002.
43. Adams P D, Afonine P V, Bunkoczi G, Chen V B, Davis I W, Echols N, et al. PHENIX: a comprehensive Python-based system for macromolecular structure solution. Acta Crystallographica Section D-Biological Crystallography. 2010; 66:213-21. doi: 10.1107/s0907444909052925. PubMed PMID: ISI: 000273820800013.
44. Goddard T D, Huang C C, Meng E C, Pettersen E F, Couch G S, Morris J H, et al. UCSF ChimeraX: Meeting modern challenges in visualization and analysis. Protein science: a publication of the Protein Society. 2018; 27 (1): 14-25. Epub 2017 Jul. 16. doi: 10.1002/pro.3235. PubMed PMID: 28710774; PubMed Central PMCID: PMCPMC5734306.
45. DeLano W L. PyMOL: an open-source molecular graphics tool. CCP4 Newsl Protein Crystallogr. 2002; 40:82-92.

### EXAMPLE 2. STRUCTURE-GUIDED NANOBODY EVOLUTION AGAINST SARS-CoV-2 ESCAPE

SARS-CoV-2 continues to evade existing antibody therapeutics, forcing repeated, resource-intensive discovery cycles. A structure-guided, in vitro evolution approach was employed herein that rapidly retargets a validated nanobody to the Q493E escape mutation in the spike receptor-binding domain (RBD). Rather than restarting discovery for each variant, we reuse the existing nanobody (Nanosota-9A) and structural insights to produce next-generation inhibitors (e.g., Nanosota-9B) via a streamlined engineering workflow. This strategy offers a practical path to maintain therapeutic coverage as the virus evolves, conserving research resources and shortening response times. Beyond SARS-CoV-2, the platform applies to other rapidly mutating viruses in which viral escape mutations undermine antibody therapeutics, providing a template for durable, updatable antiviral therapeutics.

### INTRODUCTION

The COVID-19 pandemic exposed a core vulnerability of antibody-based antivirals: once escape variants emerge, therapies can rapidly lose efficacy and be withdrawn, wasting prior investment and delaying care while new binders are rediscovered and redeveloped [1-3]. This outcome is predictable-during widespread transmission, mutations accumulate in the viral surface glycoproteins that mediate host-cell entry, undermining practical usage of antibody drugs [4-6]. Yet antibodies retain key advantages over small molecules: they bind viral glycoproteins with high specificity, have favorable safety profiles, and possess long in vivo half-lives that often enable single-dose regimens [7, 8]. Preserving these strengths while preventing viral escape will be important in future pandemics. Here in this Example 2, we pursue that goal through structure-guided evolution of nanobodies.

Nanobodies are single-domain antibodies derived from camelid heavy-chain-only antibodies [9, 10]. Their compact, modular architecture confers several antiviral advantages over conventional antibodies: access to cryptic epitopes and favorable tissue penetration; high in vitro stability that reduces cold-chain dependence; compatibility with intranasal or pulmonary delivery; and a straightforward phage-display workflow [11-17]. We recently developed a structure-guided strategy to rapidly adapt nanobodies to viral escape variants [18]. This approach maps escape mutations at the nanobody-antigen interface, builds focused libraries by randomizing nanobody residues proximal to those changes, and selects binders to the mutant antigen via phage display. The resulting targeted evolution proceeds far faster, while achieving comparable effectiveness to natural immune adaptation. It exploits the engineering simplicity of nanobodies' single-domain scaffolds, an advantage not matched by the two-chain architecture of conventional antibodies. As proof of concept, we restored binding and neutralization against two prior mutations in the SARS-CoV-2 spike protein [18], which mediates viral entry [19, 20]. In this Example 2, we apply this strategy to counter a newly emerged, clinically relevant spike escape mutation.

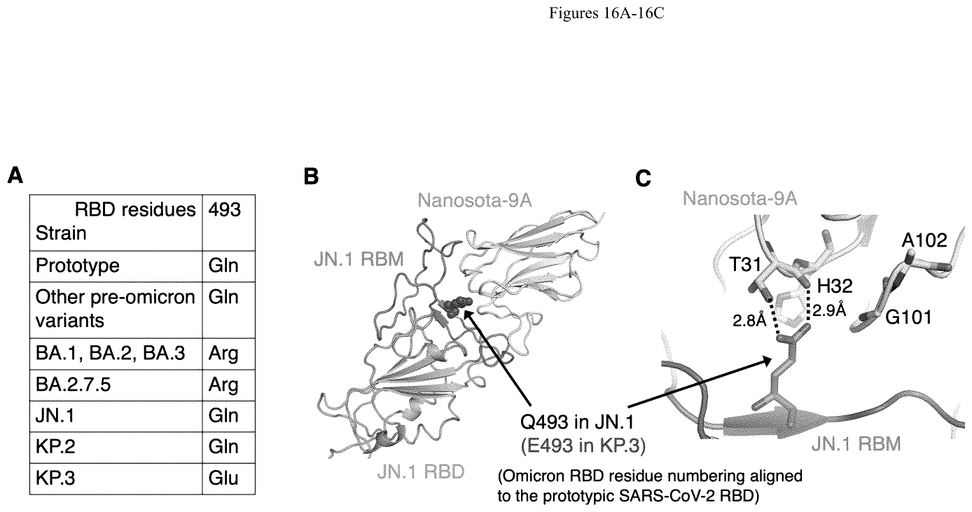

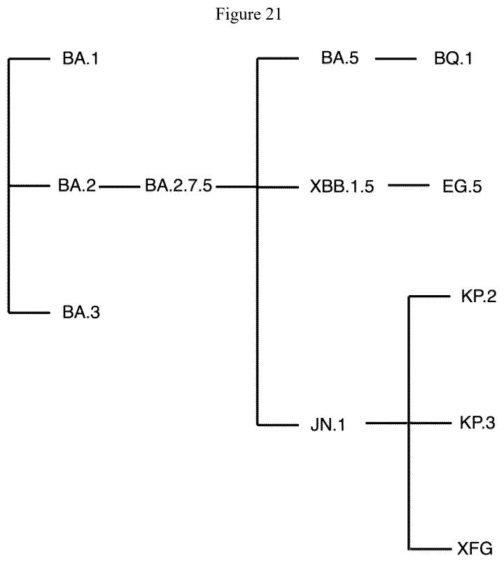

The Omicron variant of SARS-CoV-2 emerged in late 2021 and rapidly displaced previous variants worldwide due to increased transmissibility (FIG. 21) [21, 22]. The earliest Omicron subvariants, BA.1, BA.2, and BA.3, were first detected in quick succession; BA.2 became dominant as BA.1 and BA.3 waned. BA.2 has continued to diversify, and recent subvariants (e.g., KP.2, KP.3, XFG) descend from JN.1, a BA.2 derivative [23, 24]. During this evolution, the spike accumulated numerous mutations, many of which conferred antibody escape [25, 26]. A recent mutation, Q493E in the receptor-binding domain (RBD) of KP.2, KP.3, and XFG spikes, is particularly concerning (FIG. 16A). In prototypic SARS-CoV-2, Gln493 forms two strong hydrogen bonds with Lys31 and Glu35 of the human ACE2 receptor [27, 28]. Early Omicron subvariants replaced this residue with Arg493, a change incompatible with human Lys31 but compatible with Asn31 in mouse ACE2, consistent with a rodent origin hypothesis for Omicron [29]. Later subvariants, including JN.1 reverted to Gln493, restoring affinity for human ACE2 [30]. Glu493 in KP.2/KP.3/XFG RBDs introduces charge repulsion with human Glu35, lowering the affinity of the interaction between the RBD and ACE2 and aligning with immune escape [26]. Because many RBD-directed antibodies target this region, the Q493E mutation is clinically significant [25, 26].

To date, we have developed nine nanobodies—the Nanosota series-targeting the SARS-CoV-2 spike [14, 31-33]. Nanosota-9 binds an RBD epitope that includes residue 493. It potently neutralizes JN.1 by directly blocking RBD-ACE2 binding but loses activity against KP.3 because of the Q493E mutation (FIGS. 16B, 16C) [32]. Here, we use our structure-guided in vitro evolution platform to overcome the Q493 mutation. More broadly, we propose an adaptable antiviral paradigm “update, don't rediscover”: build nanobody arsenals against key SARS-CoV-2 escape mutations, beginning with the engineering of Nanosota-9 to counter the Q493E mutation in the Omicron spike.

### Results

### Design and Application of a Structure-Guided In Vitro Evolution Strategy for Nanosota-9

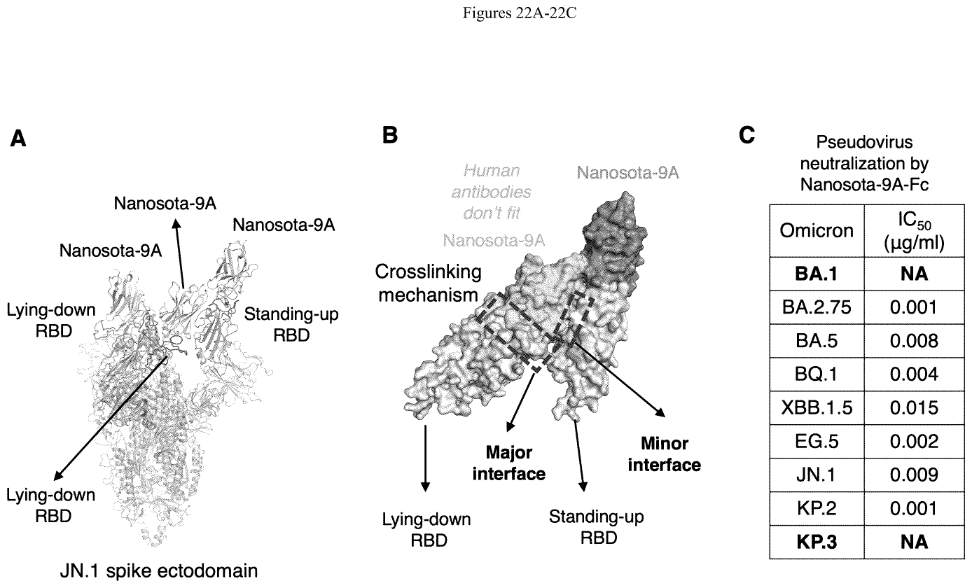

We previously determined the cryo-EM structure of the JN.1 spike ectodomain complexed with Nanosota-9 [32]. The structure shows two Nanosota-9 molecules cross-linking two RBDs within the trimeric spike-one RBD in the “standing-up” (ACE2-accessible) state and the other RBD in the “lying-down” (immune-evasive) state (FIG. 22A, 22B). The RBD contains two subdomains: a core and the receptor-binding motif (RBM), which mediates ACE2 engagement [19, 27, 34]. One Nanosota-9 binds the RBM of the standing-up RBD and directly blocks ACE2 binding. The other binds the core of the lying-down RBD, stabilizes it in the lying-down conformation, and indirectly blocks ACE2 binding. This cross-linking mechanism underlies the high neutralization potency of Nanosota-9 against Omicron. Notably, the RBM epitope targeted by Nanosota-9 is relatively conserved compared with other RBD epitopes because of its essential role in ACE2 binding, which helps explain Nanosota-9's broad anti-Omicron activity: Nanosota-9 effectively neutralizes most Omicron subvariants, including JN.1 and KP.2 (FIG. 22C). However, it fails to neutralize KP.3 due to a single Q493E mutation in the RBD (Q489 in JN.1; E485 in KP.3) [32]. For consistency, we refer to this site as residue 493 (as in prototypic SARS-CoV-2) throughout, despite numbering differences among Omicron subvariants arising from spike insertions/deletions. This Example 2 aimed to engineer Nanosota-9 to overcome the Q493E mutation in the KP.3 RBD.

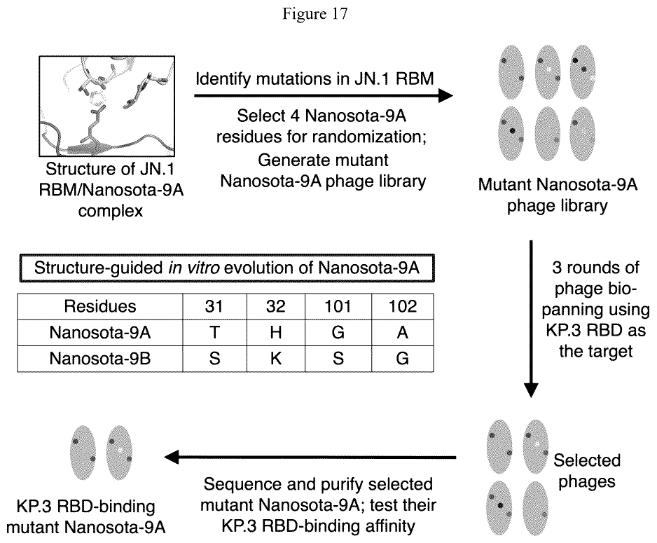

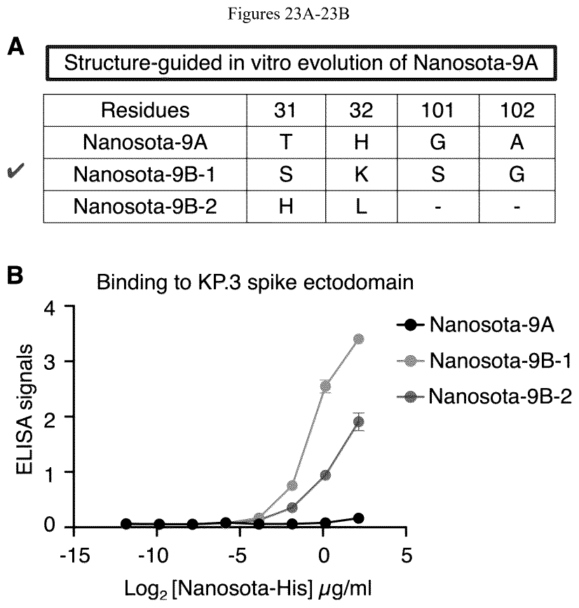

One strategy was to apply the structure-based in vitro evolution approach to Nanosota-9 (FIG. 17). At the JN.1 RBD/Nanosota-9 interface, Gln493 of the JN.1 RBD forms two hydrogen bonds with Thr31 of Nanosota-9-one with its side chain and the other with its main-chain carbonyl oxygen (FIG. 16B, 16C). In the KP.3 RBD, residue 493 is a Glu, which would be incompatible with the main-chain carbonyl oxygen of Thr31 in Nanosota-9. Therefore, to overcome this mutation, we needed to modify the local structure of Nanosota-9 around this site to accommodate Glu493 in the KP.3 RBD. To this end, we selected four residues near the mutation, Thr31/His32/Gly 101/Ala102, and completely randomized them simultaneously. These randomized changes were introduced into the gene encoding Nanosota-9, now termed Nanosota-9A, in the phage vector using PCR primers. The mutant Nanosota-9A phage display library was then constructed and screened with the KP.3 RBD as the bait. Phages that bound to the KP.3 RBD were sequenced to identify the mutant nanobody genes they carried. The results revealed two mutant nanobodies with enhanced affinity for the KP.3 RBD: one containing Ser31/Lys32/Ser101/Gly 102, and the other His31/Leu32/deletion101/deletion102 (FIG. 23A). Because the former showed higher binding affinity for the KP.3 RBD based on ELISA (FIG. 23B), it was selected for further characterization and designated Nanosota-9B.

### Functional and Structural Characterizations of Nanosota-9B

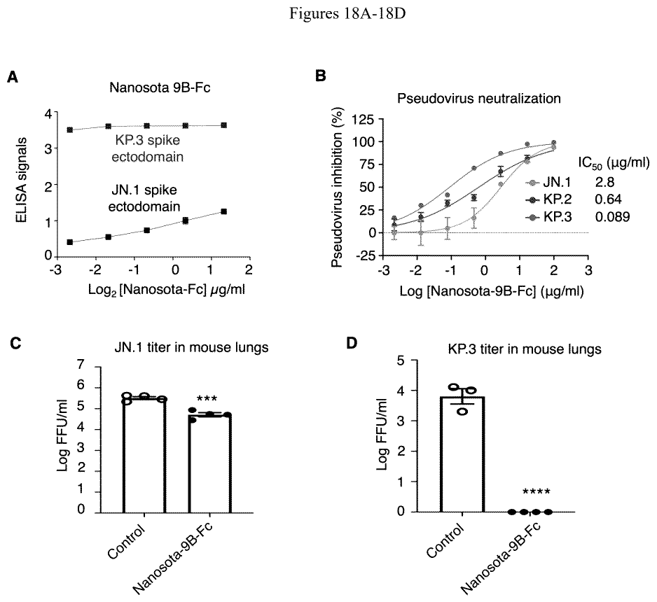

We evaluated the anti-Omicron activities of Nanosota-9B, focusing on JN.1 and KP.3. First, we measured the affinity of Nanosota-9B for Omicron spike ectodomains using ELISA. The results showed that human Fc-tagged Nanosota-9B (Nanosota-9B-Fc) binds the KP.3 spike significantly more strongly than the JN.1 spike (FIG. 18A). This contrasts with Nanosota-9A-Fc, which binds the JN.1 spike strongly but does not bind the KP.3 spike [32]. Second, we assessed the potency of Nanosota-9B in neutralizing Omicron spike-mediated entry. We performed an Omicron pseudovirus entry assay in which HIV pseudotyped with an Omicron spike infected ACE2-expressing human cells in the presence of Nanosota-9B-Fc. The results showed that Nanosota-9B-Fc neutralized KP.3 pseudoviruses significantly more efficiently than JN.1 pseudoviruses, with IC₅₀ values of 0.089 μg/mL and 2.8 μg/mL, respectively (FIG. 18B). It also neutralized KP.2 pseudoviruses with an IC₅₀ of 0.64 μg/mL. This also contrasts with Nanosota-9A, which potently neutralizes JN.1 pseudoviruses but does not neutralize KP.3 pseudoviruses [32]. Together, these data demonstrate that Nanosota-9B targets KP.3 significantly more efficiently than JN.1, while retaining meaningful activity against JN.1. In contrast, Nanosota-9A potently targets JN.1 but does not target KP.3.

We further evaluated the anti-Omicron potency of Nanosota-9B in mice. We used the Fc-tagged form (Nanosota-9B-Fc) for several reasons. Fc tagging typically enhances antiviral potency by increasing valency and potentially inducing antibody-dependent cellular cytotoxicity (ADCC). In addition, with a molecular weight of ˜75 kDa, Fc-tagged nanobodies have prolonged in vivo half-life (typically 5-10 days) because they exceed the kidney filtration threshold of ˜60 kDa. Despite the added mass, Fc-tagged nanobodies are still about half the size of conventional antibodies, remain compatible with intranasal delivery, and retain high in vitro stability. Importantly, they preserve the single-domain antigen-binding architecture. For in vivo efficacy testing, C57BL/6 mice were challenged with either JN.1 or KP.3. Four hours after infection, Nanosota-9B-Fc was administered by intraperitoneal (i.p.) injection. Lung tissues were collected 2 days post-infection, and viral titers were measured. Nanosota-9B-Fc reduced JN.1 titers in the lungs by 6-fold, and reduced KP.3 titers to undetectable levels (FIGS. 18C, 18D). Consistent with the in vitro data, these in vivo results show that Nanosota-9B-Fc provides complete protection against KP.3 infection and only modest protection against JN.1.

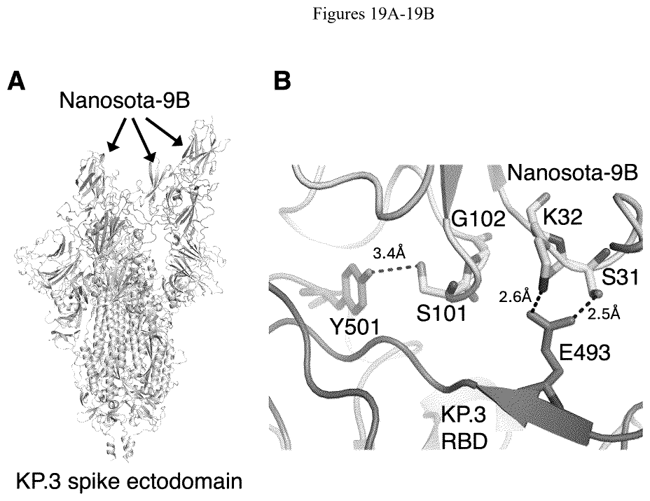

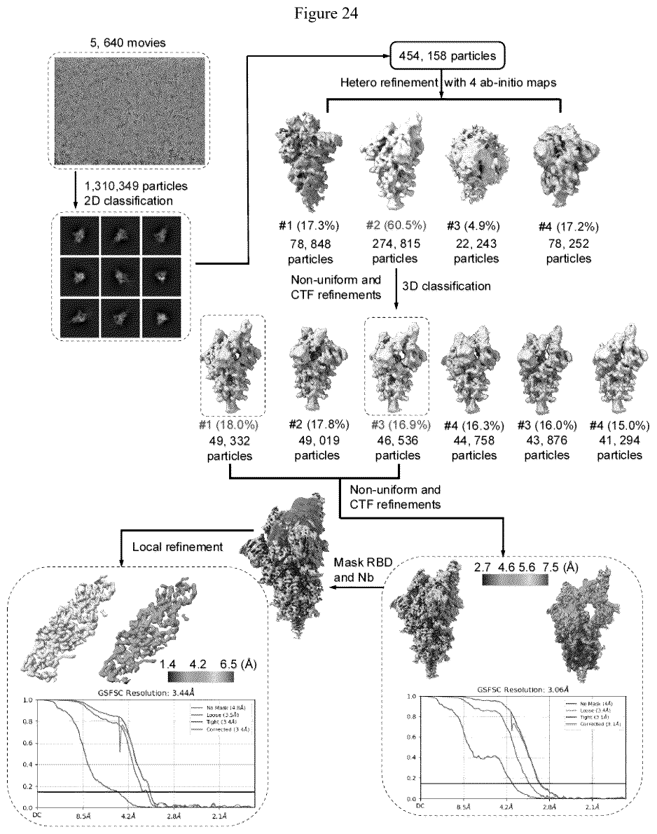

To elucidate the structural basis for Nanosota-9B's high-affinity binding to the KP.3 spike, we determined the cryo-EM structure of the KP.3 spike ectodomain complexed with Nanosota-9B (FIG. 24; Table S2). Nanosota-9B engages the trimeric KP.3 spike in the same manner as Nanosota-9A engages the trimeric JN.1 spike: two Nanosota-9B molecules crosslink two RBDs, with one RBD in the standing-up state and the other in the lying-down state (FIG. 19A). The new structure reveals a modified interaction network at the interface between the KP.3 RBD and Nanosota-9B, arising from substitutions in both the RBD and the nanobody (FIG. 19B). Specifically, Glu493 in the KP.3 RBD forms a hydrogen bond with Ser31 and a salt bridge with Lys32 of Nanosota-9B. The main-chain carbonyl of Thr31 in Nanosota-9A, which previously conflicted with Glu493, is now reoriented and no longer clashes with this residue. Although the additional substitutions in Nanosota-9B at residues 101 and 102 do not directly accommodate Glu493, Ser101 forms a hydrogen bond with Tyr501 in the KP.3 RBD, further strengthening the interface. These structural analyses define the molecular mechanism by which Nanosota-9B was engineered to bind the KP.3 RBD with high affinity.

### Construction and Characterization of Bispecific Nanobody Nanosota-9A/9B-Fc

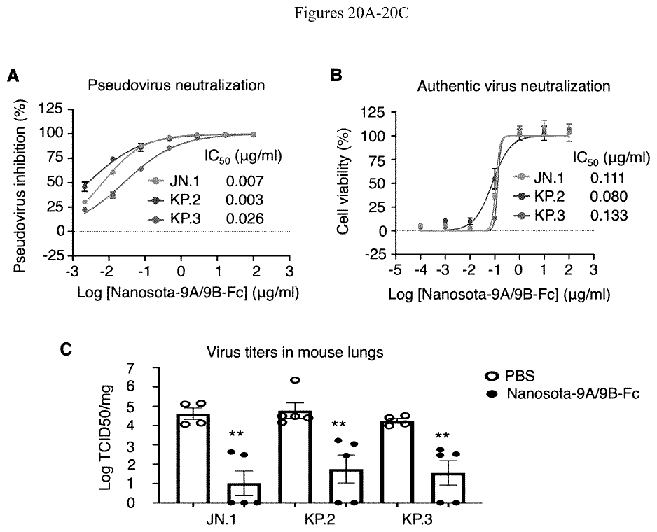

In Example 1 we developed Nanosota-9A, which potently inhibits JN.1 but not KP.3, and in Example 2 we developed Nanosota-9B, which potently inhibits KP.3 but shows only moderate activity against JN.1. To create a single inhibitor effective against both Omicron JN.1 and KP.3 subvariants, we constructed the bispecific nanobody Nanosota-9A/9B-Fc by fusing Nanosota-9A and Nanosota-9B to a human Fc domain. In vitro, Nanosota-9A/9B-Fc potently neutralized JN.1, KP.2, and KP.3 pseudoviruses with IC₅₀ values of 0.007, 0.003, and 0.026 μg/mL (FIG. 20A), respectively, and it also neutralized the corresponding authentic viruses with IC₅₀ values of 0.111, 0.080, and 0.133 μg/mL (FIG. 20B), respectively. For in vivo testing, C57BL/6 mice were challenged with JN.1, KP.2, or KP.3, treated intraperitoneally with Nanosota-9A/9B-Fc 4 hours after infection, and assessed for lung viral titers 2 days later; the bispecific nanobody significantly reduced titers for all three subvariants by approximately 10³ to 10⁵ fold (FIG. 20C). These results show that Nanosota-9A/9B-Fc is a potent inhibitor of JN.1, KP.2, and KP.3 in vitro and in vivo.

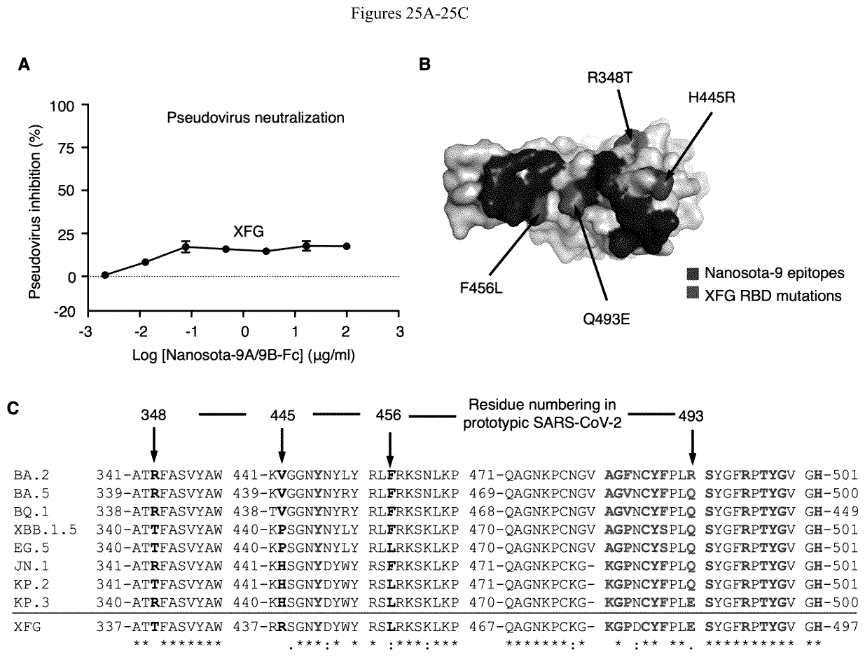

We next tested the neutralizing potency of Nanosota-9A/9B-Fc against XFG, the dominant Omicron subvariant in the United States (85% as of September 2025) [35], but it did not neutralize XFG pseudoviruses (FIG. 25A). Sequence alignment of Omicron RBDs identified four substitutions between JN.1 and XFG in or near the Nanosota-9 epitope: R348T and H445R (proximal to the epitope) and F456L and Q493E (within the epitope) (FIGS. 25B, 25C). Because Nanosota-9B accommodates the Q493E mutation and Nanosota-9A accommodates the F456L mutation, which is also present in KP.2 and KP.3, the loss of Nanosota-9A/9B-Fc activity against XFG is likely attributable to the R348T and H445R mutations. Addressing these two mutations will be the focus of future work, whereas the Example 2 expands our Nanosota arsenal by overcoming the Q493E mutation and validates our “update, do not rediscover” antiviral paradigm.

### DISCUSSION

Rapid escape mutations in respiratory RNA viruses such as SARS-CoV-2 expose a core limitation of conventional antibody therapeutics: an antibody's paratope (its antigen-binding site) can fail abruptly when a single escape mutation on the viral target abolishes binding, forcing slow, repeated, and resource-intensive discovery cycles. A durable antiviral platform must therefore either target conserved viral epitopes or enable swift, rational adaptation to viral-variant-specific mutations. In practice, the most potent SARS-CoV-2 antibodies target the spike RBD, whereas antibodies targeting non-RBD epitopes generally lack sufficient antiviral potency to advance to clinical use [36-38]. Therapies must therefore confront the evolutionarily dynamic RBD directly. We propose an “update, don't rediscover” paradigm for nanobody therapeutics, using structure-guided in vitro nanobody evolution to counter key viral escape mutations and to continually expand antiviral arsenals. Nanobodies are well suited to this approach because their single-domain architecture simplifies molecular cloning and bacterial expression in phage display workflows, whereas conventional two-chain antibodies complicate them. Nanobodies therefore offer a practical route to rapid, durable responses to current and future viral variants.

We apply this blueprint to a concrete Omicron RBD problem to demonstrate its practical relevance. Starting from a validated Nanosota-9A scaffold that is potent against JN.1 but impaired by the Q493E mutation in the KP.3 RBD, we mapped the mutation-induced clash at the RBD-nanobody interface and fully randomized four nanobody residues proximal to the mutation site. Selection against the KP.3 RBD yielded Nanosota-9B, which overcomes the Q493E mutation by locally reorganizing around Glu493 and forming new stabilizing contacts with the KP.3 RBD. Functionally, Nanosota-9B binds and neutralizes KP.3 more strongly than JN.1 while retaining moderate JN.1 activity. A bispecific nanobody combining Nanosota-9A and Nanosota-9B potently neutralizes JN.1, KP.2, and KP.3 in vitro and in vivo. Because protein-protein interfaces are intrinsically plastic [39, 40], multiple engineering solutions can defeat a given viral escape mutation, depending on which proximal nanobody residues are randomized. In previous work, for example, we re-engineered Nanosota-3A, active against BA.1 but not XBB.1.5, into Nanosota-3C, which targets both BA.1 and XBB.1.5 RBDs with high affinity, showing that adaptation to a new viral variant can preserve activity against the prior one [18]. Thus, for each recurrent viral escape mutation, multiple viral-variant-adapted nanobodies and the design rules behind them can be accumulated across epitopes on the SARS-CoV-2 spike, building coverage against a broad set of mutations. Starting from the basic case as shown in the current Example, in which one nanobody addresses a single escape mutation within one epitope, this framework further generalizes in two ways: (i) adapting a single nanobody to multiple escape mutations within the same epitope and (ii) extending to panels of nanobodies that target distinct epitopes on the SARS-CoV-2 spike.

A central advantage of this “update, don't rediscover” strategy is cumulative reuse. Each successful adaptation contributes a reusable module: a viral-variant-adapted paratope, its structural rationale, and a validated set of residues for future randomization that can be redeployed or recombined as related mutations recur. This maximizes prior investment and speeds subsequent updates. By contrast, rediscovery cycles for conventional antibodies rarely carry antiviral efforts forward; newly selected antibodies replace rather than augment earlier antibodies and offer limited reusability when new viral variants emerge.

In sum, updating rather than rediscovering offers a practical way to keep pace in the arms race with viruses. It provides a rapid, mechanistically grounded workflow; preserves and builds on previous work through modular arsenals of viral-variant-adapted nanobodies; and yields binders whose activity tracks newly dominant viral mutations. Although viral escape mutations are frequent, there are finite evolutionary pathways available to a virus. As surveillance highlights recurrent viral mutation hotspots, new designs of nanobodies can begin immediately using the accumulated knowledge base. This nanobody-centered, structure-guided updating provides a potential path to sustain antiviral coverage while avoiding the delays and wastefulness inherent to rediscovery.

### Data Availability

The atomic models and corresponding cryo-EM density maps have been deposited into the PDB and the Electron Microscopy Data Bank, respectively, with accession numbers 9Z1M and EMDB-73749 (KP.3 spike complexed with Nanosota-9B), and 9Z1N and EMDB-73750 (KP.3 spike complexed with Nanosota-9B; after local refinement).

### Methods

### Cell Lines, Plasmids and Viruses

HEK293T and Vero E6-ACE2-TMPRSS2 cells (ATCC) were cultured in Dulbecco's modified Eagle medium (DMEM) supplemented with 10% fetal bovine serum. TG1 and SS320 E. coli (Lucigen) were grown in 2YT medium. The prototypic SARS-CoV-2 spike gene (GenBank: QHD43416.1) was synthesized (GenScript) with the D614G substitution. The XFG spike gene (GISAID: EPI_ISL_20063407) was also synthesized (Twist Bioscience). Mutations were introduced into the prototypic spike gene to generate spike genes corresponding to the Omicron subvariants JN.1 (GISAID: EPI_ISL_17774216), KP.2 (GISAID: EPI_ISL_19214303), and KP.3 (GISAID: EPI_ISL_19214243). All spike genes were cloned into the pcDNA3.1(+) vector.

Genes encoding SARS-CoV-2 spike ectodomains (residues 1-1207 for JN.1 and 1-1203 for KP.3) were subcloned into the pCAGGS vector (Addgene) with a C-terminal His tag and foldon trimerization sequence. In these constructs, the furin cleavage motif RRAR (SEQ ID NO: 31) was replaced with AGAR (SEQ ID NO: 32), and six proline substitutions were introduced into the S2 subunit as described previously [41, 42]. Genes encoding Fc-tagged nanobodies were cloned into pCAGGS with an N-terminal tPA signal peptide and a C-terminal human IgG1 Fc (GenBank: AEV43323.1). To generate the bispecific nanobody Nanosota-9A/9B-Fc, a “knobs-into-holes” strategy was used, introducing T366Y and Y407T into the Fc regions of the pCAGGS-Nanosota-9A and pCAGGS-Nanosota-9B constructs, respectively [43].

SARS-CoV-2 isolates hCoV-19/USA/New York/PV96109/2023 (subvariant JN.1; BEI NR-59693), hCoV-19/USA/CA-GBW-GKISBBBB26982/2024 (subvariant KP.2; BEI NR-59890), and hCoV-19/USA/NJ-GBW-GKISBBBB88291/2024 (subvariant KP 3; BEI NR-59892) were obtained from BEI Resources, NIAID, NIH.

### Structure-Guided In Vitro Evolution of Nanosota-9

To enhance binding to the KP.3 RBD, Nanosota-9A was subjected to structure-guided in vitro evolution [32]. Random mutations at Thr31, His32, Gly 101, and Ala102 were introduced by PCR, generating a library of Nanosota-9A variants. The mutant nanobody genes were cloned into the PADL22c vector (Antibody Design Labs) and introduced into TG1 cells by electroporation to construct a mutagenic phage-display library. Phages with improved binding to the KP.3 spike ectodomain were then selected by bio-panning. After two rounds, the best-binding phages were identified, their nanobody genes sequenced, and the corresponding nanobodies tested for affinity to the KP.3 spike ectodomain by ELISA. The top binder, named Nanosota-9B, contains four mutations: T31S, H32K, G101S, and A102G.

### Protein Expression and Purification

Nanosota-9A and Nanosota-9B were expressed and purified as previously described [44]. Briefly, His- and HA-tagged nanobodies were purified from the periplasm of ss320 E. coli after induction with 1 mM IPTG. E. coli cells were harvested and resuspended in 15 mL TES buffer (0.2 M Tris, pH 8.0; 0.5 mM EDTA; 0.5 M sucrose). Proteins in the supernatant were purified sequentially using a Ni-NTA column and a Superdex 200 gel-filtration column (Cytiva).

The SARS-CoV-2 spike ectodomains (JN.1 and KP.3, each His-tagged) and Fc-tagged nanobodies were prepared as previously described [32]. Plasmids were transiently transfected into Expi293F cells using polyethylenimine (PEI; Polysciences). Supernatants were harvested three days post-transfection. Proteins were purified using a Ni-NTA affinity column (Cytiva), followed by a Superose 6 gel-filtration column (Cytiva) for spike ectodomains or a Superdex 200 gel-filtration column (Cytiva) for Fc-tagged nanobodies.

### ELISA

ELISA was performed to measure the binding affinity between His-tagged SARS-CoV-2 spike ectodomains and HA-tagged nanobodies as previously described [44]. Briefly, ELISA plates were coated with a recombinant SARS-CoV-2 spike ectodomain and then sequentially incubated with nanobodies (either nanobodies from ss320 E. coli supernatant or recombinant nanobodies) and HRP-conjugated anti-HA antibody (1:2,000; Sigma). The ELISA substrate (Invitrogen) was added, the reaction was stopped with 1 N H₂SO₄, and absorbance at 450 nm was measured on a Synergy LX Multi-Mode Reader (BioTek).

ELISA was also performed to detect binding between His-tagged spike ectodomains and Nanosota-9B-Fc, following the procedure above, except that an HRP-conjugated anti-human Fc antibody (1:3,000; Jackson ImmunoResearch) was used instead of the HRP-conjugated anti-HA antibody.

### Pseudovirus Entry Assay

Pseudovirus entry assays were performed as previously described [32]. Pseudoviruses were generated by co-transfecting HEK293T cells with a pcDNA3.1(+) plasmid encoding a full-length spike, the helper plasmid psPAX2 (lentiviral backbone), and the luciferase reporter plasmid pLenti-CMV-luc. After 72 hours, pseudoviruses were collected, incubated with nanobodies at varying concentrations at 37° C. for 1 hour, and then used to infect HEK293T cells expressing human ACE2. After an additional 60 hours, cells were lysed. Aliquots of the lysates were transferred to new plates, luciferase substrate was added, and relative light units (RLUs) were measured on an EnSpire plate reader (PerkinElmer). Nanobody efficacy was reported as the IC₅₀, the concentration required to inhibit pseudovirus entry by 50%.

### Omicron Microneutralization Assay

The neutralizing potency of Nanosota-9A/9B-Fc against authentic Omicron infection was assessed by virus microneutralization as previously described [45]. Briefly, Nanosota-9A/9B-Fc was tenfold serially diluted in DMEM starting at 100 μg/mL. Each dilution (quadruplicate) was mixed with one Omicron subvariant (JN.1, KP.2, or KP.3) at an MOI of 0.01 and incubated at 37° C. for 45 min. Mixtures were added to Vero E6 cells overexpressing human ACE2 and human TMPRSS2 (A2T2) seeded the previous day in 96-well plates. After 1 h, the inoculum was replaced with 1×DMEM containing 5% FBS. Cell viability was measured after 96 h using a Neutral Red assay (Sigma-Aldrich). Potency was reported as the IC₅₀—the concentration required to reduce virus-induced cytopathic effect by 50% relative to virus-only controls.

### Evaluation of the Anti-Omicron Potency of Nanosota-9B-Fc in Mice

C57BL/6 male mice (10 weeks old) were obtained from Charles River Laboratories. They were housed in groups of up to five, given free access to food and water, and kept on a 12-hour light/dark cycle. Mice were lightly anesthetized with ketamine/xylazine and intranasally challenged with 10⁵ PFU of the Omicron subvariant JN.1 or KP.3. Four hours after infection, mice received 10 mg/kg Nanosota-9B-Fc intraperitoneally. Clinical scores and body weight were monitored daily. All mice were euthanized on day 2 post-infection, and lungs were collected into DMEM and homogenized.

Virus titers were measured by focus-forming assay (FFA). Lung homogenate supernatants were diluted tenfold in DMEM; 50 μL of each dilution was added to confluent VeroE6-ACE2-TMPRSS2 cells in flat-bottom 96-well plates and incubated for 45 minutes at 37° C. with 5% CO2. The inoculum was replaced with 100 μL of overlay medium (10% FCS, penicillin/streptomycin, 1.2% carboxymethylcellulose). Plates were incubated for 20 hours at 37° C., 5% CO2. After fixation, cells were washed with 0.1% Tween-20/PBS, then permeabilized and blocked for 30 minutes at room temperature in 0.1% Triton X-100/1% BSA/PBS. Viral foci were stained with a mouse anti-SARS-CoV/SARS-CoV-2 nucleocapsid antibody (SinoBiological; 1:1,000 in PBS with 1% BSA), followed by an HRP-conjugated goat anti-mouse IgG (Invitrogen; 1:500 in PBS with 1% BSA) for 1 hour at 37° C. Bound antibodies were detected with KPL TrueBlue Peroxidase Substrate (SeraCare) for 10 minutes at room temperature. Plates were rinsed with distilled water, air-dried, and imaged; foci were counted using a CTL ImmunoSpot Analyzer.

### Evaluation of the Anti-Omicron Potency of Nanosota-9A/9B-Fc in Mice

The neutralizing potency of Nanosota-9A/9B-Fc against authentic Omicron in vivo was assessed in mice as previously described [32]. Briefly, female C57BL/6J mice (n=5 per group) were challenged by intranasal inoculation with Omicron subvariants JN.1, KP.2, or KP.3 (10\hat{ }4 PFU per mouse) in 50 μL DMEM. Infected mice received either Nanosota-9A/9B-Fc (10 mg/kg body weight) or PBS by intraperitoneal injection 4 hours post-challenge. Mice were euthanized on day 2 post-infection; lungs were collected, homogenized, and stored at −80° C. until analysis.

Viral titers in lung tissue were determined by TCID₅₀ as previously described [45]. Vero E6 cells were seeded in 96-well tissue-culture plates and incubated overnight at 37° C. The next day, lung-homogenate supernatants were tenfold serially diluted in growth medium (DMEM with 5% FBS) and added in quadruplicate to the plates. Cultures were incubated at 37° C. in 5% CO2. At 4 days post-infection, cells were fixed in 10% neutral-buffered formalin and stained with 0.1% crystal violet to assess cytopathic effect. TCID₅₀ values were calculated using the Reed-Muench method.

### Cryo-EM Grid Preparation and Data Acquisition

KP.3 spike ectodomain was mixed with a 1.5-fold molar excess of Nanosota-9B for 1 h before grid preparation. Four microliters (0.8 mg/mL) of the mixture were applied to freshly glow-discharged Quantifoil R1.2/1.3, 300-mesh copper grids (Electron Microscopy Sciences), blotted for 4 s at 22° C. under 100% chamber humidity, and plunge-frozen in liquid ethane using a Vitrobot Mark IV (FEI). Cryo-EM data were collected in EPU (Thermo Fisher Scientific) with a K3 direct electron detector and a BioContinuum energy filter (Gatan) at a nominal magnification of 130,000× (0.664 Å/pixel) (Table S1).

### Cryo-EM Image Processing

Cryo-EM data were processed in cryoSPARC v4.5.1 [46]. Dose-fractionated movies underwent Patch motion correction with MotionCor2 [47] and Patch CTF estimation with CTFFIND [48]. Particles were picked with the Blob Picker and Template Picker, followed by the Remove Duplicate Particles tool. Junk particles were discarded through three rounds of 2D classification. Particles from the good 2D classes were used for ab initio reconstruction of four maps, then subjected to 3D classification into six classes. The two best 3D classes were refined further with non-uniform and CTF refinements to generate the final maps. To improve density at the RBD-nanobody interface, local refinements focused on one lying-down RBD (the best-resolved of the three) and the Nanosota-9B region. Map resolutions were determined by gold-standard Fourier shell correlation (FSC) at 0.143 between the two half-maps. Local resolution was estimated from the half-maps in cryoSPARC v4.5.1 (FIG. 24).

### Cryo-EM Model Building and Refinement.

Initial model building of the spike-nanobody complexes was performed in Coot v0.8.9 [49] using PDB 9CO8 as the starting model [32]. Models were refined through iterative cycles of Phenix v1.16 [50] and manual rebuilding in Coot until final, reliable models were obtained. Standing-up RBDs and their bound nanobodies are generally flexible; therefore, they were fitted into the density as rigid bodies. In the local map of the KP.3 spike/Nanosota-9B complex, an atomic model was built for the interface between the lying-down RBD and Nanosota-9B (Table S2). Figures were generated in PyMOL (The PyMOL Molecular Graphics System, v3.0; Schrödinger, LLC).

TABLE S2
Cryo-EM data collection, refinement and validation statistics
of the KP.3 spike ectodomain/Nanosota-9B complex.

| | |
|---|---|
| | KP.3 spike |
| KP.3 spike | ectodomain/ |
| ectodomain/ | Nanosota-9B |
| Nanosota-9B | complex after |
| complex | local refinement |

Data collection and processing

| | | |
|---|---|---|
| Magnification | 130,000 | 130,000 |
| Voltage (kV) | 300 | 300 |
| Electron exposure (e−/Å²) | 51.2 | 50 |
| Defocus range (μm) | −0.75~−2.5 | −0.75~−2.5 |
| Pixel size (Å) | 0.664 | 0.664 |
| Symmetry imposed | C1 | C1 |
| Initial particle images (no.) | 1,310,349 | 1,310,349 |
| Final particle images (no.) | 95,868 | 95,868 |
| Map resolution (Å) | 3.06 | 3.44 |
| FSC threshold | 0.143 | 0.143 |
| Map resolution range (Å) | 1.4-6.5 | 2.5-7.8 |

Refinement

| | | |
|---|---|---|
| Initial model used (PDB code) | 9CO8 | 9CO8 |
| Model resolution (Å) | 3.3 | 3.7 |
| FSC threshold | 0.5 | 0.5 |
| Model resolution range (Å) | 3.0-57.1 | 1.8-22.3 |
| Map sharpening B factor (Å²) | 69.3 | 97.5 |

Model composition

| | | |
|---|---|---|
| Non-hydrogen atoms | 28753 | 3098 |
| Protein residues | 3633 | 395 |
| Ligands | 26 | 0 |

B factors (Å²)

| | | |
|---|---|---|
| Protein | 144.34 | 96.36 |
| Nucleotide | | |
| Ligand | 145.41 | |

R.m.s. deviations

| | | |
|---|---|---|
| Bond lengths (Å) | 0.005 | 0.005 |
| Bond angles (°) | 0.912 | 0.926 |

Validation

| | | |
|---|---|---|
| MolProbity score | 1.56 | 1.42 |
| Clashscore | 3.55 | 2.31 |
| Poor rotamers (%) | 0.03 | 0.00 |

Ramachandran plot

| | | |
|---|---|---|
| Favored (%) | 93.77 | 93.86 |
| Allowed (%) | 5.99 | 6.14 |
| Disallowed (%) | 0.25 | 0.00 |

### REFERENCES IN EXAMPLE 2

1. Flemming A. Omicron, the great escape artist. Nature reviews Immunology. 2022; 22 (2): 75. doi: 10.1038/s41577-022-00676-6. PubMed PMID: 35017722; PubMed Central PMCID: PMCPMC8749340.
2. Planas D, Saunders N, Maes P, Guivel-Benhassine F, Planchais C, Buchrieser J, et al. Considerable escape of SARS-CoV-2 Omicron to antibody neutralization. Nature. 2022; 602 (7898): 671-5. Epub 20211223. doi: 10.1038/s41586-021-04389-z. PubMed PMID: 35016199.
3. Chew K W, Moser C, Daar E S, Wohl D A, Li J Z, Coombs R, et al. Bamlanivimab reduces nasopharyngeal SARS-CoV-2 RNA levels but not symptom duration in non-hospitalized adults with COVID-19: A Phase 2 Randomized Clinical Trial. medRxiv. 2021. Epub 20211221. doi: 10.1101/2021.12.17.21268009. PubMed PMID: 34981077; PubMed Central PMCID: PMCPMC8722620.
4. Greaney A J, Starr T N, Gilchuk P, Zost S J, Binshtein E, Loes A N, et al. Complete Mapping of Mutations to the SARS-CoV-2 Spike Receptor-Binding Domain that Escape Antibody Recognition. Cell host & microbe. 2021; 29 (1): 44-57.e9. Epub 20201119. doi: 10.1016/j.chom.2020.11.007. PubMed PMID: 33259788; PubMed Central PMCID: PMCPMC7676316.
5. Greaney A J, Loes A N, Crawford K H D, Starr T N, Malone K D, Chu H Y, et al. Comprehensive mapping of mutations in the SARS-CoV-2 receptor-binding domain that affect recognition by polyclonal human plasma antibodies. Cell host & microbe. 2021; 29 (3): 463-76.e6. Epub 20210208. doi: 10.1016/j.chom.2021.02.003. PubMed PMID: 33592168; PubMed Central PMCID: PMCPMC7869748.
6. Ye G, Liu B, Li F. Cryo-EM structure of a SARS-CoV-2 omicron spike protein ectodomain. Nature communications. 2022; 13 (1): 1214.
7. Ryman J T, Meibohm B. Pharmacokinetics of Monoclonal Antibodies. CPT Pharmacometrics Syst Pharmacol. 2017; 6 (9): 576-88. Epub 20170729. doi: 10.1002/psp4.12224. PubMed PMID: 28653357; PubMed Central PMCID: PMCPMC5613179.
8. Opacic M, Lin K. Tutorial on Monoclonal Antibody Pharmacokinetics and Its Considerations in Early Development. Clin Transl Sci. 2018; 11 (6): 540-52. Epub 20180807. doi: 10.1111/cts. 12567. PubMed PMID: 29877608; PubMed Central PMCID: PMCPMC6226118.
9. Könning D, Zielonka S, Grzeschik J, Empting M, Valldorf B, Krah S, et al. Camelid and shark single domain antibodies: structural features and therapeutic potential. Curr Opin Struct Biol. 2017; 45:10-6. Epub 2016 Nov. 20. doi: 10.1016/j.sbi.2016.10.019. PubMed PMID: 27865111.
10. De Meyer T, Muyldermans S, Depicker A. Nanobody-based products as research and diagnostic tools. Trends Biotechnol. 2014; 32 (5): 263-70. Epub 2014 Apr. 5. doi: 10.1016/j.tibtech.2014.03.001. PubMed PMID: 24698358.
11. Muyldermans S. Nanobodies: natural single-domain antibodies. Annu Rev Biochem. 2013; 82:775-97. Epub 2013 Mar. 19. doi: 10.1146/annurev-biochem-063011-092449. PubMed PMID: 23495938.
12. Steeland S, Vandenbroucke R E, Libert C. Nanobodies as therapeutics: big opportunities for small antibodies. Drug discovery today. 2016; 21 (7): 1076-113. Epub 2016 Apr. 16. doi: 10.1016/j.drudis.2016.04.003. PubMed PMID: 27080147.
13. Romao E, Morales-Yanez F, Hu Y, Crauwels M, De Pauw P, Hassanzadeh G G, et al. Identification of Useful Nanobodies by Phage Display of Immune Single Domain Libraries Derived from Camelid Heavy Chain Antibodies. Current pharmaceutical design. 2016; 22 (43): 6500-18. Epub 2016 Sep. 28. doi: 10.2174/1381612822666160923114417. PubMed PMID: 27669966.
14. Ye G, Gallant J, Zheng J, Massey C, Shi K, Tai W, et al. The development of Nanosota-1 as anti-SARS-CoV-2 nanobody drug candidates. eLife. 2021; 10. Epub 2021 Aug. 3. doi: 10.7554/eLife.64815. PubMed PMID: 34338634; PubMed Central PMCID: PMCPMC8354634.
15. Wu X, Cheng L, Fu M, Huang B, Zhu L, Xu S, et al. A potent bispecific nanobody protects hACE2 mice against SARS-CoV-2 infection via intranasal administration. Cell reports. 2021; 37 (3): 109869. Epub 2021 Oct. 14. doi: 10.1016/j.celrep.2021.109869. PubMed PMID: 34644535; PubMed Central PMCID: PMCPMC8492916.
16. Nambulli S, Xiang Y, Tilston-Lunel N L, Rennick L J, Sang Z, Klimstra W B, et al. Inhalable Nanobody (PiN-21) prevents and treats SARS-CoV-2 infections in Syrian hamsters at ultra-low doses. Science advances. 2021; 7 (22). Epub 2021 May 28. doi: 10.1126/sciadv.abh0319. PubMed PMID: 34039613; PubMed Central PMCID: PMCPMC8153718.
17. Schoof M, Faust B, Saunders R A, Sangwan S, Rezelj V, Hoppe N, et al. An ultrapotent synthetic nanobody neutralizes SARS-CoV-2 by stabilizing inactive Spike. Science. 2020; 370 (6523): 1473-9. Epub 2020 Nov. 7. doi: 10.1126/science.abe3255. PubMed PMID: 33154106; PubMed Central PMCID: PMCPMC7857409.
18. Ye G, Bu F, Pan R, Mendoza A, Yang G, Spiller B, et al. Structure-guided in vitro evolution of nanobodies targeting new viral variants. PLOS Pathog. 2024; 20 (9): e1012600.
19. Li F. Structure, function, and evolution of coronavirus spike proteins. Annual review of virology. 2016; 3:237-61.
20. Shang J, Wan Y, Luo C, Ye G, Geng Q, Auerbach A, et al. Cell entry mechanisms of SARS-CoV-2. Proceedings of the National Academy of Sciences. 2020; 117 (21): 11727-34.
21. Geng Q, Shi K, Ye G, Zhang W, Aihara H, Li F. Structural Basis for Human Receptor Recognition by SARS-CoV-2 Omicron Variant BA.1. Journal of Virology. 2022; 96 (8): e00249-22.
22. Bálint G, Vörös-Horváth B, Széchenyi A. Omicron: increased transmissibility and decreased pathogenicity. Signal transduction and targeted therapy. 2022; 7 (1): 151. Epub 20220507. doi: 10.1038/s41392-022-01009-8. PubMed PMID: 35525870; PubMed Central PMCID: PMCPMC9077027.
23. Planas D, Staropoli I, Michel V, Lemoine F, Donati F, Prot M, et al. Distinct evolution of SARS-CoV-2 Omicron XBB and BA.2.86/JN.1 lineages combining increased fitness and antibody evasion. Nature communications. 2024; 15 (1): 2254. Epub 20240313. doi: 10.1038/s41467-024-46490-7. PubMed PMID: 38480689; PubMed Central PMCID: PMCPMC10938001.
24. Lu Y, Ao D, He X, Wei X. The rising SARS-CoV-2 JN.1 variant: evolution, infectivity, immune escape, and response strategies. MedComm (2020). 2024; 5 (8): e675. Epub 20240729. doi: 10.1002/mco2.675. PubMed PMID: 39081516; PubMed Central PMCID: PMCPMC11286544.
25. Planas D, Staropoli I, Planchais C, Yab E, Jeyarajah B, Rahou Y, et al. Escape of SARS-CoV-2 Variants KP.1.1, LB.1, and KP3.3 From Approved Monoclonal Antibodies. Pathog Immun. 2024; 10 (1): 1-11. Epub 20240930. doi: 10.20411/pai.v10i1.752. PubMed PMID: 39391808; PubMed Central PMCID: PMCPMC11464000.
26. Wang Q, Mellis I A, Ho J, Bowen A, Kowalski-Dobson T, Valdez R, et al. Recurrent SARS-CoV-2 spike mutations confer growth advantages to select JN.1 sublineages. Emerg Microbes Infect. 2024; 13 (1): 2402880. Epub 20240916. doi: 10.1080/22221751.2024.2402880. PubMed PMID: 39259045; PubMed Central PMCID: PMCPMC11407393.
27. Shang J, Ye G, Shi K, Wan Y, Luo C, Aihara H, et al. Structural basis of receptor recognition by SARS-CoV-2. Nature. 2020; 581 (7807): 221-4.
28. Wan Y, Shang J, Graham R, Baric R S, Li F. Receptor recognition by the novel coronavirus from Wuhan: an analysis based on decade-long structural studies of SARS coronavirus. Journal of virology. 2020; 94 (7): 10.1128/jvi.00127-20.
29. Zhang W, Shi K, Geng Q, Ye G, Aihara H, Li F. Structural basis for mouse receptor recognition by SARS-CoV-2 omicron variant. Proceedings of the National Academy of Sciences. 2022; 119 (44): e2206509119.
30. Zhang W, Shi K, Geng Q, Herbst M, Wang M, Huang L, et al. Structural evolution of SARS-CoV-2 omicron in human receptor recognition. Journal of Virology. 2023; 97 (8): e00822-23.
31. Ye G, Bu F, Pan R, Mendoza A, Saxena D, Zheng J, et al. Dual-role epitope on SARS-CoV-2 spike enhances and neutralizes viral entry across different variants. PLOS Pathog. 2024; 20 (9): e1012493. Epub 20240905. doi: 10.1371/journal.ppat. 1012493. PubMed PMID: 39236072; PubMed Central PMCID: PMCPMC11407660.
32. Ye G, Bu F, Saxena D, Turner-Hubbard H, Herbst M, Spiller B, et al. Discovery of Nanosota-9 as anti-Omicron nanobody therapeutic candidate. PLOS Pathog. 2024; 20 (11): e1012726. Epub 20241126. doi: 10.1371/journal.ppat. 1012726. PubMed PMID: 39591462.
33. Ye G, Pan R, Bu F, Zheng J, Mendoza A, Wen W, et al. Discovery of Nanosota-2, -3, and -4 as super potent and broad-spectrum therapeutic nanobody candidates against COVID-19. Journal of Virology. 2023: e01448-23.
34. Li F. Receptor recognition mechanisms of coronaviruses: a decade of structural studies. Journal of virology. 2015; 89 (4): 1954-64.
35. CDC. www.cdc.gov/covid/php/variants/variants-and-genomic-surveillance.html. 2025.
36. Piccoli L, Park Y J, Tortorici M A, Czudnochowski N, Walls A C, Beltramello M, et al. Mapping Neutralizing and Immunodominant Sites on the SARS-CoV-2 Spike Receptor-Binding Domain by Structure-Guided High-Resolution Serology. Cell. 2020; 183 (4): 1024-42.e21. Epub 20200916. doi: 10.1016/j.cell.2020.09.037. PubMed PMID: 32991844; PubMed Central PMCID: PMCPMC7494283.
37. Pinto D, Sauer M M, Czudnochowski N, Low J S, Tortorici M A, Housley M P, et al. Broad betacoronavirus neutralization by a stem helix-specific human antibody. Science. 2021; 373 (6559): 1109-16. Epub 20210806. doi: 10.1126/science.abj3321. PubMed PMID: 34344823; PubMed Central PMCID: PMCPMC9268357.
38. Barnes C O, Jette C A, Abernathy M E, Dam K A, Esswein S R, Gristick H B, et al. SARS-CoV-2 neutralizing antibody structures inform therapeutic strategies. Nature. 2020; 588 (7839): 682-7. Epub 20201012. doi: 10.1038/s41586-020-2852-1. PubMed PMID: 33045718; PubMed Central PMCID: PMCPMC8092461.
39. Atwell S, Ultsch M, De Vos A M, Wells J A. Structural plasticity in a remodeled protein-protein interface. Science. 1997; 278 (5340): 1125-8. doi: 10.1126/science.278.5340.1125. PubMed PMID: 9353194.
40. Matthews J M. Plasticity at Protein-Protein Interaction Interfaces. In: Roberts GCK, editor. Encyclopedia of Biophysics. Berlin, Heidelberg: Springer Berlin Heidelberg; 2013. p. 1886-8.
41. Hsieh C L, Goldsmith J A, Schaub J M, DiVenere A M, Kuo H C, Javanmardi K, et al. Structure-based design of prefusion-stabilized SARS-CoV-2 spikes. Science. 2020; 369 (6510): 1501-5. Epub 2020 Jul. 25. doi: 10.1126/science.abd0826. PubMed PMID: 32703906; PubMed Central PMCID: PMCPMC7402631.
42. Geng Q, Wan Y, Hsueh F-C, Shang J, Ye G, Bu F, et al. Lys417 acts as a molecular switch that regulates the conformation of SARS-CoV-2 spike protein. eLife. 2023; 12: e74060.
43. Ridgway J B, Presta L G, Carter P. ‘Knobs-into-holes’ engineering of antibody CH3 domains for heavy chain heterodimerization. Protein Eng. 1996; 9 (7): 617-21. doi: 10.1093/protein/9.7.617. PubMed PMID: 8844834.
44. Ye G, Gallant J, Zheng J, Massey C, Shi K, Tai W, et al. The development of Nanosota-1 as anti-SARS-CoV-2 nanobody drug candidates. eLife. 2021; 10: e64815.
45. Amanat F, White K M, Miorin L, Strohmeier S, McMahon M, Meade P, et al. An In Vitro Microneutralization Assay for SARS-CoV-2 Serology and Drug Screening. Curr Protoc Microbiol. 2020; 58 (1):e108. doi: 10.1002/cpmc.108. PubMed PMID: 32585083; PubMed Central PMCID: PMCPMC7361222.
46. Punjani A, Rubinstein J L, Fleet D J, Brubaker M A. cryoSPARC: algorithms for rapid unsupervised cryo-EM structure determination. Nature methods. 2017; 14 (3): 290-6. Epub 2017 Feb. 7. doi: 10.1038/nmeth.4169. PubMed PMID: 28165473.
47. Rubinstein J L, Brubaker M A. Alignment of cryo-EM movies of individual particles by optimization of image translations. Journal of structural biology. 2015; 192 (2): 188-95. Epub 2015 Aug. 25. doi: 10.1016/j.jsb.2015.08.007. PubMed PMID: 26296328.
48. Rohou A, Grigorieff N. CTFFIND4: Fast and accurate defocus estimation from electron micrographs. Journal of structural biology. 2015; 192 (2): 216-21. Epub 2015 Aug. 19. doi: 10.1016/j.jsb.2015.08.008. PubMed PMID: 26278980; PubMed Central PMCID: PMCPMC6760662.
49. Emsley P, Cowtan K. Coot: model-building tools for molecular graphics. Acta Crystallographica Section D-Biological Crystallography. 2004; 60:2126-32. doi: 10.1107/s0907444904019158. PubMed PMID: WOS: 000225360500002.
50. Adams P D, Afonine P V, Bunkoczi G, Chen V B, Davis I W, Echols N, et al. PHENIX: a comprehensive Python-based system for macromolecular structure solution. Acta Crystallographica Section D-Biological Crystallography. 2010; 66:213-21. doi: 10.1107/s0907444909052925. PubMed PMID: ISI: 000273820800013.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. An isolated anti-SARS-CoV-2 binder protein comprising one or more CDRs selected from the group consisting of:
(a) a CDR1 comprising an amino acid sequence having the amino acid sequence of TASGIALHX₁X₂ (SEQ ID NO:25), wherein X₁ is T, S, or H, and X₂ is H, K, or L;
(b) a CDR2 comprising an amino acid sequence having the amino acid sequence of ISSGDGTT (SEQ ID NO:3); and
(c) a CDR3 comprising an amino acid sequence having the amino acid sequence of DPX₃X₄VCHSGSYYYTDDDFYY (SEQ ID NO:26), wherein X₃ is G, S, or absent, and X₄ is A, G, or absent.

2. The isolated anti-SARS-CoV-2 binder protein of claim 1, comprising:
(a) a CDR1 comprising an amino acid sequence having the amino acid sequence of TASGIALHX₁X₂ (SEQ ID NO:25), wherein X₁ is T, S, or H, and X₂ is H, K, or L;
(b) a CDR2 comprising an amino acid sequence having the amino acid sequence of ISSGDGTT (SEQ ID NO:3); and
(c) a CDR3 comprising an amino acid sequence having the amino acid sequence of DPX₃X₄VCHSGSYYYTDDDFYY (SEQ ID NO:26), wherein X₃ is G, S, or absent, and X₄ is A, G, or absent.

3. The isolated anti-SARS-CoV-2 binder protein of claim 1, wherein X₁ is T, and X₂ is H.

4. The isolated anti-SARS-CoV-2 binder protein of claim 1, wherein X₃ is G, and X₄ is A.

5. The isolated anti-SARS-CoV-2 binder protein of claim 1, wherein X₁ is S, and X₂ is K.

6. The isolated anti-SARS-CoV-2 binder protein of claim 1, wherein X₃ is S, and X₄ is G.

7. An isolated anti-SARS-CoV-2 binder protein comprising:
(1) one or more CDRs selected from the group consisting of:
(a) a CDR1 comprising an amino acid sequence having at least 80% sequence identity to the amino acid sequence of TASGIALHTH (SEQ ID NO:2);
(b) a CDR2 comprising an amino acid sequence having at least 80% sequence identity to the amino acid sequence of ISSGDGTT (SEQ ID NO:3); and
(c) a CDR3 comprising an amino acid sequence having at least 80% sequence identity to the amino acid sequence of DPGAVCHSGSYYYTDDDFYY (SEQ ID NO:4); and/or
(2) one or more CDRs selected from the group consisting of:
(a) a CDR1 comprising an amino acid sequence having at least 80% sequence identity to the amino acid sequence of TASGIALHSK (SEQ ID NO:13) or TASGIALHHL (SEQ ID NO:18);
(b) a CDR2 comprising an amino acid sequence having at least 80% sequence identity to the amino acid sequence of ISSGDGTT (SEQ ID NO:3); and
(c) a CDR3 comprising an amino acid sequence having at least 80% sequence identity to the amino acid sequence of DPSGVCHSGSYYYTDDDFYY (SEQ ID NO:14) or DPVCHSGSYYYTDDDFYY (SEQ ID NO:19).

8. The isolated anti-SARS-CoV-2 binder protein of claim 1, comprising:
(a) a CDR1 comprising the amino acid sequence of TASGIALHTH (SEQ ID NO:2);
(b) a CDR2 comprising the amino acid sequence of ISSGDGTT (SEQ ID NO:3); and
(c) a CDR3 comprising the amino acid sequence of DPGAVCHSGSYYYTDDDFYY (SEQ ID NO: 4).

9. The isolated anti-SARS-CoV-2 binder protein of claim 1, comprising:
(a) a CDR1 comprising the amino acid sequence of TASGIALHSK (SEQ ID NO:13);
(b) a CDR2 comprising the amino acid sequence of ISSGDGTT (SEQ ID NO:3); and
(c) a CDR3 comprising the amino acid sequence of DPSGVCHSGSYYYTDDDFYY (SEQ ID NO: 14).

10. The isolated anti-SARS-CoV-2 binder protein of claim 1, comprising:
(a) a CDR1 comprising the amino acid sequence of TASGIALHHL (SEQ ID NO:18);
(b) a CDR2 comprising the amino acid sequence of ISSGDGTT (SEQ ID NO:3); and
(c) a CDR3 comprising the amino acid sequence of DPVCHSGSYYYTDDDFYY (SEQ ID NO: 19).

11. The isolated anti-SARS-CoV-2 binder protein of claim 1, wherein the binder protein comprises an anti-SARS-CoV-2 single-domain antibody (sdAb) comprising an amino acid sequence that has at least 80% sequence identity to
(SEQ ID NO: 1)
QVQLQESGGGLVQPGGSLRLSCTASGIALHTHATGWFRQAPGKEREGVS
CISSGDGTTYYEDSVEGRFTISRDNAKNTVYLQMNSLKLEDTAVYYCAA
DPGAVCHSGSYYYTDDDFYYRGQGTQVTVSS,
(SEQ ID NO: 12)
QVQLQESGGGLVQPGGSLRLSCTASGIALHSKATGWFRQAPGKEREGVS
CISSGDGTTYYEDSVEGRFTISRDNAKNTVYLQMNSLKLEDTAVYYCAA
DPSGVCHSGSYYYTDDDFYYRGQGTQVTVSS,
or
(SEQ ID NO: 17)
QVQLQESGGGLVQPGGSLRLSCTASGIALHHLATGWFRQAPGKEREGVS
CISSGDGTTYYEDSVEGRFTISRDNAKNTVYLQMNSLKLEDTAVYYCAA
DPVCHSGSYYYTDDDFYYRGQGTQVTVSS.

12. The isolated anti-SARS-CoV-2 binder protein of claim 1, wherein the binder protein comprises an anti-SARS-CoV-2 single-domain antibody (sdAb) that is linked to a Fc tag through a peptide bond or a polypeptide linker.

13. The isolated anti-SARS-CoV-2 binder protein of claim 12, comprising an amino acid sequence that has at least 90% sequence identity to any one of SEQ ID NO:6, 16, 27, 28, or 29.

14. The isolated anti-SARS-CoV-2 binder protein of claim 1, wherein the binder protein comprises a first sdAb domain and a second sdAb domain.

15. The isolated anti-SARS-CoV-2 binder protein of claim 14, which is a bispecific binder protein comprising:
the first sdAb domain that comprises
(a) a CDR1 comprising an amino acid sequence of SEQ ID NO:2;
(b) a CDR2 comprising an amino acid sequence of SEQ ID NO:3; and
(c) a CDR3 comprising an amino acid sequence of SEQ ID NO:4; and
the second sdAb domain that comprises
(a) a CDR1 comprising an amino acid sequence of SEQ ID NO:13;
(b) a CDR2 comprising an amino acid sequence of SEQ ID NO:3; and
(c) a CDR3 comprising an amino acid sequence of SEQ ID NO:14.

16. The isolated anti-SARS-CoV-2 binder protein of claim 15, wherein
the first sdAb domain comprises an amino acid sequence that has at least 80% sequence identity to SEQ ID NO:1; and
the second sdAb domain comprises an amino acid sequence that has at least 80% sequence identity to SEQ ID NO:12.

17. The isolated anti-SARS-CoV-2 binder protein of claim 14, comprising a first sdAb-Fc fusion protein that comprises a Fc tag having T366Y and a second sdAb-Fc fusion protein that comprises a Fc tag having Y407T.

18. The isolated anti-SARS-CoV-2 binder protein of claim 17, wherein the first sdAb-Fc fusion protein comprises an amino acid sequence having at least 80% sequence identity to SEQ ID NO: 27 and the second sdAb-Fc fusion protein comprises an amino acid sequence having at least 80% sequence identity to SEQ ID NO:28.

19. A pharmaceutical composition comprising the isolated anti-SARS-CoV-2 binder protein according to claim 1, and a pharmaceutically acceptable carrier.

20. An isolated polynucleotide comprising a nucleotide sequence encoding an isolated anti-SARS-CoV-2 binder protein of claim 1.

21. A vector comprising the polynucleotide of claim 20.

22. A cell comprising the polynucleotide of claim 20.

23. A method for treating or preventing a SARS-CoV-2 infection in a mammal, comprising administering an effective amount of an isolated anti-SARS-CoV-2 binder protein of claim 1 to the mammal.


SEQUENCE LISTING (43 sequences)

SEQ ID NO: 1 - protein, 129 aa, synthetic construct
QVQLQESGGGLVQPGGSLRLSCTASGIALHTHATGWFRQAPGKEREGVSCISSGDGTTYYEDSVEGRFTISRDNAKNTVYLQMNSLKLEDTAVYYCAADPGAVCHSGSYYYTDDDFYYRGQGTQVTVSS

SEQ ID NO: 2 - protein, 10 aa, synthetic construct
TASGIALHTH

SEQ ID NO: 3 - protein, 8 aa, synthetic construct
ISSGDGTT

SEQ ID NO: 4 - protein, 20 aa, synthetic construct
DPGAVCHSGSYYYTDDDFYY

SEQ ID NO: 5 - protein, 150 aa, synthetic construct
QVQLQESGGGLVQPGGSLRLSCTASGIALHTHATGWFRQAPGKEREGVSCISSGDGTTYYEDSVEGRFTISRDNAKNTVYLQMNSLKLEDTAVYYCAADPGAVCHSGSYYYTDDDFYYRGQGTQVTVSSGGQHHHHHHGAYPYDVPDYAS

SEQ ID NO: 6 - protein, 361 aa, synthetic construct
QVQLQESGGGLVQPGGSLRLSCTASGIALHTHATGWFRQAPGKEREGVSCISSGDGTTYYEDSVEGRFTISRDNAKNTVYLQMNSLKLEDTAVYYCAADPGAVCHSGSYYYTDDDFYYRGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - skipped

SEQ ID NO: 8 - protein, 6 aa, synthetic construct
HHHHHH

SEQ ID NO: 9 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 387 bp, synthetic construct
  source 1..387: mol_type = other DNA
caggtgcagctgcaggagtctgggggaggcttggtgcagcctgggggctctctgagactctcctgtacagcctctggaatcgctttgcatactcatgccacaggctggttccgccaggccccagggaaggagcgcgagggggtctcatgtattagtagtggtgatggtaccacatactatgaagactccgtggaggggcgattcaccatctccagagacaatgccaagaacacggtgtatctgcaaatgaacagcctgaaacttgaggatacggccgtttattactgtgcggcggatccgggggcggtatgccatagtggcagttactactatacggatgacgacttctactaccggggccaggggacccaggtcaccgtctcctca

SEQ ID NO: 11 - DNA, 1083 bp, synthetic construct
  source 1..1083: mol_type = other DNA
caggtgcagctgcaggagtctgggggaggcttggtgcagcctgggggctctctgagactctcctgtacagcctctggaatcgctttgcatactcatgccacaggctggttccgccaggccccagggaaggagcgcgagggggtctcatgtattagtagtggtgatggtaccacatactatgaagactccgtggaggggcgattcaccatctccagagacaatgccaagaacacggtgtatctgcaaatgaacagcctgaaacttgaggatacggccgtttattactgtgcggcggatccgggggcggtatgccatagtggcagttactactatacggatgacgacttctactaccggggccaggggacccaggtcaccgtctcctcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 12 - protein, 129 aa, synthetic construct
QVQLQESGGGLVQPGGSLRLSCTASGIALHSKATGWFRQAPGKEREGVSCISSGDGTTYYEDSVEGRFTISRDNAKNTVYLQMNSLKLEDTAVYYCAADPSGVCHSGSYYYTDDDFYYRGQGTQVTVSS

SEQ ID NO: 13 - protein, 10 aa, synthetic construct
TASGIALHSK

SEQ ID NO: 14 - protein, 20 aa, synthetic construct
DPSGVCHSGSYYYTDDDFYY

SEQ ID NO: 15 - protein, 150 aa, synthetic construct
QVQLQESGGGLVQPGGSLRLSCTASGIALHSKATGWFRQAPGKEREGVSCISSGDGTTYYEDSVEGRFTISRDNAKNTVYLQMNSLKLEDTAVYYCAADPSGVCHSGSYYYTDDDFYYRGQGTQVTVSSGGQHHHHHHGAYPYDVPDYAS

SEQ ID NO: 16 - protein, 361 aa, synthetic construct
QVQLQESGGGLVQPGGSLRLSCTASGIALHSKATGWFRQAPGKEREGVSCISSGDGTTYYEDSVEGRFTISRDNAKNTVYLQMNSLKLEDTAVYYCAADPSGVCHSGSYYYTDDDFYYRGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 127 aa, synthetic construct
QVQLQESGGGLVQPGGSLRLSCTASGIALHHLATGWFRQAPGKEREGVSCISSGDGTTYYEDSVEGRFTISRDNAKNTVYLQMNSLKLEDTAVYYCAADPVCHSGSYYYTDDDFYYRGQGTQVTVSS

SEQ ID NO: 18 - protein, 10 aa, synthetic construct
TASGIALHHL

SEQ ID NO: 19 - protein, 18 aa, synthetic construct
DPVCHSGSYYYTDDDFYY

SEQ ID NO: 20 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 22 - DNA, 387 bp, synthetic construct
  source 1..387: mol_type = other DNA
caggtgcagctgcaggagtctgggggaggcttggtgcagcctgggggctctctgagactctcctgtacagcctctggaatcgctttgcatagtaaggccacaggctggttccgccaggccccagggaaggagcgcgagggggtctcatgtattagtagtggtgatggtaccacatactatgaagactccgtggaggggcgattcaccatctccagagacaatgccaagaacacggtgtatctgcaaatgaacagcctgaaacttgaggatacggccgtttattactgtgcggcggatccaagtggggtatgccatagtggcagttactactatacggatgacgacttctactaccgggggcaggggacccaggtcaccgtctcctca

SEQ ID NO: 23 - DNA, 1083 bp, synthetic construct
  source 1..1083: mol_type = other DNA
caggtgcagctgcaggagtctgggggaggcttggtgcagcctgggggctctctgagactctcctgtacagcctctggaatcgctttgcatagtaaggccacaggctggttccgccaggccccagggaaggagcgcgagggggtctcatgtattagtagtggtgatggtaccacatactatgaagactccgtggaggggcgattcaccatctccagagacaatgccaagaacacggtgtatctgcaaatgaacagcctgaaacttgaggatacggccgtttattactgtgcggcggatccaagtggggtatgccatagtggcagttactactatacggatgacgacttctactaccgggggcaggggacccaggtcaccgtctcctcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 24 - DNA, 381 bp, synthetic construct
  source 1..381: mol_type = other DNA
caggtgcagctgcaggagtctgggggaggcttggtgcagcctgggggctctctgagactctcctgtacagcctctggaatcgctttgcatcatttggccacaggctggttccgccaggccccagggaaggagcgcgagggggtctcatgtattagtagtggtgatggtaccacatactatgaagactccgtggaggggcgattcaccatctccagagacaatgccaagaacacggtgtatctgcaaatgaacagcctgaaacttgaggatacggccgtttattactgtgcggcggatccggtatgccatagtggcagttactactatacggatgacgacttctactaccggggccaggggacccaggtcaccgtctcctca

SEQ ID NO: 25 - protein, 10 aa, synthetic construct
  VARIANT 9: note = T, S, or H
  VARIANT 10: note = H, K, or L
TASGIALHXX

SEQ ID NO: 26 - protein, 20 aa, synthetic construct
  VARIANT 3: note = G, S, or absent
  VARIANT 4: note = A, G, or absent
DPXXVCHSGSYYYTDDDFYY

SEQ ID NO: 27 - protein, 361 aa, synthetic construct
QVQLQESGGGLVQPGGSLRLSCTASGIALHTHATGWFRQAPGKEREGVSCISSGDGTTYYEDSVEGRFTISRDNAKNTVYLQMNSLKLEDTAVYYCAADPGAVCHSGSYYYTDDDFYYRGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 361 aa, synthetic construct
QVQLQESGGGLVQPGGSLRLSCTASGIALHSKATGWFRQAPGKEREGVSCISSGDGTTYYEDSVEGRFTISRDNAKNTVYLQMNSLKLEDTAVYYCAADPSGVCHSGSYYYTDDDFYYRGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 29 - protein, 359 aa, synthetic construct
QVQLQESGGGLVQPGGSLRLSCTASGIALHHLATGWFRQAPGKEREGVSCISSGDGTTYYEDSVEGRFTISRDNAKNTVYLQMNSLKLEDTAVYYCAADPVCHSGSYYYTDDDFYYRGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 6 aa, synthetic construct
GGSGGS

SEQ ID NO: 31 - protein, 4 aa, synthetic construct
RRAR

SEQ ID NO: 32 - protein, 4 aa, synthetic construct
AGAR

SEQ ID NO: 33 - protein, 62 aa, synthetic construct
ATRFASVYAWKVSGNYNYLYRLFRKSNLKPQAGNKPCNGVAGFNCYFPLRSYSFRPTYGVGH

SEQ ID NO: 34 - protein, 62 aa, synthetic construct
ATRFASVYAWKVGGNYNYRYRLFRKSNLKPQAGNKPCNGVAGVNCYFPLQSYGFRPTYGVGH

SEQ ID NO: 35 - protein, 62 aa, synthetic construct
ATTFASVYAWKPSGNYNYLYRLFRKSKLKPQAGNKPCNGVAGPNCYSPLQSYGFRPTYGVGH

SEQ ID NO: 36 - protein, 61 aa, synthetic construct
ATRFASVYAWKHSGNYDYWYRSFRKSKLKPQAGNKPCKGKGPNCYFPLQSYGFRPTYGVGH

SEQ ID NO: 37 - protein, 61 aa, synthetic construct
ATTFASVYAWKHSGNYDYWYRSLRKSKLKPQAGNKPCKGKGPNCYFPLQSYGFRPTYGVGH

SEQ ID NO: 38 - protein, 61 aa, synthetic construct
ATRFASVYAWKHSGNYDYWYRSLRKSKLKPQAGNKPCKGKGPNCYFPLESYGFRPTYGVGH

SEQ ID NO: 39 - protein, 62 aa, synthetic construct
ATRFASVYAWKVGGNYNYLYRLFRKSNLKPQAGNKPCNGVAGFNCYFPLRSYGFRPTYGVGH

SEQ ID NO: 40 - protein, 62 aa, synthetic construct
ATRFASVYAWTVGGNYNYRYRLFRKSKLKPQAGNKPCNGVAGVNCYFPLQSYGFRPTYGVGH

SEQ ID NO: 41 - protein, 62 aa, synthetic construct; identical to SEQ ID NO: 35

SEQ ID NO: 42 - protein, 62 aa, synthetic construct
ATTFASVYAWKPSGNYNYLYRLLRKSKLKPQAGNKPCNGVAGPNCYSPLQSYGFRPTYGVGH

SEQ ID NO: 43 - protein, 61 aa, synthetic construct
ATTFASVYAWRRSGNYDYWYRSLRKSKLKPQAGNKPCKGKGPDCYFPLESYGFRPTYGVGH